(19) United States
(12) Patent Application Publication
(10) Pub. No.: US 2020/0306399 A1
(43) Pub. Date: Oct. 1, 2020

(54) SYSTEMS FOR SEQUENTIAL DELIVERY OF AQUEOUS COMPOSITIONS

(71) Applicant: Markesbery Blue Pearl LLC, Hebron, KY (US)

(72) Inventors: W. Russell Markesbery, Hebron, KY (US); Eugene J. Pancheri, Cincinnati, OH (US); Larry Dean Moore, Loveland, OH (US); Daniel H. Lajiness, Fairfield, OH (US); Daniel F. Nesbitt, Cincinnati, OH (US)

(21) Appl. No.: 16/755,361

(22) PCT Filed: Oct. 11, 2018
(86) PCT No.: PCT/US2018/055367

§ 371 Date: Apr. 10, 2020

Related U.S. Application Data
(60) Provisional application No. 62/570,808, filed on Oct. 11, 2017.
(60) Provisional application No. 62/591,591, filed on Nov. 28, 2017.
(60) Provisional application No. 62/591,588, filed on Nov. 28, 2017.

(51) Int. Cl. A61L 2/18; A61L 2/10; A61L 2/22; A61L 2/24; A61L 2/03

(52) U.S. Cl. CPC A61L 2/186; A61L 2/10; A61L 2/03; A61L 2/24; A61L 2/22

(57) ABSTRACT

A method for disinfecting surfaces within a volumetric space using a peracid. The peracid is formed in a reaction layer in situ on the surface by sequentially dispersing a first composition comprising a peroxide compound and a first composition comprising an organic acid compound onto the surface, thereby preventing the peracid from being formed until the peroxide and organic acid contact each other on the surface. Delivery systems are provided for sequentially applying liquid compositions in a time-dependent manner, including associated software and hardware. An Internet of Things and single board computer assemblies can be utilized to control the sequential application of two or more liquid compositions in a time-dependent manner.

### FIELD OF THE INVENTION

The present invention is in the field of systems used in the delivery of aqueous compositions, particularly those involved in the disinfection and sterilization of surfaces.

### BACKGROUND OF THE INVENTION

There is a need for inexpensive, effective, yet safe and convenient methods to minimize the microbial burden of objects we interact with, and systems with which to apply such methods. Recently, that burden has become more severe, as several microbes have become resistant to virtually all known antibiotics. It has been predicted that we may soon enter a post-antibiotic era that will be similar to the pre-antibiotic era in which even minor infections will be life threatening. Consequently, there has been a push to disinfect and sanitize surfaces that are contaminated with bacteria that are capable of communicating diseases to humans, pets, and other beneficial life that may be exposed to them, utilizing ingredients and systems other than traditional antibiotics that are relatively safe to humans yet are still biocidal.

For centuries prior to the antibiotic era, humans had safely utilized natural biocides, including, but not limited to: vinegar, ethanol, spices, essential oils, and honey. More recently, hydrogen peroxide has been shown to fight microbes, and has long been an internal method that evolved in the animals' eternal fight against the microbes that infest them. Electricity and ultraviolet energy have also been shown to have biocidal properties. However, each biocide individually is not effective against all types of microbes, and several target microbes have developed defense mechanisms against one or more of them.

Combinations of two or more of these biocides have proven to work synergistically to enhance each one's effects. Particularly, combining hydrogen peroxide and acetic acid (the primary component of vinegar) to form peroxyacetic acid has proven to be especially effective. Several methods, apparatuses, and disinfecting systems utilizing peracids, including peroxyacetic acid, have been described in U.S. Pat. Nos. 6,692,694, 7,351,684, 7,473,675, 7,534,756, 8,110,538, 8,696,986, 8,716,339, 8,987,331, 9,044,403, 9,050,384, 9,192,909, 9,241,483, and U.S. Patent Publications 2015/0297770 and 2014/0178249, the disclosures of which are incorporated by reference in their entireties.

However, one of the biggest drawbacks with using peracids is that they are easily hydrolyzed to produce ordinary acids and either oxygen or water. Consequently, peroxyacetic acid has limited storage stability and a short shelf life. Peroxyacetic acid instability is described in detail in U.S. Pat. No. 8,034,759, the disclosure of which is incorporated by reference in its entirety. Often, commercially available products contain additional components to combat this problem, by including either a large excess of hydrogen peroxide to drive equilibrium toward the peracid form, or stabilizers such as other acids, oxidizing agents, and surfactants. Some methods have prevented degradation during shipping and storage by requiring that individual components of a peracid composition be mixed together, and subsequently applied, at the location and time that a target will be disinfected or sterilized. Yet these methods nonetheless require expensive additives that are difficult to obtain, such as polyhydric alcohols, esters, and transition metals, as well as specific reaction conditions.

As a non-limiting example of the measures taken to stabilize peracid compositions, U.S. Pat. No. 8,716,339 describes a disinfectant system that includes a first chamber containing a first solution that includes an alcohol, an organic carboxylic acid, and a transition metal or metal alloy, and a second chamber containing a second solution that includes hydrogen peroxide. Prior to disinfecting, the system is configured to mix the first and second solutions prior to dispensing the mixture onto a surface. Mixing the first and second solutions forms a peracid within the disinfectant system prior to dispensing, but the presence of the transition metal is required to help stabilize the peracid in the period between when the solutions are mixed and when the mixture reaches the contaminated surface.

The system described in U.S. Pat. No. 8,716,339, as well as countless other systems that employ peracid chemistry, form the peracid prior to dispensing it onto a surface to be disinfected. Because the issues with peracid stability have not been solved, one or more chemical components are often added to stabilize peracid compositions prior to being dispensed. These are often expensive, relatively scarce, and can have undesirable effects within the environment to be disinfected, such as the leaving of residues, films, stains, and pungent odors on treated surfaces and the environments that contain them. Even if those undesirable effects can be later remedied, there are known safety concerns associated with dispersing airborne particles or peracids into the environment in an effort to sterilize that environment. Safety data and recommended exposure levels are described in detail in Acute Exposure Guideline Levels for Selected Airborne Chemicals, National Research Council (US) Committee on Acute Exposure Guideline Levels, pg. 327-367, Volume 8, 2010, the disclosure of which is hereby incorporated by reference in its entirety.

Some automated aqueous delivery systems have been developed for dispensing potentially toxic or hazardous materials into a volumetric space, such as a room, workspace, or passenger compartment, while enabling cleaning personnel to safely monitor the progress elsewhere. However, these systems are typically either hardware-based machines having little versatility or adaptability, or are highly dedicated machines with a commensurately higher cost. As such, these machines are expensive, inefficient, and extremely difficult to adapt and utilize for those wishing to apply chemicals within spaces that can typically be accessed or inhabited by humans and/or animals.

As a result, there is still a need to develop sterilization and disinfecting methods utilizing peracids that are simultaneously effective, convenient, and safe, while at the same time using cheap and readily available materials.

### SUMMARY OF THE INVENTION

The present invention provides methods for disinfecting surfaces using peracid chemistry while eliminating instability issues and human safety issues associated with forming the peracid at any point prior to contacting a surface, by dispersing peracid reactant compounds in separate application steps and forming in situ the peracid directly on the surface.

In some embodiments, a broad and complete microbe kill is achieved through careful selection of substantially different mechanisms acting in concert with each other, in order that no microbe can develop mutations that would render future generations resistant. In further embodiments, the methods described herein can provide a prophylactic coating that can protect certain surfaces from corrosion and/or microbial contamination.

In some embodiments, a method of disinfecting a surface in need of disinfecting within a volumetric area or space is provided, comprising the steps of: a) dispensing onto the surface a first aqueous composition comprising a first peracid reactant compound that is either a peroxide compound or an organic acid compound capable of reacting with a peroxide compound to form a peracid; b) allowing a time sufficient for the first aqueous composition to distribute across the surface and coalesce into a first aqueous composition layer upon the surface; c) dispensing onto the surface a second aqueous composition comprising a second peracid reactant compound that is the other of the first peracid reactant compound; and d) allowing a second time sufficient for the second aqueous composition to combine with the coalesced first aqueous composition layer and to form a reaction layer upon the surface, thereby forming a peracid in situ within the reaction layer and disinfecting the surface.

In some embodiments, the volumetric space is a space in which humans and/or animals can access and conduct common everyday activities. Examples of such volumetric spaces include, but are not limited to: living spaces such as family rooms, bedrooms, kitchens, restrooms, basements, garages, and other rooms commonly found in one's home; classrooms; offices; retail spaces; hotel rooms; hospital rooms, operating rooms; food-operations spaces including dining, food preparation, packaging, and processing facilities; shipping containers; animal pens, factories and other industrial areas; and passenger compartments utilized in transportation, including personal vehicles, cabs, buses, subway and other rail cars, ferries, and airplanes.

In other embodiments, the volumetric space is inaccessible to humans and/or animals. Methods to disinfect surfaces within such inaccessible volumetric spaces include both clean-in-place (CIP) and clean-out-of-place (COP) options. Surfaces within inaccessible volumetric spaces that can be disinfected using CIP methods include, but are not limited to: heating, ventilation, and air conditioning (HVAC) systems; plumbing systems; and other compartments and spaces in which a human or animal cannot or generally will not enter. In another embodiment, COP methods can be utilized to disinfect the surfaces of parts that have been disassembled from the equipment they are typically housed in. In such methods, the parts can be placed on top of a surface situated in any of the volumetric spaces listed above, or inside a sealable tank, compartment, or housing, which once sealed, comprises the volumetric space.

In some embodiments, the methods of the present invention can be utilized to disinfect both porous and non-porous surfaces commonly found in the volumetric spaces listed above, including building walls, floors, ceilings, furniture, instruments, and electronics that are found within the volumetric space. In further embodiments, the surface in need of disinfecting is selected from the group consisting of plastics; metals; linoleum; tiles; vinyl; stone; structural lumber and/or finished wood; concrete; wallboards; plaster; carpet; insulation; pulp and fiber-based materials; glass; heating, ventilation, and air conditioning (HVAC) systems; plumbing; and vinyl, including combinations thereof.

In some embodiments, surfaces to disinfected can include surfaces that have been water-damaged, including not limited to water damage resulting from clogged-up or damaged plumbing, or a natural disaster such as a hurricane, tsunami, or flood. In some further embodiments, disinfecting water-damaged surface enables the surfaces to ultimately be recycled and/or reused. In other further embodiments, disinfecting water-damaged surfaces enables the surfaces to be safely collected and removed from the affected area.

In some embodiments, the first aqueous composition and the second aqueous composition are comprised of food-grade components. In further embodiments, one or more aqueous compositions are substantially free of surfactants, polymers, chelators, and metal colloids or nanoparticles.

In some embodiments, aqueous compositions of the present invention can be dispensed into the volumetric space and onto surfaces using methods commonly known to those skilled in the art, including but not limited to direct application using a mop, cloth, or sponge; streaming as a liquid stream from a hose or mechanical coarse spray device; or dispersing into the volumetric space as a multiplicity of microdroplets, including methods in which the multiplicity of microdroplets is formed when the aqueous compositions are dispersed as a vapor that has cooled and condensed into microdroplets.

In some embodiments, substantially all of the first aqueous composition is retained on the surface upon dispensing the second aqueous composition onto the surface.

In some embodiments, one or both of the first aqueous composition and the second aqueous composition are each dispensed as a liquid stream onto the surface. In further embodiments, the method further comprises the step of providing a mechanical coarse spray device, wherein the first aqueous composition and the second aqueous composition are each dispensed as a liquid stream onto the surface using the mechanical coarse spray device; particularly wherein the liquid stream is dispensed in the form of a mist, a shower, or a jet. In even further embodiments, aqueous compositions dispensed as a mist, shower, or jet can comprise macrodroplets of any size so long as the macrodroplets are capable of reaching the intended surface(s) using the particular mechanical course spray device. In still even further embodiments, the macrodroplets have an effective diameter at least about 100 microns, including at least about 250 microns, 500 microns, 1 millimeter, 2 millimeters, 3 millimeters, or 4 millimeters, and up to about 5 millimeters, including up to about 4 millimeters, 3 millimeters, 2 millimeters, 1 millimeter, 500 microns, or 250 microns. In yet still even further embodiments, at least about 90 percent of the multiplicity of microdroplets, including about 95 or 98 percent, up to about 99 percent, of the multiplicity of microdroplets has an effective diameter of at least about 100 microns, including at least about 250 microns, 500 microns, 1 millimeter, 2 millimeters, 3 millimeters, or 4 millimeters, and up to about 5 millimeters, including up to about 4 millimeters, 3 millimeters, 2 millimeters, 1 millimeter, 500 microns, or 250 microns.

In some embodiments in which the first aqueous composition and the second aqueous composition are each dispensed as a liquid stream, the time sufficient for the first aqueous composition to distribute across the surface is the time sufficient to fully immerse the surface with the first aqueous composition. In further embodiments, the second time sufficient for the second aqueous composition to distribute across the surface is the time sufficient to fully immerse the surface with the second aqueous composition. In other further embodiments, the second time sufficient for the second aqueous composition to distribute across the surface is the time sufficient for substantially all of the second peracid reactant compound to combine and react with substantially all of the first peracid reactant compound.

In some embodiments, methods of the present invention in which the first aqueous composition and the second aqueous composition are dispensed as a liquid stream can be utilized to disinfect selected surfaces within a volumetric space.

In other embodiments, the present invention provides methods for disinfecting surfaces by dispersing the first aqueous composition and the second aqueous composition as a multiplicity of microdroplets. In some embodiments, the method for disinfecting a surface in need of disinfecting within a volumetric space comprises the steps of: a) dispersing into the volumetric space a multiplicity of microdroplets of a first aqueous composition comprising a first peracid reactant compound that is either a peroxide compound or an organic acid compound capable of reacting with a peroxide compound to form a peracid; b) allowing a time sufficient for the multiplicity of microdroplets of the first aqueous composition to distribute throughout the volumetric space and to deposit and coalesce into a first aqueous composition layer upon the surface; c) dispersing into the volumetric space a multiplicity of microdroplets of a second aqueous composition comprising a second peracid reactant compound that is the other of the first peracid reactant compound; and d) allowing a second time sufficient for the multiplicity of microdroplets of the second aqueous composition to deposit onto the coalesced first aqueous composition layer to form a reaction layer upon the surface, thereby forming a peracid in situ within the reaction layer and disinfecting the surface.

In some embodiments, one or more of the aqueous compositions dispersed as a multiplicity of microdroplets have a volatility such that at least 90% of the reaction layer can evaporate within 30 minutes of being formed. In further embodiments, at least 95% of the reaction layer can evaporate, at standard conditions, within 30 minutes of being formed. In even further embodiments, at least 99% of the reaction layer can evaporate within 30 minutes of being formed. In still further embodiments, at least 99.5% of the reaction layer can evaporate within 30 minutes of being formed. In yet further embodiments, at least 99.7% of the reaction layer can evaporate within 30 minutes of being formed. In still yet further embodiments, at least 99.9% of the reaction layer can evaporate within 30 minutes of being formed.

In another embodiment, the individual components of one or more of the aqueous compositions can be selected to have vapor pressures that facilitate the evaporation of the reaction layer after sterilization of the surfaces within the volumetric space is complete. In further embodiments, one or both of the aqueous compositions can be formulated so at least about 99.0, 99.5, or 99.9% of the components by weight of the aqueous composition have a vapor pressure of at least 1.0 mm Hg at 20° C. In even further embodiments, one or both of the aqueous compositions can be formulated so that essentially 100% of the components by weight of the aqueous composition have a vapor pressure of at least about 1.0 mm Hg at 20° C.

In some embodiments, the effective diameter of the multiplicity of microdroplets is controlled to be small enough to allow the microdroplets to reach a diversity of the intended surfaces to be disinfected within a volumetric space, and to be large enough to minimize deep lung penetration if the microdroplets were to be inhaled. In other embodiments, a preponderance of the multiplicity of microdroplets dispersed into the volumetric space has an effective diameter of at least about 1 micron, including at least about 5, 10, 15, 20, 25, 30, 35, 40, 45, 50, 60, 70, 80, or 90 microns, and up to about 100 microns, including up to about 90, 80, 70, 60, 50, 40, 35, 30, 25 or 20 microns. In further embodiments, at least about 90 percent of the multiplicity of microdroplets, including about 95 or 98 percent, up to about 99 percent, of the multiplicity of microdroplets has an effective diameter of at least about 1 micron, including at least about 5, 10, 15, 20, 25, 30, 35, 40, 45, 50, 60, 70, 80, or 90 microns, and up to about 100 microns, including up to about 90, 80, 70, 60, 50, 40, 35, 30, 25 or 20 microns. In even further embodiments, at least about 90 percent of the multiplicity of microdroplets, including about 95 or 98 percent, up to about 99 percent, of the multiplicity of microdroplets has an effective diameter of at least about 10 microns, and up to about 25 microns. In still further embodiments, the at least about 90 percent of the multiplicity of microdroplets, including about 95 or 98 percent, up to about 99 percent, of the multiplicity of microdroplets has an effective diameter of about 15 microns.

In some embodiments, the coalesced layer of the first and second aqueous compositions have, respectively, an effective uniform thickness. In further embodiments, the coalesced layer has an effective uniform thickness of at least about 1 micron, including at least about 2, or 3, or 5, or 8, or 10, or 15, microns, and up to about 50 microns, including up to about 40, or 30 or 20 microns. In even further embodiments, the coalesced layer has an effective uniform thickness of about 3 microns to about 8 microns.

In some embodiments, the coalesced layer of the first and second aqueous compositions have, respectively, an effective uniform thickness greater than about 50 microns, such as when applying the first and second aqueous compositions with a mechanical coarse spray device.

In some embodiments, the multiplicity of microdroplets of the first aqueous composition are electrostatically charged.

In some embodiments, the multiplicity of microdroplets of the second aqueous composition are electrostatically charged. In further embodiments, the multiplicity of microdroplets of the first aqueous composition are electrostatically charged, and the multiplicity of microdroplets of the second aqueous composition are electrostatically charged with the opposite polarity of the multiplicity of microdroplets of the first aqueous composition.

In some embodiments, the electrostatic charge of the multiplicity of microdroplets of the first aqueous composition and the second aqueous composition are optimized to provide the most desirable reaction of the first and second peracid reactant compounds. In further embodiments, the multiplicity of microdroplets of the aqueous composition comprising the peroxide compound are dispersed with a negative charge. In other embodiments, the multiplicity of microdroplets of the aqueous composition comprising the organic acid compound are dispersed with a positive charge.

In some embodiments, the surface in need of disinfecting is galvanically grounded. In further embodiments, the surface in need of disinfected is earth-grounded.

In other embodiments, the multiplicity of microdroplets of the first aqueous composition and the second aqueous composition are formed by heating the first aqueous composition and the second aqueous composition to produce a vapor phase comprising the respective peracid reactant compound in the ambient air, and allowing a time sufficient for the vapor phase comprising the peracid reactant compound to distribute throughout the volumetric space, and to cool and condense into liquid microdroplets.

In some embodiments, the first aqueous composition and the second aqueous composition are heated, separately, to a temperature of greater than about 250° C. Alternatively, the first aqueous composition and the second aqueous composition are heated, separately, to a temperature sufficient to vaporize the mass of the first aqueous composition and the second aqueous composition in a vaporizing time of less than about 30 minutes, including less than about 25, less than about 20, less than about 15, less than about 10, or less than about 5, minutes. In a further embodiment, the first aqueous composition and the second aqueous composition are heated, separately, to a temperature sufficient to vaporize the mass of the first aqueous composition and the second aqueous composition in about two minutes.

In some embodiments, the first aqueous composition and the second aqueous composition in the vapor phase are, separately, cooled to a temperature of less than about 55° C. to condense into microdroplets and deposit onto surfaces within the volumetric space to be disinfected.

In some embodiments, the first aqueous composition in the vapor phase is formed by introducing the first aqueous composition into a first hot gaseous stream, and the second aqueous composition in the vapor phase is formed by introducing the second aqueous composition into a second hot gaseous stream.

In some embodiments, the methods of the present invention can be used to simultaneously disinfect all of the surfaces within a volumetric space.

In some embodiments, the stoichiometric amount of the dispersed peroxide compound is equal to or greater than the stoichiometric amount of the dispersed organic acid compound.

In some embodiments, the pH of the composition comprising the organic acid compound is less than or equal to about 7. In further embodiments, the pH of the reaction layer is less than or equal to about 7.

In some embodiments, the organic acid compound can include any organic acid capable of forming a peracid upon reacting with a peroxide compound. In further embodiments, the aqueous composition comprising the organic acid compound comprises at least about 0.5% by weight of the organic acid compound, including at least about 1, 2, 5, 10, 15, 20, 25, 30, 35, 40, or 45% by weight, and up to about 50% by weight, including up to about 1, 2, 5, 10, 15, 20, 25, 30, 35, 40, or 45% by weight. In even further embodiments, the aqueous composition comprising the organic acid compound comprises about 2% to about 20% by weight of the organic acid compound. In still further embodiments, the aqueous composition comprising the organic acid compound comprises about 10% by weight of the organic acid compound. In yet further embodiments, the organic acid compound is dispersed within the second aqueous composition.

In some embodiments, the organic acid compound has one or more carboxylic acid functional groups. In further embodiments, the organic carboxylic acid is selected from the group consisting of: formic acid, acetic acid, citric acid, succinic acid, oxalic acid, propanoic acid, lactic acid, butanoic acid, pentanoic acid, and octanoic acid. In even further embodiments, the organic acid compound is acetic acid.

In some embodiments, the peroxide compound can include such non-limiting peroxides as hydrogen peroxide, metal peroxides, and ozone. In further embodiments, the aqueous composition comprising the peroxide compound comprises at least about 0.1% by weight of the peroxide compound, including at least about 0.5, 1, 2, 4, 6, 8, 10, 12, 14, 16, 18, or 20% by weight, up to about 25% by weight, including up to about 20, or 15 or 12% by weight. In even further embodiments, the aqueous composition comprising the peroxide compound comprises at least about 5%, and up to about 15% by weight of the peroxide compound. In still further embodiments, the aqueous composition comprising the peroxide compound comprises about 10% of the peroxide compound. In yet further embodiments, the peroxide compound is hydrogen peroxide. In still yet further embodiments, hydrogen peroxide is dispersed within the first aqueous composition.

In some embodiments, at least one of the first aqueous composition or the second aqueous composition further comprises an alcohol comprising one or more alcohol compounds. In further embodiments, the aqueous composition comprises at least about 0.05% by weight alcohol, including at least about 0.1, 1, 5, 10, 15, 20, 25, 30, 40, 50, or 60%, by weight, and up to about 70% by weight, including up to about 65, or 60, or 55, or 50, or 45 or 40 or 35, or 30, or 25, or 20%, by weight. In even further embodiments, the aqueous composition comprises at least about 1%, and up to about 25%, by weight of alcohol. In still further embodiments, the aqueous composition comprises about 15% by weight of alcohol. In yet further embodiments, the alcohol comprises at least one lower-chain alcohol selected from the group consisting of ethanol, isopropanol, and t-butanol, and mixtures thereof. In yet still further embodiments, the alcohol comprises isopropanol.

In some embodiments, at least one of the first aqueous composition or the second aqueous composition further comprises one or more natural biocides. As a non-limiting example, such compounds include manuka honey and/or essential oils. In further embodiments, the essential oils are selected from the essential oils of oregano, thyme, lemongrass, lemons, oranges, anise, cloves, aniseed, cinnamon, geraniums, roses, mint, peppermint, lavender, citronella, eucalyptus, sandalwood, cedar, rosmarin, pine, vervain fleagrass, and ratanhiae, including combinations thereof. In even further embodiments, the aqueous composition comprises about 0.001% to about 1% by weight of the natural biocide.

In other embodiments, at least one of the first aqueous composition or the second aqueous composition further comprises one or more natural biocidal compounds commonly found within manuka honey and essential oils. In further embodiments, the natural biocidal compounds are selected from the group consisting of methylglyoxal, carvacrol, eugenol, linalool, thymol, p-cymene, myrcene, borneol, camphor, caryophillin, cinnamaldehyde, geraniol, nerol, citronellol, and menthol, including combinations thereof. In even further embodiments, the aqueous composition comprises about 0.001% to about 1% by weight of the natural biocidal compound.

In some embodiments, the method further includes the step of illuminating at least one of the first aqueous composition, the second aqueous composition, and the reaction layer with a wavelength consisting essentially of ultraviolet light.

Additionally, the present invention provides methods in which one or more supplemental aqueous compositions can be dispersed into a volumetric space in addition to the first aqueous composition and the second aqueous composition. In some embodiments, methods to disinfect a surface in need of disinfecting within a volumetric space comprise the steps of: a) dispersing into the volumetric space a multiplicity of microdroplets of a first aqueous composition comprising a first peracid reactant compound that is either a peroxide compound or an organic acid compound capable of reacting with a peroxide compound to form a peracid; b) allowing a time sufficient for the multiplicity of microdroplets of the first aqueous composition to distribute throughout the volumetric space and to deposit and coalesce into a first aqueous composition layer upon the surface; c) dispersing into the volumetric space a multiplicity of microdroplets of a second aqueous composition comprising a second peracid reactant compound that is the other of the first peracid reactant compound; and d) allowing a second time sufficient for the multiplicity of microdroplets of the second aqueous composition to deposit onto the coalesced first aqueous composition layer to form a reaction layer upon the surface, thereby forming a peracid in situ within the reaction layer and disinfecting the surface, wherein the method further includes the steps of dispersing into the volumetric space one or more supplemental aqueous compositions and allowing a time sufficient for each dispersed supplemental aqueous composition to distribute throughout the volumetric space and to deposit onto the surface.

In some embodiments, a supplemental aqueous composition is dispersed into the volumetric space prior to dispersing the first aqueous composition into the volumetric space, after the first aqueous composition layer has been dispersed and is at least partially or substantially completely formed upon the surface and prior to dispersing the second aqueous composition into the volumetric space, after the second aqueous composition layer has been dispersed and is at least partially or substantially completely formed upon the surface, and/or after the peracid has been formed in situ within the reaction layer on the surface, including combinations thereof. In other embodiments, the supplemental aqueous composition can be dispersed into the volumetric space in response to the entry of a person or animal into the volumetric space while disinfection is in progress.

In some embodiments, each supplemental aqueous composition is selected from the group consisting of a peracid scavenging composition, a pesticide composition, and an environmental conditioning composition.

In some embodiments, the peracid scavenging composition comprises a metal halide compound, and the peracid scavenging composition is dispersed after the peracid has been formed in situ within the reaction layer on the surface, wherein the metal halide compound comprises iodide, bromide, or chloride, particularly a metal halide compound selected from the group consisting of potassium iodide, potassium chloride, and sodium chloride, and more particularly potassium iodide. In further embodiments, the peracid scavenging composition comprises less than about 6 moles per liter potassium iodide, including less than about 1, or 0.1, or 0.01, or 0.001, or 0.0001, or about 0.00001 moles per liter potassium iodide, down to less than about 0.000001 moles per liter potassium iodide. In even further embodiments, a stoichiometric amount of the metal halide compound is dispersed that is equal to or greater than a stoichiometric amount of the peracid formed in situ within the reaction layer, thereby scavenging substantially all of the formed peracid from the surface.

In some embodiments, the pesticide composition comprises at least one fungicide, rodenticide, herbicide, larvicide, or insecticide, including combinations thereof, particularly an insecticide configured to kill bed bugs or termites. In some embodiments, the pesticide composition is dispersed into the volumetric space prior to dispersing the first aqueous composition into the volumetric space. In other embodiments, the pesticide composition is dispersed into the volumetric space after the peracid has been formed in situ within the reaction layer on the surface.

In some embodiments, the environmental conditioning composition comprises water. In further embodiments, the environmental conditioning composition consists essentially of water. In other further embodiments, the environmental conditioning composition is reactively inert with respect to either of the peracid reactant compounds and/or the formed peracid.

In some embodiments, an environmental conditioning composition consisting essentially of water is dispersed into the volumetric space prior to dispersing the first aqueous composition into the volumetric space, in order to increase the humidity in the volumetric space to stabilize or maintain the size and composition of the microdroplets of aqueous compositions containing peracid reactant compounds, and to limit or prevent the volatile components of the microdroplets from being lost or evaporated into the environment or the volumetric space before the microdroplets of the peracid reactant compounds reach or arrive, and deposit onto, the surface to be disinfected. In further embodiments, the time sufficient for the environmental conditioning composition to distribute throughout the volumetric space is the time sufficient to cause the volumetric space to have a relative humidity of at least about 50 percent, including at least about 60, 70, 80, 90, or 95 percent, up to about 99 percent.

In other embodiments, an environmental conditioning composition consisting essentially of water is dispersed into the volumetric space after the first aqueous composition layer is formed upon the surface and prior to dispersing the second aqueous composition into the volumetric space, in order to coalesce with and enhance deposition of any excess or lingering microdroplets of the first aqueous composition from the air.

In other embodiments, an environmental conditioning composition consisting essentially of water is dispersed into the volumetric space after the peracid has been formed in situ within the reaction layer on the surface, in order to coalesce with and enhance deposition of any excess or lingering microdroplets of the second aqueous composition.

In other embodiments, the environmental conditioning composition further consists essentially of a fragrant compound, and the environmental conditioning composition is dispersed into the volumetric space after the peracid has been formed in situ within the reaction layer on the surface. In further embodiments, the fragrant compound is selected from the group consisting of methylglyoxal, carvacrol, eugenol, linalool, thymol, p-cymene, myrcene, borneol, camphor, caryophillin, cinnamaldehyde, geraniol, nerol, citronellol, and menthol, including combinations thereof.

In some embodiments, an environmental conditioning composition consisting essentially of water and a fragrant compound can be dispersed into the volumetric space after the peracid has been formed in situ within the reaction layer on the surface.

In some embodiments, one or more of the supplemental aqueous compositions are dispersed into the volumetric space as a multiplicity of microdroplets. In further embodiments, multiplicity of microdroplets of the supplemental aqueous composition is electrostatically charged. In even further embodiments, the electrostatically-charged microdroplets of the supplemental aqueous composition are negatively charged.

In some embodiments, the effective diameter of a preponderance of the microdroplets of a supplemental aqueous composition is at least about 1 micron, at least about 5, 10, 15, 20, 25, 30, 35, 40, 45, 50, 60, 70, 80, or 90 microns, and up to about 100 microns, including up to about 90, 80, 70, 60, 50, 40, 35, 30, 25 or 20 microns. In further embodiments, at least about 90 percent of the multiplicity of microdroplets, including about 95 or 98 percent, up to about 99 percent, of the multiplicity of microdroplets has an effective diameter of at least about 1 micron, including at least about 5, 10, 15, 20, 25, 30, 35, 40, 45, 50, 60, 70, 80, or 90 microns, and up to about 100 microns, including up to about 90, 80, 70, 60, 50, 40, 35, 30, 25 or 20 microns. In even further embodiments, at least about 90 percent of the multiplicity of microdroplets, including about 95 or 98 percent, up to about 99 percent, of the multiplicity of microdroplets has an effective diameter of at least about 10 microns, and up to about 25 microns. In still further embodiments, the at least about 90 percent of the multiplicity of microdroplets, including about 95 or 98 percent, up to about 99 percent, of the multiplicity of microdroplets has an effective diameter of about 15 microns.

In some embodiments, the time sufficient for the first aqueous composition, the second aqueous composition, and any of the supplemental aqueous compositions to distribute throughout the volumetric space, deposit onto the surface, and/or form an aqueous composition layer or reaction layer upon the surface is a defined passage of time. In further embodiments, the time sufficient is at least about 1 second, including at least about 10 seconds, 30 seconds, 1 minute, 2 minutes, 3 minutes, 4 minutes, 5 minutes, or 10 minutes, and up to at least about 60 minutes, including up to about 30 minutes, or 15 minutes.

Additionally, the present invention provides a safer and potentially more effective method for disinfecting or sanitizing surfaces within a volumetric space in which a pre-formed peracid is dispersed. In some embodiments, a method for disinfecting a surface in need of disinfecting within a volumetric space comprises the steps of: a) dispersing into the volumetric space a multiplicity of microdroplets of a first aqueous composition comprising a peracid; and b) allowing a time sufficient for the first aqueous composition to distribute throughout the volumetric space and to deposit onto the surface, thereby disinfecting the surface; wherein the method further includes the step of dispersing into the volumetric space a multiplicity of microdroplets of one or more supplemental aqueous compositions selected from the group consisting of a peracid scavenging composition, a pesticide composition, and an environmental conditioning composition, and allowing a time sufficient for each dispersed supplemental aqueous composition to distribute throughout the volumetric space and to deposit onto the surface. In further embodiments, the peracid is peroxyacetic acid.

In some embodiments, excess peracid lingering in the volumetric space or on the surface after sterilization is complete can be neutralized or removed by dispersing into the volumetric space a peracid scavenging composition comprising a metal halide compound after the first aqueous composition has deposited onto the surface, and allowing a time sufficient for the peracid scavenging composition to distribute throughout the volumetric space and to deposit onto the surface, wherein the metal halide compound comprises iodide or chloride, particularly a metal halide compound selected from the group consisting of potassium iodide, potassium chloride, and sodium chloride, and more particularly potassium iodide. In further embodiments, the peracid scavenging composition comprises less than about 6 moles per liter potassium iodide, including less than about 1, 0.1, 0.01, 0.001, 0.0001, or about 0.00001 moles per liter potassium iodide, down to less than about 0.000001 moles per liter potassium iodide. In even further embodiments, a stoichiometric amount of the metal halide compound is dispersed into the volumetric space that is equal to or greater than a stoichiometric amount of the peracid dispersed into the volumetric space, thereby scavenging substantially all of the peracid from the volumetric space.

In some embodiments, an environmental conditioning composition consisting essentially of water is dispersed into the volumetric space prior to dispersing the first aqueous composition comprising a peracid into the volumetric space, in order to increase the humidity in the volumetric space to stabilize or maintain the size and composition of the microdroplets of aqueous compositions containing the peracid, and to limit or prevent the volatile components of the microdroplets from being lost or evaporated into the environment or the volumetric space before the microdroplets of the first aqueous composition comprising the peracid reaches the surface. In other embodiments, an environmental conditioning composition consisting essentially of water is dispersed into the volumetric space after the first aqueous composition comprising a peracid has deposited onto the surface. In a further embodiment, the environmental conditioning composition further consists essentially of a fragrant compound. In an even further embodiment, the fragrant compound is selected from the group consisting of methylglyoxal, carvacrol, eugenol, linalool, thymol, p-cymene, myrcene, borneol, camphor, caryophillin, cinnamaldehyde, geraniol, nerol, citronellol, and menthol, including combinations thereof.

In some embodiments, the multiplicity of microdroplets of the first aqueous composition comprising a peracid is electrostatically charged. In further embodiments, the electrostatically-charged microdroplets of the first aqueous composition are negatively charged.

In some embodiments, the multiplicity of microdroplets of at least one of the first aqueous composition or the one or more supplemental aqueous compositions is formed by first heating the aqueous composition to produce a vapor and allowing a time sufficient for the vapor to distribute throughout the volumetric space and to cool and condense into microdroplets.

In some embodiments, the method to disinfect the surface further includes the step of illuminating at least one of the first aqueous composition and the surface with a wavelength consisting essentially of ultraviolet light.

The present invention also provides sequential application and delivery systems for sequentially dispensing a plurality of liquid compositions into a volumetric space in a time-dependent manner. In some embodiments, the sequential application and delivery system comprises a plurality of aqueous composition containers, each configured for housing or containing an aqueous composition; a plurality of pumps, each pump in fluid communication respectively with one of the aqueous composition containers therewith; and one or more aqueous composition delivery nozzles, each aqueous composition delivery nozzle in fluid communication with at least one pump and configured to sequentially dispense one or more aqueous compositions into a volumetric space.

In some embodiments, the liquid compositions are aqueous compositions. In other embodiments, the liquid compositions are non-aqueous compositions, including but not limited to oil-based compositions, organic compounds or compositions, and other volatile compounds or compositions that are substantially free of water.

In some embodiments, the sequential application and delivery system comprises a first aqueous composition container for housing and containing a first aqueous composition and a second aqueous composition container for housing and containing the second aqueous composition. In further embodiments, the first aqueous composition comprises a peracid reactant compound selected from the group consisting of a peroxide compound and an organic acid compound that is capable of reacting with the peroxide compound to form a peracid, and the second aqueous composition comprises the peracid reactant compound that is the other of the first peracid reactant compound.

In some embodiments, the sequential application and delivery system is configured to prevent the first aqueous composition and the second aqueous composition from contacting each other until after each aqueous composition is dispensed into the volumetric space. In further embodiments, the sequential application and delivery system is configured to prevent the first aqueous composition and the second aqueous composition from contacting each other until after each aqueous composition has deposited and/or coalesced into a layer upon the surface.

In some embodiments, the sequential application and delivery system further comprises a data acquisition and control system, including: a means for detecting the volume of the aqueous composition within each of the aqueous composition containers; a data acquisition bus; a control bus; and a controller electrically coupled to the aqueous composition containers and configured to read the means for detecting the volume of the aqueous composition within each of the aqueous composition containers. In further embodiments, the means for detecting the volume of the aqueous composition include float, capacitance, conductivity, ultrasonic, radar level, and optical sensors. In even further embodiments, each pump within the sequential application and delivery system includes a drive electrically coupled to the controller through the control bus, wherein the drive is configured to engage the pumps to dispense aqueous compositions from the aqueous composition containers to and through the aqueous composition delivery nozzles into the volumetric space.

In some embodiments, the sequential application and delivery system further comprises one or more sensors proximate or adjacent to the volumetric space and in data communication with the data acquisition bus, wherein the at least one sensor comprises a means for detecting at least one environmental condition within the volumetric space, selected from the group consisting of motion detectors, global positioning system (GPS) detectors, infrared sensors, audio sensors, thermal sensors, hygrometers, accelerometers, cameras, or light sensors, particularly laser light sensors, including combinations thereof. In further embodiments, the controller is programmed to cease dispensing an aqueous composition upon a sensor detecting the presence of an animal or human within the volumetric space. In other further embodiments, the sensor is configured to detect the Cartesian dimensions of the volumetric space and communicate the detected dimensions to the controller through the data acquisition bus.

In some embodiments, the controller is programmed to delay for a defined time after dispensing the first aqueous composition into the volumetric space before dispensing the second aqueous composition into the volumetric space. In further embodiments, the delay is at least about 1 second, including about 10 seconds, 30 seconds, 1 minute, 2 minutes, 3 minutes, 4 minutes, 5 minutes, or 10 minutes, up to at least about 30 minutes, including up to about 15 or 10 or 5 minutes.

In some embodiments, a portion of the sequential application and delivery system is coupled to a mobilized conveyance selected from the group consisting of a hand-carried dispensing unit, backpack, cart, trolley, particularly an optically-controlled or directed trolley, robot, or drone.

In some embodiments, the one or more aqueous composition delivery nozzles of the sequential application and delivery system are configured to dispense the first aqueous composition and/or the second aqueous composition as a multiplicity of microdroplets. In further embodiments, the sequential application and delivery system further comprises an ionizing device proximate or adjacent to one or more of the aqueous composition delivery nozzles, the ionizing device configured to electrostatically charge a quantity of the aqueous composition dispensed by the one or more nozzles. In even further embodiments, the multiplicity of microdroplets of the first aqueous composition and/or the multiplicity of microdroplets of the second aqueous composition are electrostatically charged by the sequential application and delivery system. In still further embodiments, the ionizing device is configured to dispense the multiplicity of microdroplets of the second aqueous composition with an electrostatic charge having the opposite polarity of the multiplicity of microdroplets of the first aqueous composition.

In some embodiments, the sequential application and delivery system is configured to optimize the electrostatic charge of the multiplicity of microdroplets of the first aqueous composition and the second aqueous composition to provide the most desirable reaction of the first and second peracid reactant compounds. In further embodiments, the sequential application and delivery system is configured to disperse the multiplicity of microdroplets of the aqueous composition comprising the peroxide compound with a negative charge. In other embodiments, the sequential application and delivery system is configured to disperse the multiplicity of microdroplets of the aqueous composition comprising the organic acid compound are dispersed with a positive charge.

In some embodiments, the sequential application and delivery system further comprises a vaporizer that is located proximate or adjacent to one or more nozzles and is electrically coupled and responsive to the controller, wherein the controller is programmed to energize the vaporizer and cause the vaporizer to emit a hot gaseous stream at the aqueous composition after being dispensed from the nozzle.

In some embodiments, the sequential application and delivery system further comprises an Internet of Things (IoT) configured to engage one or more of the plurality of pumps in a sequential, timed manner. In some further embodiments, the IoT can be configured to engage any of the plurality of pumps for at least about 1 second, including at least about 10 seconds, 30 seconds, 1 minute, 2 minutes, 3 minutes, 4 minutes, 5 minutes, or 10 minutes, and up to at least about 60 minutes, including up to about 30 minutes, or about 15 minutes. In some even further embodiments, the IoT can be configured to engage any of the plurality of pumps for a time sufficient for the aqueous composition to distribute throughout the volumetric space, deposit onto the surface, and/or form an aqueous composition layer or reaction layer upon the surface. In other further embodiments, the IoT can be configured to delay for a defined time after dispensing the first aqueous composition into the volumetric space before dispensing the second aqueous composition into the volumetric space. In further embodiments, the delay is at least about 1 second, including about 10 seconds, 30 seconds, 1 minute, 2 minutes, 3 minutes, 4 minutes, 5 minutes, or 10 minutes, up to at least about 30 minutes, including up to about 15 or 10 or 5 minutes.

In some embodiments, the IoT comprises one or more remotely-controlled outlets configured for sequentially engaging the one or more of the plurality of pumps in the sequential application and delivery system. In further embodiments, the IoT comprises at least two remotely-controlled outlets, each remotely-controlled outlet configured for sequentially energizing at least one of the plurality of pumps.

In some embodiments, the one or more remotely-controlled outlets are in direct communication with the Internet, and the IoT further comprises at least one mobile device and/or at least one computer in electronic communication with the Internet. In further embodiments, the mobile device and/or computer includes an operating system, a home automation application configured to run on the operating system, and a routine created within the home automation application that is configured to actuate the one or more remotely controlled outlets to engage the one or more of the plurality of pumps in a sequential and time-dependent manner.

In other embodiments, the one or more remotely-controlled outlets are in direct communication with an intranet and the IoT further comprises a hub in electronic communication with the intranet. In further embodiments, the hub comprises an operating system, a home automation application configured to run on the operating system, and a routine created within the home automation application and configured to actuate the one or more remotely controlled outlets to engage the one or more of the plurality of pumps in a sequential timed manner. In even further embodiments, the IoT further comprises a mobile device in electronic communication with the intranet, the mobile device comprising an operating system, a home automation application configured to run on the operating system, and a routine created within the home automation application and configured to actuate the one or more remotely controlled outlets to engage the one or more of the plurality of pumps in a sequential timed manner.

In some embodiments, the IoT further comprises one or more sensors in direct wireless electronic communication with the Internet or intranet, the one or more sensors configured to sense environmental conditions within the volumetric space, selected from the group consisting of: motion detectors; global positioning system detectors; infrared sensors; audio sensors; thermal sensors; accelerometers; light sensors, particularly laser light sensors; and cameras; including combinations thereof.

In some embodiments, the sequential application and delivery system further comprises a single board computer assembly (SBC) configured to engage one or more of the plurality of pumps in a sequential timed manner. In further embodiments, the SBC comprises a hardware attached on top (HAT) circuit board having one or more relays, each relay respectively associated with one or more of the plurality of pumps and configured to pass electric power to the respective one or more of the plurality of pumps in a sequential timed manner. In even further embodiments, the HAT circuit board has at least two relays, each relay respectively associated with one or more of the plurality of pumps and configured to pass electric power one or more of the plurality of pumps in a sequential timed manner.

In some embodiments, the SBC further comprises a display, the display having a user interface for engaging one or more of the plurality of pumps in a sequential timed manner.

In some embodiments, the SBC is in electronic communication with a mobile device configured for engaging one or more of the plurality of pumps in a sequential timed manner.

Additionally, the invention provides a kit for use in disinfecting a surface in need of disinfecting within a volumetric space, comprising: a) a first aqueous composition comprising a first peracid reactant compound that is either a peroxide compound or an organic acid compound capable of reacting with a peroxide compound to form a peracid; b) a second aqueous composition comprising a second peracid reactant compound that is the other of the first peracid reactant compound; and c) instructions comprising any of the methods described above, wherein the kit is arranged such that the first aqueous composition and the second aqueous composition are packaged separately and are not combined until the first aqueous composition and the second aqueous composition are applied sequentially onto the surface to form a reaction layer comprising the first aqueous composition and the second aqueous composition, thereby forming a peracid in situ within the reaction layer and disinfecting the surface.

In some embodiments, the kit further comprises any of the sequential application and delivery systems described above for sequentially dispensing the first aqueous composition and the second aqueous composition. In further embodiments, the sequential application and delivery system comprises an IoT.

In some embodiments, the first aqueous composition and the second aqueous composition within the kit are substantially free of surfactants, polymers, chelators, and metal colloids or nanoparticles. Any of the above-described aqueous compositions and/or components can be included with the kit, including any of the supplemental aqueous compositions, so long the included aqueous compositions are substantially free of detectable peracids, and peracids are only formed in situ on the surface(s) to be disinfected in accordance with instructions provided with the kit.

In some embodiments, the application of the first aqueous composition and the second aqueous composition achieve a log-6 or greater kill of microbes.

These and other embodiments of the present invention will be apparent to one of ordinary skill in the art from the following detailed description.

### BRIEF DESCRIPTION OF THE FIGURES

FIG. 1 shows an illustration of the commercial electrospray device according to the prior art.

FIG. 2 shows the dispersion and distribution of identically electrostatically-charged microdroplets onto a surface in need of disinfecting.

FIG. 3 shows a fluid process diagram of a sequential application and delivery system in accordance with principles of the present invention.

FIG. 4 shows a data acquisition and control signal schematic block diagram of the sequential application and delivery system shown in FIG. 3.

FIG. 5 shows a fluid process diagram of an alternative embodiment of a sequential application and delivery system in accordance with principles of the present invention.

FIG. 6 shows a data acquisition and control signal schematic block diagram of the sequential application and delivery system shown in FIG. 5.

FIG. 7 shows a fluid process diagram of another alternative embodiment of a sequential application and delivery system in accordance with principles of the present invention.

FIG. 8 shows a data acquisition and control signal schematic block diagram of the sequential application and delivery system shown in FIG. 7.

FIG. 9 shows a pictorial illustration of an Internet-based sequential application and delivery system in accordance with principles of the present invention.

FIG. 10 shows a pictorial illustration of an intranet-based sequential application and delivery system in accordance with principles of the present invention.

FIG. 11 shows a pictorial illustration of an access point based single board computer based sequential application and delivery system in accordance with principles of the present invention.

FIG. 12 shows a block diagram illustrating an exemplary software architecture for the mobile device shown in FIG. 9.

FIG. 13 shows plots illustrating the distribution of acetic acid as a function of changes in x, y, and z direction from the nozzle on an electrospray device.

FIG. 14 shows plots illustrating the independent effect of several experimental variables on the percent kill of bacteria.

FIG. 15 shows plots illustrating the correlative effect of several experimental variables on the percent kill of bacteria.

### DETAILED DESCRIPTION OF THE INVENTION

The present disclosure includes methods for sterilizing rooms, enclosed areas and volumetric spaces, and surfaces within those areas or spaces, using peracids. In some embodiments, peracids are formed in situ on those surfaces by applying peracid reactant compounds sequentially in two or more separate applications. The methods in which a peracid is formed in situ on surfaces to be disinfected have several advantages over conventional disinfecting systems requiring the application of pre-formed peracids. Limitations of present methods and systems that use a pre-formed acid to disinfect surfaces include, but are not limited to, instability of the peracid in solution, loss of the peroxyacid activity and potency, increased toxicity, and ballooning costs. To account for the instability of the peracid and its associated loss of activity, conventional disinfecting methods and systems often require adding additional peracid reactants or stabilizers to the pre-formed peracid to extend its shelf life. However, adding such stabilizers exacerbates the toxicity and cost, thus increasing the level of expertise necessary to user peracids directly. In contrast, methods of the present invention do not require stabilizers because reactant compounds used to form the peracid can be applied individually and sequentially to the surface to be disinfected. Consequently, the peracid is only formed on the target surface, disinfecting the surface with maximum potency and safety to users and bystanders alike.

The present disclosure also includes apparatuses and systems that are configured to dispense sequentially, and substantially not simultaneously, two or more aqueous compositions onto one or more surfaces within a volumetric space, whereupon reaching the surface(s) the two or more aqueous compositions interact to form a peracid in situ on the surface.

It should be understood that while reference is made to exemplary embodiments and specific language is used to describe them, no limitation of the scope of the invention is intended. Further modifications of the methods and system described herein, as well as additional applications of the principles of those inventions as described, which would occur to one skilled in the relevant art and having possession of this disclosure, are to be considered within the scope of this invention. Furthermore, unless defined otherwise, all technical and scientific terms used herein have the same meaning as commonly understood by one of ordinary skill in the art to which embodiments of this particular invention pertain. The terminology used is for the purpose of describing those embodiments only, and is not intended to be limiting unless specified as such.

### Definitions

As used in this specification and in the claims, the singular forms “a,” “an,” and “the” include plural referents unless the content clearly dictates otherwise.

The term “about” refers to variation in the numerical quantity that can occur, for example, through typical measuring and liquid handling procedures used for making concentrates or use solutions in the real world; through inadvertent error in these procedures; through differences in the manufacture, source, or purity of the ingredients used to make the compositions or carry out the methods; and the like. The term “about” also encompasses amounts that differ due to different equilibrium conditions for a composition resulting from a particular initial mixture. Similarly, whether or not a claim is modified by the term, “about,” the claims included equivalents to the quantities recited.

The term “aqueous composition” refers to a combination of liquid components that includes water. Most commonly, aqueous compositions are synonymous with the term “solution” as it is commonly used in the art for this invention. However, depending on the identity of components in the composition in addition to water, “aqueous compositions” can also encompass mixtures, emulsions, dispersions, suspensions or the like. Furthermore, while water must be present, it need not comprise the majority of the aqueous composition.

The terms “biocide” and “biocidal compound” refer to chemical substances intended to destroy, deter, render harmless, or exert a controlling effect on any organisms that are harmful to human or animal health or that cause damage to natural or manufactured products. Non-limiting examples of biocides include peroxide compounds, organic acid compounds, peracids, alcohols, manuka honey, essential oils, and natural biocidal compounds.

The term “effective diameter” refers to either the geometric diameter of a spherical droplet, or of the distance from side-to-side of a distorted spherical droplet at the droplet's widest point, and can be used to describe both microdroplets having an effective diameter of less than 100 microns, or macrodroplets having an effective diameter of greater than 100 microns.

The term “effective uniform thickness” refers to target or ideal thickness of a liquid onto a surface where the mass or volume of a liquid deposited onto the surface has a substantially uniformly thickness.

The terms “essential oil” or “spice oil” refer to concentrated natural products produced by and extracted from aromatic plants for their antimicrobial properties based on interactions with a variety of cellular targets.

The phrase “food processing surface” refers to a surface of a tool, a machine, equipment, a shipping container, railcar, structure, building, or the like that is employed as part of a food transportation, processing, preparation, or storage activity. Examples of food processing surfaces include surfaces of food processing or preparation equipment (e.g. slicing, canning, or transport equipment, including flumes), of food processing wares (e.g. utensils, dishware, wash ware, and bar glasses), and of floors, walls, or fixtures of structures in which food processing occurs. Food processing surfaces are found and employed in food anti-spoilage air circulation systems, aseptic packaging sanitizing, food refrigeration and cooler cleaners, and sanitizers, ware washing sanitizing, blancher cleaning and sanitizing, food packaging materials, cutting board additives, third-sink sanitizing, beverage chillers and warmers, meat chilling or scalding waters, autodish sanitizers, sanitizing gels, cooling towers, food processing antimicrobial garment sprays, and non-to-low-aqueous food preparation lubricants, oils, and rinse additives.

The phrase “food product” includes any food substance that might require treatment with an antimicrobial agent or composition that is edible with or without further preparation. Food products include meat (e.g. red meat and pork), seafood, poultry, produce (e.g. fruits and vegetables), eggs, living eggs, egg products, ready-to-eat food, wheat, seeds, roots, tubers, leaves, stems, corns, flowers, sprouts, seasonings, or a combination thereof. The term, “produce,” refers to food products such as fruits and vegetables and plants or plant-derived materials that are typically sold uncooked and, often, unpackaged, and that can sometimes be eaten raw.

The terms “free” or “substantially free” refer to the total absence or near total absence of a particular compound in a composition, mixture, or ingredient.

The term “health care surface” refers to a surface of a surface of an instrument, a device, a cart, a cage, furniture, a structure, a building, or the like that is employed as part of a health care activity. Examples of health care surfaces include surfaces of medical or dental instruments, of medical or dental devices, of electronic apparatus employed for monitoring patient health, and of floors, walls, or fixtures of structures in which health care occurs. Health care surfaces are found in hospital, surgical, infirmity, birthing, mortuary, nursing home, and clinical diagnosis rooms. These surfaces can be those typified as “hard surfaces” (such as walls, floors, bed-pans, etc.), or fabric surfaces, e.g., knit, woven, and non-woven surfaces (such as surgical garments, draperies, bed linens, bandages, etc.), or patient-care equipment (such as respirators, diagnostic equipment, shunts, body scopes, wheel chairs, beds, etc.), or surgical and diagnostic equipment. Health care surfaces include articles and surfaces employed in animal health care.

The term “instrument” refers to the various medical or dental instruments or devices that can benefit from cleaning with a composition according to the present invention. As used herein, the phrases “medical instrument,” “dental instrument,” “medical device,” “dental device,” “medical equipment,” or “dental equipment” refer to instruments, devices, tools, appliances, apparatus, and equipment used in medicine or dentistry. Such instruments, devices, and equipment can be cold sterilized, soaked or washed and then heat sterilized, or otherwise benefit from cleaning in a composition of the present invention. These various instruments, devices and equipment include, but are not limited to: diagnostic instruments, trays, pans, holders, racks, forceps, scissors, shears, saws (e.g. bone saws and their blades), hemostats, knives, chisels, rongeurs, files, nippers, drills, drill bits, rasps, burrs, spreaders, breakers, elevators, clamps, needle holders, carriers, clips, hooks, gouges, curettes, retractors, straightener, punches, extractors, scoops, keratomes, spatulas, expressors, trocars, dilators, cages, glassware, tubing, catheters, cannulas, plugs, stents, scopes (e.g., endoscopes, stethoscopes, and arthoscopes) and related equipment, and the like, or combinations thereof.

The term “Internet” refers to the global system of interconnected computer networks that use the Internet protocol suite (TCP/IP) to link devices worldwide. It is a network of networks that consists of private, public, academic, business, and government networks of local to global scope, linked by a broad array of electronic, wireless, and optical networking technologies. The Internet carries a vast range of information resources and services, such as the inter-linked hypertext documents and applications of the World Wide Web (WWW), electronic mail, telephony, and file sharing. Consequently, the term, “Internet-based IoT,” refers to an Internet of Things (IoT) that has the capability of electronically communicating via the Internet with a sequential application and delivery system, with particular devices and sensors within the sequential application and delivery system, and/or users located inside or outside of the volumetric space.

The term “intranet” refers to a private network accessible only to an organization's staff. A wide range of information and services from the organization's internal Information Technology (IT) systems are generally available that would not be available to the public from the Internet. A company-wide intranet can constitute and important focal point of internal communication and collaboration, and provide a single starting point to access internal and external resources. In its simplest form, an intranet is established with technologies for local area networks (LANs) and wide area networks (WANs). Consequently, the term, “intranet-based IoT,” refers to an IoT that has the capability of electronically communicating via an intranet with a sequential application and delivery system, with particular devices and sensors within the sequential application and delivery system, and/or users located inside or outside of the volumetric space.

The term “liquid composition” refers to a combination of liquid components. Although in several embodiments, a liquid composition can comprise water and the term “liquid composition” is synonymous with an “aqueous composition,” liquid compositions can comprising non-aqueous compositions, including but not limited to oil-based compositions; organic compounds, solvents, or compositions, and other volatile compounds or compositions that are substantially free of water.

The term “microorganism” refers to any noncellular or unicellular (including colonial) organism. Microorganisms include all prokaryotes. Microorganisms include bacteria (including cyanobacteria), spores, lichens, fungi, protozoa, virinos, viroids, viruses, phages, and some algae. As used herein, the term “microbe” is synonymous with microorganism.

The phrase “organic acid compound” refers to any acid that is capable of forming a peracid that is effective as a disinfecting agent.

The terms “peracid” or “peroxyacid” refer to any acid having the hydrogen of a hydroxyl group replaced by a perhydroxyl group. Oxidizing peracids are referred herein as peroxycarboxylic acids.

The phrase “peracid reactant compound” refers to a reactant compound that will react to form a peracid on the target surface in situ.

The term “peroxide compound” refers to any compound that can react with an organic acid to form a peracid, including but not limited to hydrogen peroxide, metal peroxides, and ozone.

The term “polyhydric alcohol” refers to an alcohol that has two or more hydroxyl groups. Polyhydric alcohols suitable for use in the aqueous compositions include but are not limited to sugars, sugar alcohols, and non-aliphatic polyhydric alcohols such as phenols.

The term “reaction layer” refers to a layer formed on a surface to be disinfected, when a second aqueous composition comprising a second peracid reacting compound is deposited onto a coalesced first aqueous composition layer comprising a first peracid reactant compound formed on the surface. The peracid product of the two reactant compounds is formed in situ on the reaction layer.

The term “sprayer” refers to any device that is configured to dispense an aqueous composition into a volumetric space or onto a surface. Non-limiting examples of “sprayers” include traditional fogging devices, such as Hurricane™ sprayers, provided by Curtis Dyna-Fog, Ltd., but also other dispensing devices such as vaporizers and mechanical coarse spray devices, such as sprinkler systems that are capable of dispensing aqueous compositions as a jet, mist, or liquid stream.

The term “vapor” refers to a fluid phase or state in which a portion of an aqueous composition is substantially entirely in a gaseous state, as opposed to other embodiments in which there are a significant portion of liquid microdroplets of the aqueous composition suspended in the air.

The terms “weight percent,” “percent by weight,” “w/w,” and other variations, as used herein, refer to the concentration of a substance as a weight of that substance divided by the total weight of the composition, multiplied by 100. It is understood that “percent,” “%,” and like terms are intended to be synonymous with “weight percent,” “percent by weight,” etc, rather than percent by volume of the composition.

In describing embodiments of the disinfecting methods and system in the present disclosure, reference will be made to “first” or “second” as they refer to aqueous compositions or peracid reactant compounds. Except when there is clear context that a specific order is intended, “first” and “second” are merely relative terms, and a “first” composition or reactant compound described could just as easily and conveniently be referred to as a “second” composition or reactant compound, and such description is implicitly included herein.

Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight ratio range of about 0.5% to about 10% by weight includes not only the explicitly recited limits of 0.5% by weight and 10% by weight, but also individual weights such as 1% by weight and 5% by weight, and sub-ranges such as 2% to 8% by weight, 5% to 7% by weight, etc.

### Chemical Disinfection Methods

In accordance with these definitions, several methods are provided for disinfecting target surfaces within a volumetric space by using a peracid, particularly methods where reactant compounds capable of forming a peracid are dispersed sequentially onto those surfaces and the peracid is formed in situ directly on the surface. Additionally, this invention overcomes instability and safety issues associated with forming peracids prior to applying them onto a surface, as well as potential environmental and safety hazards related to utilizing peracids in disinfection as a whole.

While other sterilization methods attempt to solve the peracid stability and safety problem by including one or more additives in the reaction mixtures to promote the retention of the peracid in the system, many of these additives are expensive to produce and are not readily attainable for an average person with no connection to the chemical industry. In contrast, several embodiments of this invention harness the power of peracid chemistry to disinfect target surfaces while utilizing ingredients obtainable at a local grocery or department store that have a very long shelf life and that are universally regarded as safe. In such embodiments, aqueous compositions utilized in the disinfection methods of the present invention are substantially free of surfactants, polymers, chelators, and metal colloids or nanoparticles.

Without being limited by theory, it is believed that peracids are so effective as disinfectants because they are powerful oxidizing agents that can irreversibly damage proteins and DNA within microorganisms. Peracids are formed in an acid-catalyzed reaction when a strong oxidizing agent, such as a peroxide compound, comes into contact with an organic acid compound. For example, in a system that utilizes acetic acid as the organic acid compound, the addition of a peroxide compound such as hydrogen peroxide can result in a reaction in which peracetic acid and water are produced in equilibrium as shown in reaction (1) below:

H₂O₂+CH₃COOH↔CH₃COO—OH+H₂O (1).

Once the peracid is formed on the surface to be disinfected, it is strongly electrophilic. If there are no electron-rich sources in solution with the peracid, the excess water will drive equilibrium toward hydrolysis of the peracid and back into production of the parent acid. Additionally, as the parent acid becomes increasingly acidic, the resultant peracid similarly becomes more reactive. Thus, even though the resultant peracid could become an even better disinfectant under those conditions, it is also more unstable and likely to never reach the target surface, regardless of how immediately before application the individual components are mixed. Consequently, embodiments of this invention can similarly be more effective than the present art in industrial applications where stronger and more strictly-controlled components are used and cost is not an object.

The volumetric spaces in which the methods of the present invention can be performed are extraordinarily diverse, and can include volumetric spaces that are both accessible and inaccessible to humans and animals. Accessible volumetric spaces include spaces that are used to eat, work, sleep, and/or conduct other common activities associated with everyday life. Non-limiting examples include, but are not limited to: living spaces such as family rooms, bedrooms, kitchens, restrooms, basements, garages, and other rooms commonly found in one's home; classrooms; offices; retail spaces; hotel rooms; hospital patient rooms, operating rooms; food-operations spaces including dining, food preparation, packaging, and processing facilities; shipping containers; animal pens, factories and other industrial areas; and passenger compartments utilized in transportation, including personal vehicles, cabs, buses, subway and other rail cars, ferries, and airplanes. Non-limiting examples of surfaces in a hospital patient room that can be disinfected and sterilized include the wall, floor, bed frame, patient care equipment, bedside table, and bedding.

On the other hand, inaccessible volumetric spaces include, but are not limited to: heating, ventilation, and air conditioning (HVAC) systems; plumbing systems; liquid storage containers, and other compartments and spaces in which a human or animal cannot enter. Methods to disinfect surfaces in such inaccessible volumetric spaces include both clean-in-place (CIP) and clean-out-of-place (COP) procedures. For instance, surfaces within an HVAC or plumbing system can be disinfected using CIP methods, by dispensing compositions through an inlet in the HVAC or plumbing system. The HVAC or plumbing system can also be utilized as a carrier system to disinfect surfaces in which disinfecting equipment cannot access, such as, as a non-limiting example, utilizing the HVAC system of an automobile to disinfect surfaces in the passenger compartment, while the disinfecting equipment itself remains outside of the vehicle. In another non-limiting example, the first and second aqueous composition can be transported through the HVAC system of an airplane into the passenger cabin and other areas accessible to airline travelers.

Conversely, COP procedures can be utilized to disinfect contaminated surfaces of parts, components, and other equipment that can be disassembled from a larger machine or assembly. As a non-limiting example, parts used in industrial meat-packing equipment can be disassembled from the framework of a large machine and disinfected separately from the rest of the machine. In such methods, the parts can be placed on top of a surface situated in any of the volumetric spaces listed above, or inside a sealable tank, compartment, or housing, which once sealed, comprises the volumetric space.

Additionally, disinfectant compositions described in methods of the present invention can be applied to a variety of hard or soft surfaces having smooth, irregular, or porous topography. Suitable hard or and/or non-porous surfaces include, for example, architectural surfaces (e.g., floors, walls, windows, sinks, tables, counters and signs); eating utensils; hard-surface medical or surgical instruments and devices; and hard-surface packaging constructed from materials including, but not limited to plastics; metals; Linoleum; tiles; vinyl; stone; wood; concrete; glass; and vinyl. Suitable soft and/or porous surfaces include, for example, wallboards; plaster; pulp and fiber-based materials; paper; filter media, hospital and surgical linens and garments; soft-surface medical or surgical instruments and devices; and soft-surface packaging. Such soft surfaces can be made from a variety of materials including, for example, paper, fiber, woven or nonwoven fabric, soft plastics and elastomers.

In a first embodiment of this invention, a method to disinfect a surface in need of disinfecting within a volumetric space is provided, comprising the steps of: a) dispensing onto the surface a first aqueous composition comprising a first peracid reactant compound that is either a peroxide compound or an organic acid compound capable of reacting with a peroxide compound to form a peracid; b) allowing a time sufficient for the first aqueous composition to distribute across the surface and coalesce into a first aqueous composition layer upon the surface; c) dispensing onto the surface a second aqueous composition comprising a second peracid reactant compound that is the other of the first peracid reactant compound; and d) allowing a second time sufficient for the second aqueous composition to combine with the coalesced first aqueous composition layer and to form a reaction layer upon the surface, thereby forming a peracid in situ within the reaction layer and disinfecting the surface.

The first aqueous composition and the second aqueous composition can be dispensed into the volumetric space and/or onto surfaces to be disinfected using means commonly known to those skilled in the art, including but not limited to direct application using a mop, cloth, or sponge; streaming as a liquid stream from a hose or mechanical coarse spray device; or dispersing into the volumetric space as a multiplicity of microdroplets, including methods in which the multiplicity of microdroplets is formed when the aqueous compositions are dispersed as a vapor that has cooled and condensed into microdroplets. In some embodiments, a method for disinfecting a surface in need of disinfecting within a volumetric space as a multiplicity of microdroplets comprises the steps of: a) dispersing into the volumetric space a multiplicity of droplets of a first aqueous composition comprising a first peracid reactant compound that is either a peroxide compound or an organic acid compound capable of reacting with a peroxide compound to form a peracid; b) allowing a time sufficient for the first aqueous composition to distribute throughout the volumetric space, and to deposit and coalesce into a layer upon the surface; c) dispersing into the volumetric space a multiplicity of droplets of a second aqueous composition comprising a second peracid reactant compound that is the other of the first peracid reactant compound; and d) allowing a second time sufficient for the droplets of the second aqueous composition to deposit onto the coalesced layer of the first aqueous composition to form a reaction layer, thereby forming a peracid in situ on the reaction layer and disinfecting the surface.

As long as a peracid is formed only on the surface to be disinfected, the effectiveness of the methods described herein is expected to be independent of the order in which the peracid reactant compounds are dispersed. Thus, the first peracid reactant compound can either be an organic acid compound or a peroxide compound, so long as the second peracid reactant compound is the opposite compound of that chosen to be the first peracid reactant compound. For example, the second peracid reactant compound is an organic acid compound if a peroxide compound is selected to be the first peracid reactant compound, and the second peracid reactant compound is a peroxide compound if an organic acid compound is selected to be the first peracid reactant compound. Although the compositions containing the peracid reactant compounds are generally mostly aqueous, water need not comprise the majority of the composition. Furthermore, any liquid carrier system that can facilitate the formation of the peracid from a peroxide compound and an organic acid compound can be used.

Furthermore, the effectiveness of the methods described herein is also associated with ensuring that the first aqueous composition remains on the surface to be disinfected within the first aqueous composition layer until the second aqueous composition is deposited onto the surface. In some embodiments, substantially all of the first aqueous composition is retained on the surface upon dispensing the second aqueous composition onto the surface. Those skilled in the art would appreciate that retaining the first aqueous composition on the surface means that once applied to the surface, the first aqueous composition is not rinsed, wiped, or otherwise removed from the surface prior to dispensing the second aqueous composition on the surface.

The peroxide compound can be any compound that can react with an organic acid compound to form a peracid. Generally, these will include but not be limited to hydrogen peroxide, metal peroxides, or ozone. In some embodiments, an aqueous composition containing a peroxide compound comprises at least about 0.1% by weight of the peroxide compound, including at least about 0.5%, at least about 1%, at least about 2%, at least about 4%, at least about 6%, at least about 8%, at least about 10%, at least about 12%, at least about 14%, at least about 16%, at least about 18%, at least about 20%, or at least about 25% by weight of the peroxide compound. In other embodiments, an aqueous composition containing a peroxide compound comprises less than or equal to about 25% by weight of the peroxide compound, including less than or equal to about 20%, less than or equal to about 18%, less than or equal to about 16%, less than or equal to about 14%, less than or equal to about 12%, less than or equal to about 10%, less than or equal to about 8%, less than or equal to about 6%, less than or equal to about 4%, less than or equal to about 2%, less than or equal to about 1%, less than or equal to about 0.5%, or less than or equal to about 0.1% by weight of the peroxide compound. Useful ranges can be selected from any value between and inclusive of about 0.1% by weight to about 25% by weight of the peroxide compound. Non-limiting examples of such ranges can include from about 0.1% to about 25% by weight, from about 0.5% to about 25% by weight, from about 1% to about 25% by weight, from about 2% to about 25% by weight, from about 4% to about 25% by weight, from about 6% to about 25% by weight, from about 8% to about 25% by weight, from about 10% to about 25% by weight, from about 12% to about 25% by weight, from about 14% to about 25% by weight, from about 16% to about 25% by weight, from about 18% to about 25% by weight, from about 20% to about 25% by weight, from about 0.5% to about 20% by weight, from about 1% to about 18% by weight, from about 2% to about 16% by weight, from about 5% to about 15% by weight, or from about 7% to about 12% by weight of the peroxide compound. In some embodiments, the aqueous composition comprises about 10% by weight of the peroxide compound. In preferred embodiments, the peroxide compound is hydrogen peroxide.

The organic acid compound can be any organic acid that can effectively form a peracid upon reacting with a peroxide compound. Generally, these will include but not be limited to carboxylic acids. Non-limiting examples of carboxylic acids which can be used include formic acid, acetic acid, citric acid, succinic acid, oxalic acid, propanoic acid, lactic acid, butanoic acid, pentanoic acid, octanoic acid, amino acids, and mixtures thereof. In some embodiments, an aqueous composition containing an organic acid compound comprises at least about 0.5% by weight of the organic acid compound, including at least about 1%, at least about 2%, at least about 5%, at least about 10%, at least about 15%, at least about 20%, at least about 25%, at least about 30%, at least about 35%, at least about 40%, at least about 45%, or at least about 50% by weight of the organic acid compound. In other embodiments, an aqueous composition containing an organic acid compound comprises less than or equal to about 50% by weight of the organic acid compound, including less than or equal to about 45%, less than or equal to about 40%, less than or equal to about 35%, less than or equal to about 30%, less than or equal to about 25%, less than or equal to about 20%, less than or equal to about 15%, less than or equal to about 10%, less than or equal to about 5%, less than or equal to about 2%, less than or equal to about 1%, or less than or equal to about 0.5% by weight of the organic acid compound. Useful ranges can be selected from any value between and inclusive of about 0.5% to about 50% by weight of the organic acid compound. Non-limiting examples of such ranges can include from about 0.5% to about 50% by weight, from about 1% to about 50% by weight, from about 2% to about 50% by weight, from about 5% to about 50% by weight, from about 10% to about 50% by weight, from about 15% to about 50% by weight, from about 20% to about 50% by weight, from about 25% to about 50% by weight, from about 30% to about 50% by weight, from about 35% to about 50% by weight, from about 40% to about 50% by weight, from about 45% to about 50% by weight. from about 1% to about 35% by weight, from about 2% to about 20% by weight, or from about 4% to about 12% by weight of the organic acid compound. In some embodiments, the aqueous composition comprises about 10% by weight of the organic acid compound. In preferred embodiments, the organic acid compound is acetic acid.

As described above, the synthesis of peracids from an organic acid compound and a peroxide compound is an acid-catalyzed process (see Zhao, X., et al., (2007) Journal of Molecular Catalysis A 271:246-252). Typically, organic acids such as acetic acid and the others listed above have at least one carboxylate functional group with an acidic pKa value less than or equal to about 7, making such compounds suitable for reacting with a peroxide compound to produce a peracid. Some organic acids, such as citric acid, have multiple carboxylic acid groups which each have a pKa value below 7 and can thus react with a peroxide compound to form the peracid product. However, organic acids that possess carboxylic acid functional groups with pKa values above 7 can be used as also substrates so long as at least one of the carboxylic acid functional groups has a pKa value less than or equal to about 7. Consequently, in some embodiments, the pH of the composition comprising the organic acid compound is less than or equal to about 7. In further embodiments, the pH of the reaction layer is less than or equal to about 7.

In some embodiments, the first aqueous composition and/or the second aqueous composition are each dispensed as a liquid stream on the surface. In further embodiments, the method further comprises the step of providing a mechanical coarse spray device, wherein the first aqueous composition and/or the second aqueous composition are dispensed as a liquid stream onto the surface using the mechanical coarse spray device; particularly wherein the liquid stream is dispensed in the form of a mist, a shower, or a jet. Non-limiting examples of such mechanical coarse spray devices include spray nozzles and sprinkler systems that are capable of dispersing aqueous compositions as liquid streams and/or macrodroplets having an effective diameter of 100 microns or larger. In even further embodiments, the macrodroplets have an effective diameter at least about 100 microns, including at least about 250 microns, 500 microns, 1 millimeter, 2 millimeters, 3 millimeters, or 4 millimeters, and up to about 5 millimeters, including up to about 4 millimeters, 3 millimeters, 2 millimeters, 1 millimeter, 500 microns, or 250 microns. In still even further embodiments, at least about 90 percent of the multiplicity of microdroplets, including about 95 or 98 percent, up to about 99 percent, of the multiplicity of microdroplets has an effective diameter of at least about 100 microns, including at least about 250 microns, 500 microns, 1 millimeter, 2 millimeters, 3 millimeters, or 4 millimeters, and up to about 5 millimeters, including up to about 4 millimeters, 3 millimeters, 2 millimeters, 1 millimeter, 500 microns, or 250 microns.

Dispensing the first aqueous composition and the second aqueous composition as a liquid stream can be advantageous when disinfecting non-porous surfaces that are not sensitive to the amount of liquid placed on them, when only one or a small number of surfaces need to be disinfected relative to the number of surfaces within or the size of the volumetric space, or when the surface or surfaces can be dried manually after the peracid has been formed on the surface and the surface is disinfected. In particular, a liquid stream can be used in flood recovery and moisture remediation to disinfect contaminated non-porous surfaces and building materials that remain after all of unsalvageable soft or porous materials have been removed. Such non-porous surfaces and building materials can include, but are not limited to, metal, glass, certain tiles, and hard plastics.

Similarly, methods in which only a selected number of surfaces within a volumetric space are to be disinfected can be accomplished while avoiding contacting other surfaces within the volumetric space with either aqueous composition. As a non-limiting example, a user can utilize a hand-held mechanical coarse spray device to selectively dispense or apply the first aqueous composition onto a surface, and after allowing a time sufficient for the first aqueous composition to distribute across the surface and coalesce into a first aqueous composition layer upon the surface, the user can dispense or apply the second aqueous composition onto the first aqueous composition layer using a hand-held mechanical coarse spray device. In another non-limiting example, the first aqueous composition and the second aqueous composition can be dispensed through a mounted, overhead sprinkler system into a volumetric space and onto surface(s) below. In a further embodiment, surfaces to be disinfected using an overhead sprinkler system can include food and/or food-contact surfaces.

In some embodiments, at least about 90, 95, 97, 98, or 99 percent of the aqueous compositions are dispersed into the volumetric space and onto to the surface(s) to be disinfected as a multiplicity of microdroplets. In further embodiments, essentially 100 percent of the aqueous compositions are dispersed as a multiplicity of microdroplets. As defined above, microdroplets have an effective diameter of less than 100 microns. In such embodiments, the method to disinfect a surface within a volumetric space can comprise the steps of a) dispersing into the volumetric space a multiplicity of microdroplets of a first aqueous composition comprising a first peracid reactant compound that is either a peroxide compound or an organic acid compound capable of reacting with a peroxide compound to form a peracid; b) allowing a time sufficient for the multiplicity of microdroplets of the first aqueous composition to distribute throughout the volumetric space and to deposit and coalesce into a first aqueous composition layer upon the surface; c) dispersing into the volumetric space a multiplicity of microdroplets of a second aqueous composition comprising a second peracid reactant compound that is the other of the first peracid reactant compound; and d) allowing a second time sufficient for the multiplicity of microdroplets of the second aqueous composition to deposit onto the coalesced first aqueous composition layer to form a reaction layer upon the surface, thereby forming a peracid in situ within the reaction layer and disinfecting the surface.

The time sufficient for the multiplicity of microdroplets of each of the aqueous compositions to disperse into a volumetric space, and to deposit and coalesce into a layer upon the surface or surfaces to be disinfected, can depend on several factors, including but not limited to: the size and velocity of the microdroplets as they are dispersed; the volumetric size and humidity of the volumetric space; and the identity and concentration of the components within the aqueous composition. With regard to microdroplet size, the time sufficient for the microdroplets to reach and coalesce upon the surfaces to be disinfected is approximately inversely proportional to the size of the microdroplet. Thus, when a microdroplet is small, for example with an effective diameter of about 1 to about 2 microns, more time is needed for the microdroplet to deposit onto a surface than when microdroplet is large, for example with an effective diameter of about 50 to about 100 microns. Although these large microdroplet sizes are functionally adequate for disinfecting multiple surfaces in larger volumetric spaces such as rooms or shipping containers, it has been observed that the ability of the microdroplets to remain in the air long enough to overcome gravity and reach the surfaces to be disinfected becomes compromised once the effective diameter of the microdroplets reaches about 20 microns or more.

Accordingly, in some embodiments, the preponderance of the multiplicity of microdroplets have an effective diameter of at least about 1 micron, including at least about 5 microns, at least about 10 microns, at least about 15 microns, at least about 20 microns, at least about 25 microns, at least about 30 microns, at least about 35 microns, at least about 40 microns, at least about 45 microns, at least about 50 microns, at least about 60 microns, at least about 70 microns, at least about 80 microns, at least about 90 microns, or at least about 100 microns. In other embodiments, the preponderance of the multiplicity of microdroplets have an effective diameter of less than or equal to about 100 microns, including than or equal to about 90 microns, less than or equal to about 80 microns, less than or equal to about 70 microns, less than or equal to about 60 microns, less than or equal to about 50 microns, less than or equal to about 45 microns, less than or equal to about 40 microns, less than or equal to about 35 microns, less than or equal to about 30 microns, less than or equal to about 25 microns, less than or equal to about 20 microns, less than or equal to about 15 microns, less than or equal to about 10 microns, or less than or equal to about 5 microns. Useful ranges for the effective diameter of a preponderance of the multiplicity of microdroplets can be selected from any value between and inclusive of about 1 micron to about 100 microns. Non-limiting examples of such ranges can include from about 1 micron to about 100 microns, from about 5 microns to about 100 microns, from about 10 microns to about 100 microns, from about 15 microns to about 100 microns, from about 20 microns to about 100 microns, from about 25 microns to about 100 microns, from about 30 microns to about 100 microns, from about 35 microns to about 100 microns, from about 40 microns to about 100 microns, from about 45 microns to about 100 microns, from about 50 microns to about 100 microns, from about 60 microns to about 100 microns, from about 70 microns to about 100 microns, from about 80 microns to about 100 microns, from about 90 microns to about 100 microns, from 3 microns to about 75 microns, or from about 10 microns to about 25 microns. Spraying and fogging devices capable of dispersing a multiplicity of microdroplets having effective diameters fitting any of the above ranges are well known to those skilled in the art.

However, issues can also potentially arise when the effective diameter of the microdroplets is small. It is known that airborne microdroplets can be inhaled and retained in the deep lung at effective diameters less than about 8 to about 10 microns, as illustrated in Drug and Biological Development: From Molecule to Product and Beyond, edited by Ronald Evens, pg. 210 and applicable sections, 2007, hereby incorporated by reference in its entirety. Consequently, although humans and animals should not be present in a volumetric space without adequate protection during the dispensing of the aqueous compositions, in some embodiments of the invention where a person is present in the area or volumetric space while either aqueous composition is dispersed in microdroplet form, the minimum effective diameter of substantially all of the microdroplets should remain above about 10 microns, in order to minimize and avoid deep lung penetration. Accordingly, in some embodiments, the minimum effective diameter of the multiplicity of microdroplets dispersed of an aqueous composition is about 15 microns. In other embodiments where a person is not present in the room when the aqueous compositions are dispersed, the minimum effective diameter of the multiplicity of microdroplets can be any diameter that facilitates distribution, deposition, and coalescence of the microdroplets onto a surface or surfaces to be disinfected, including such effective diameters as listed above.

In some embodiments, once the multiplicity of microdroplets of the first aqueous composition is deposited onto a surface to be disinfected, the microdroplets preferably coalesce into a layer having a substantially uniform thickness, in order to provide maximal coverage on the surface. In preferred embodiments, the actual deposited thickness of the coalesced layer should be minimized while also substantially covering and coating the entire surface in all exposed and unexposed locations. The thickness of the coalesced layer is dependent on both the size and surface tension of the multiplicity of microdroplets. In some embodiments where the multiplicity of microdroplets consists only of peroxide compounds or organic acid compounds in an aqueous solution, the microdroplets can possess a surface tension close to that of pure water, which is about 72 dyne/cm at 20° C. In this situation, the coalesced layer may be thicker because the microdroplets will narrowly spread after being deposited upon the surface. Thus, more composition is needed to completely cover the entire area of the surface, to disinfect the entire surface. Conversely, the multiplicity of microdroplets may additionally include non-aqueous compounds that lower the composition's surface tension. For example, pure ethanol has a surface tension of about 22.27 dyne/cm at 20° C. In this situation, the composition microdroplets with the lower surface tension will more widely spread over the surface, creating a thinner coalesced layer that requires less of the composition to completely cover the entire area of the surface, to disinfect the entire surface.

Thus, in some embodiments, the coalesced layer can have an effective uniform thickness, and preferably an actual uniform thickness, of at least about 1 micron, including at least about 2 microns, at least about 3 microns, at least about 5 microns, at least about 8 microns, at least about 10 microns, at least about 15 microns, or at least about 20 microns. In other embodiments, the coalesced layer can have an effective uniform thickness, and preferably an actual uniform thickness, of less than or equal to about 20 microns, including less than or equal to about 15 microns, less than or equal to about 10 microns, less than or equal to about 8 microns, less than or equal to about 5 microns, less than or equal to about 3 microns, less than or equal to about 2 microns, or less than or equal to about 1 micron. Useful ranges for the substantially uniform thickness of a coalesced layer of an aqueous composition can be selected from any value between and inclusive of about 1 micron to about 20 microns. Non-limiting examples of such ranges can include from about 1 micron to about 20 microns, from about 2 microns to about 20 microns, from about 3 microns to about 20 microns, from about 5 microns to about 20 microns, from about 8 microns to about 20 microns, from about 10 microns to about 20 microns, from about 15 microns to about 20 microns, or from about 3 microns to about 8 microns.

In some embodiments, an alcohol can be further comprised within one or both of the aqueous compositions to decrease the surface tension of the compositions deposited on the surface to be disinfected. Om further embodiments, an alcohol can be further comprised within aqueous composition dispersed as microdroplets. The alcohol contained in either aqueous composition promotes a thinner coalesced layer without having to reduce the microdroplet size to a smaller effective diameter, where a sufficiently small diameter could potentially result in deep lung penetration for any persons or animals in the area or volumetric space. Furthermore, some alcohols also independently provide biocidal activity separate from the peracid. Therefore, using alcohols in combination with forming the peracid in situ on the surface to be disinfected may provide additive effects on the antimicrobial activity as compared to reaction layers which only contain a peroxide compound and an organic acid compound.

Although an alcohol in liquid form can be used at high concentrations (70% by weight or above) to sterilize instruments or surfaces, the lowest molecular weight alcohols may be combustible at those same concentrations when volatilized, especially as the temperature of the area or volumetric space is increased. Thus, in some embodiments, an aqueous composition comprising an alcohol can comprise at least about 0.05% by weight of the alcohol, including at least about 0.1, 1, 5, 10, 15, 20, 25, 30, 40, 50, 60, or 70% by weight of the alcohol. In other embodiments, an aqueous composition containing an alcohol comprises less than or equal to about 0.05% by weight of the alcohol, including less than or equal to about 0.1, 1, 5, 10, 15, 20, 25, 30, 40, 50, 60, or 70% by weight of the alcohol. Useful ranges can be selected from any value between and inclusive of about 0.05% to about 70% by weight of the alcohol. Non-limiting examples of such ranges can include from about 0.05% to about 70% by weight, from about 0.1% to about 70% by weight, from about 1% to about 70% by weight, from about 5% to about 70% by weight, from about 10% to about 70% by weight, from about 15% to about 75% by weight, from about 20% to about 70% by weight, from about 25% to about 70% by weight, from about 30% to about 70% by weight, from about 40% to about 70% by weight, from about 50% to about 70% by weight, from about 60% to about 70% by weight, from about 1% to about 25% by weight, or from about or 10% to about 20% by weight of the alcohol. In some embodiments, an aqueous composition comprising an alcohol can comprise about 15% by weight of the alcohol. In other embodiments, an aqueous composition comprising an alcohol can comprise about 5% by weight of the alcohol.

The alcohol present in an aqueous composition can be a single alcohol or a combination of multiple alcohols. An alcohol can include aliphatic alcohols and other carbon-containing alcohols having from 1 to 24 carbons. The alcohol can be selected from a straight-chained or completely saturated alcohol or other carbon-containing alcohols, including branched aliphatic alcohols, alicyclic, aromatic, and unsaturated alcohols. Polyhydric alcohols can also be used alone or in combination with other alcohols. Non-limiting examples of polyhydric alcohols which can be used in the present disclosure include ethylene glycol (ethane-1,2-diol) glycerin (or glycerol, propane-1,2,3-triol), propane-1,2-diol, polyvinyl alcohol, sorbitol, other polyols, and the like. Other non-aliphatic alcohols may also be used including but not limited to phenols and substituted phenols, erucyl alcohol, ricinolyl alcohol, arachidyl alcohol, capryl alcohol, capric alcohol, behenyl alcohol, lauryl alcohol (1-dodecanol), myristyl alcohol (1-tetradecanol), cetyl (or palmityl) alcohol (1-hexadecanol), stearyl alcohol (1-octadecanol), isostearyl alcohol, oleyl alcohol (cis-9-octadecen-1-ol), palmitoleyl alcohol, linoleyl alcohol (9Z, 12Z-octadecadien-1-ol), elaidyl alcohol (9E-octadecen-1-ol), elaidolinoleyl alcohol (9E, 12E-octadecadien-1-ol), linolenyl alcohol (9Z, 12Z, 15Z-octadecatrien-1-ol), elaidolinolenyl alcohol (9E, 12E, 15-E-octadecatrien-1-ol), and combinations thereof.

In some embodiments, for practical considerations, methanol, ethanol, isopropanol, propanol, tert-butanol, butanol, pentanol, hexanol, heptanol, octanol, nonanol, and decanol, including all constitutional isomers, stereoisomers, and denatured alcohols thereof, can be used because of their properties and cost. The alcohol can be selected to satisfy the requirements for food-grade and food-safe systems. However, many alcohols, particularly primary alcohols, for example methanol and ethanol, can form an ester in a side reaction with an organic acid compound. As a non-limiting example, ethanol and acetic acid can form ethyl acetate at room temperature, particularly under acidic pH conditions. Consequently, in preferred embodiments, isopropanol and t-butanol can be chosen because side reactions with the organic acid compound are not favored because isopropanol and t-butanol are secondary and tertiary alcohols, respectively.

In some embodiments, alcohols with four or more carbon atoms, including but not limited to C4-, C5-, C6-, C7-, C8-, C9-, and C10 alcohols, can be utilized because they have a relatively low vapor pressure, a relatively high flash point, and can reduce the surface tension of the coalesced layer and/or reaction layers on the surfaces at relatively low concentrations. In one non-limiting example, the surface tension of an aqueous solution with 15% (v/v) ethanol is about 33 dyne/cm at 20° C., whereas an aqueous solution with about 0.5% (v/v) of 1-hexanol has a surface tension of lower than 30 dyne/cm at 20° C. Furthermore, the flash points of pure C4-, C5-, C6-, C7-, C8-, C9-, and C10 alcohols are much higher than a standard room temperature of 20° C., and can safely be utilized within any of the aqueous compositions of the present invention when dispersing them into the volumetric space.

In other embodiments, additional compounds can be included in either aqueous composition to enhance or supplement the effectiveness of the peracid generated in situ on the surface to be disinfected. Such compounds can include one or more natural biocides, such as manuka honey and essential oils, and/or natural biocidal compounds typically found within manuka honey and essential oils, such as methylglyoxal, carvacrol, eugenol, linalool, thymol, p-cymene, myrcene, borneol, camphor, caryophillin, cinnamaldehyde, geraniol, nerol, citronellol, and menthol, including combinations thereof. Honey, particularly manuka honey, has long been known to have biocidal properties. The anti-bacterial properties of methylglyoxal, the primary component of manuka honey, has been described previously (see Hayashi, K., et al., (April 2014) Frontiers in Microbiology, 5 (180):1-6, hereby incorporated by reference in its entirety). Methylglyoxal has been shown to be effective against multidrug resistant bacteria, including methicillin-resistant Staphylococcus aureus (MRSA), multidrug-resistant Pseudomonas aeruginosa, and pathogenic Escherichia coli with minimum inhibitory concentrations (MIC) as low as 0.005% by weight of a composition.

In other embodiments, essential oils can be included in either aqueous composition. Essential oils have been widely-used in medicines throughout human history, and are particularly known to have antimicrobial activity at concentrations as low as 0.001% by weight, as described in Effect of Essential Oils on Pathogenic Bacteria, Pharmaceuticals, pg. 1451-1474, Volume 6, 2013, and Antimicrobial Activity of Some Essential Oils Against Microorganisms Deteriorating Fruit Juices, Mycobiology, pgs. 219-229, Volume 34, 2006, both of which are hereby incorporated by reference in their entirety. The use of essential oils as components in disinfectants is described in U.S. Pat. No. 6,436,342, the disclosure of which is incorporated by reference in its entirety. Non-limiting examples of essential oils that can be included in one or more of the aqueous compositions include the essential oils of oregano, thyme, lemongrass, lemons, oranges, anise, cloves, aniseed, cinnamon, geraniums, roses, mint, peppermint, lavender, citronella, eucalyptus, sandalwood, cedar, rosmarin, pine, vervain fleagrass, and ratanhiae.

In addition to their antimicrobial properties, several essential oils produce odors that are pleasing to subsequent users of the disinfected room or volumetric space after the method has been completed. Accordingly, one or more natural biocides or natural biocidal compounds, particularly essential oils and/or their chemical components, can be included in an aqueous composition at a concentration less than the MIC. Thus, in some embodiments, an aqueous composition can comprise one or more natural biocides or natural biocidal compounds at a concentration of at least about 0.001% by weight of the aqueous composition, including at least about 0.005, 0.01, 0.05, 0.1, 0.25, 0.5, or 1% by weight of the aqueous composition. In other embodiments, an aqueous composition can comprise one or more natural biocides or natural biocidal compounds at a concentration of less than or equal to about 0.001% by weight of the aqueous composition, including less than or equal to about 0.005, 0.01, 0.05, 0.1, 0.25, 0.5, or 1% by weight of the aqueous composition. Useful ranges can be selected from any value between and inclusive of about 0.001% to about 1% by weight of the natural biocide or natural biocidal compound within the aqueous composition. Non-limiting examples of such ranges can include from about 0.001% to about 1% by weight, from about 0.005% to about 1% by weight, from about 0.01% to about 1% by weight, from about 0.05% to about 1% by weight, from about 0.1% to about 1% by weight, from about 0.25% to about 1% by weight, from about 0.5% to about 1% by weight, from about 0.01% to about 0.5% by weight, or from about 0.06% to about 0.3% by weight of the natural biocide or natural biocidal compound within the aqueous composition.

Without being bound by a particular theory, the effective uniform thickness of a coalesced liquid layer or reaction layer can be optimized according to the desired concentrations of the peracid reactant compounds or any other component of the aqueous compositions. In other embodiments, the concentrations of the peracid reactant compounds or other components can be optimized according to the desired effective uniform thickness. For instance, in some embodiments in which the concentration of the peracid reactant compounds or other reaction components are desired to be relatively dilute, then the volume of the aqueous compositions dispersed can be adjusted accordingly in order to increase the effective uniform thickness of the reaction layer (thus, the total amount of peracid reactant compound present) and achieve a desired microbial kill. Such an embodiment can be useful in situations in which stock solutions used to form one or more of the aqueous compositions are less concentrated, as with acetic acid or hydrogen peroxide that can be purchased by consumers at their local grocery store or pharmacy. Conversely, in other embodiments in which industrial-grade stock solutions are available, a relatively higher peracid reactant concentration is desired, or the volumetric space is relatively large, the volume of the dispersed aqueous compositions can be adjusted in order to form a relatively thinner reaction layer. Those skilled in the art possess the requisite knowledge to determine the concentration of the peracid reactant compounds or other components to determine the volume of the aqueous compositions to disperse to form a reaction layer with a desired effective uniform thickness, based on factors such as the concentration of stock solutions, desired microbial kill, and the volume inside the volumetric space, among other factors.

An advantage of the components described above, including the peracid reactant compounds, alcohols, and natural biocidal compounds, is that they are easily volatilized after the sterilization is complete. Such embodiments include situations in which high turnover is required in order to enable people to return to the volumetric space as quickly as possible after the sterilization method is completed. In embodiments where the coalesced layer on the surfaces to be disinfected has an effective uniform thickness of about 1 micron to about 20 microns, the aqueous compositions can rapidly evaporate from treated surfaces, obviating the need for additional treatments to remove unwanted components and waste products, and facilitating a faster turnover of the area in which the surfaces are located. Accordingly, such embodiments require that non-volatile salts and high-molecular weight materials be used sparingly or omitted completely in order to promote high turnover of the volumetric space containing the surfaces to be disinfected. In some embodiments, the aqueous compositions have a volatility such that at least about 90% by weight of the reaction layer, including at least about 95%, at least about 99%, at least about 99.5%, at least about 99.7%, or at least about 99.9% by weight of the reaction layer can evaporate within 30 minutes of being formed.

To enhance the volatility of the aqueous compositions after they are deposited on one or more surfaces, the individual components of each of the aqueous compositions can be selected to have a relatively higher standard vapor pressure compared to less labile components that remain on surfaces long after they are disinfected. The standard vapor pressures of several typical components of the aqueous compositions are listed below in Table 1. It is noted that hydrogen peroxide on the surface that has not reacted with the organic acid compound would subsequently decompose into water and oxygen gas, each of which is much more volatile than hydrogen peroxide itself.

TABLE 1
Standard Vapor Pressures of Common
Aqueous Composition Components at 20° C.

| Compound Name | Vapor Pressure (mm Hg) |
|---|---|
| Water | 17.5 |
| Acetic Acid | 11.3 |
| Hydrogen Peroxide | 1.5 |
| Ethanol | 43.7 |
| Isopropanol | 44.0 |
| t-Butanol | 31.0 |
| 1-Butanol | 31.1 |
| 1-Pentanol | 24.9 |
| 1-Hexanol | 19.9 |
| 1-Heptanol | 15.9 |
| 1-Octanol | 12.7 |
| 1-Nonanol | 10.2 |
| 1-Decanol | 8.2 |

Thus, in some embodiments, one or both of the aqueous compositions can be formulated so at least about 99.0% by weight of the components, or at least about 99.5%, or at least about 99.9% by weight of the components within the aqueous composition have a standard vapor pressure of at least 1.0 mm Hg at 20° C. In further embodiments, one or both of the aqueous compositions can be formulated so that essentially 100% of the components by weight of the aqueous composition have a vapor pressure of at least about 1.0 mm Hg at 20° C.

Dispersing the first aqueous composition and the second aqueous composition as a multiplicity of microdroplets is particularly useful for disinfecting a wider range of materials, including materials that can become damaged after being contacted with large volumes of liquids. In one non-limiting example, water- or flood-damaged porous and semi-porous materials, such as drywalls, carpets, insulation, ceiling tiles, wood, and concrete, that can be dried and made salvageable can be disinfected by dispersing aqueous compositions as a multiplicity of microdroplets and forming microns-thick reaction layer on the surface, particularly where the components that comprise the aqueous compositions are volatile and will readily evaporate after the surface has been disinfected.

As stated above, in some embodiments of the invention, one or more aqueous compositions are substantially free of surfactants, polymers, chelators, and metal colloids or nanoparticles, and can particularly comprise only food-grade components. In other embodiments, however, it can be advantageous to include chemical stabilizers or enhancers in at least one of the aqueous compositions in order to compliment the disinfection of surfaces within a volumetric space, particularly in situations in which the volatility of the aqueous compositions once they have been deposited onto surfaces is not a concern. Such chemical stabilizers or enhancers can include, but are not limited to: surfactants, polymers, chelators, metal colloids and/or nanoparticles, oxidizers, and other chemical additives, including combinations thereof, the use of which is described in U.S. Pat. Nos. 6,692,694, 7,351,684, 7,473,675, 7,534,756, 8,110,538, 8,679,527, 8,716,339, 8,772,218, 8,789,716, 8,987,331, 9,044,403, 9,192,909, 9,241,483, and 9,540,248, as well as U.S. Patent Publications 2008/0000931; 2013/0199539; 2014/0178249; 2014/0238445; 2014/0275267; and 2014/0328949, the disclosures of which are incorporated by reference in their entireties.

In some embodiments, one or more chemical stabilizers or enhancers, such as the surfactants, polymers, chelators, metal colloids and/or nanoparticles, oxidizers, and other chemical additives described above, can be delivered or dispersed within one or more aqueous compositions in addition to the first or second aqueous compositions as described above that contain peracid reactant compounds.

Similarly, one or more supplemental aqueous compositions can be dispersed into the volumetric space in addition to the first aqueous composition and the second aqueous composition, which contain the peracid reactant compounds. Thus, over the course of a single treatment, three or more aqueous compositions can be utilized and dispersed according to the methods of the present invention. Accordingly, within such embodiments, peracid reactant compounds can be delivered by any two separate aqueous compositions dispersed during methods, and do not necessarily have to be included in the “first” or “second” aqueous composition dispersed so long as a peroxide compound and an organic acid compound are dispersed as part of two separate compositions and a peracid is formed in situ on a surface to be disinfected.

Thus, in some embodiments, methods of disinfecting a surface within a volumetric space can comprise the steps of: a) dispersing into the volumetric space a multiplicity of microdroplets of a first aqueous composition comprising a first peracid reactant compound that is either a peroxide compound or an organic acid compound capable of reacting with a peroxide compound to form a peracid; b) allowing a time sufficient for the multiplicity of microdroplets of the first aqueous composition to distribute throughout the volumetric space and to deposit and coalesce into a first aqueous composition layer upon the surface; c) dispersing into the volumetric space a multiplicity of microdroplets of a second aqueous composition comprising a second peracid reactant compound that is the other of the first peracid reactant compound; and d) allowing a second time sufficient for the multiplicity of microdroplets of the second aqueous composition to deposit onto the coalesced first aqueous composition layer to form a reaction layer upon the surface, thereby forming a peracid in situ within the reaction layer and disinfecting the surface, wherein the method further includes the steps of dispersing into the volumetric space one or more supplemental aqueous compositions and allowing a time sufficient for each dispersed supplemental aqueous composition to distribute throughout the volumetric space and to deposit onto the surface. Consequently, a supplemental aqueous composition can be dispersed into the volumetric space prior to dispersing the first aqueous composition into the volumetric space, after the first aqueous composition layer is formed upon the surface and prior to dispersing the second aqueous composition into the volumetric space, or after the peracid has been formed in situ within the reaction layer on the surface, including combinations thereof.

Similar to the first aqueous composition and the second aqueous composition, supplemental aqueous compositions can be directly applied to the surface using a mop, cloth, or sponge; streamed onto the surface as a liquid stream from a hose or mechanical coarse spray device; or dispersed into the volumetric space as a multiplicity of microdroplets, including methods in which the multiplicity of microdroplets is formed when the aqueous compositions are dispersed as a vapor that has cooled and condensed into microdroplets.

In some embodiments, the identity of a supplemental aqueous composition can be selected from the group consisting of a peracid scavenging composition, a pesticide composition, and an environmental conditioning composition.

Peracid scavenging compositions include components that can reduce or eliminate any excess peracids lingering on the surface(s) after the surface(s) have been disinfected. In some embodiments, the peracid scavenging composition comprises a metal halide compound and is dispersed after the peracid has been formed in situ within the reaction layer on the surface, wherein the metal halide compound comprises iodide, bromide, or chloride, particularly a metal halide compound selected from the group consisting of potassium iodide, potassium chloride, and sodium chloride, and more particularly potassium iodide. In other embodiments, the dispersion of a peracid scavenging composition after the peracid has been formed on the surface(s) to be disinfected can mitigate the number of air exchanges necessary to return the volumetric space to a habitable state and allow people to enter. As a non-limiting example, a peracid scavenging composition can be dispersed into the volumetric space as a final step to neutralize and remove lingering microdroplets that may be present within the volumetric space when the first aqueous composition and the second aqueous composition are dispersed as a vapor.

In aqueous systems, halide ions are known to react with peracids, particularly peracetic acid, to form a variety of products (see Shah, A. D., et al., (2015) Environmental Science & Technology 49:1698-1705). As is observed in Shah, the most common reaction in aqueous solutions is the reaction to form an acid, acetate, and water. The chemical reaction between peracetic acid and an iodide ion to form hypoiodous acid is shown in reaction (2) below:

CH_{3C}(O)OOH+I⁻→HOI+CH₃COO⁻+H₂O (2),

where k=4.2×10² M⁻¹ s⁻¹ (literature value). Reactions with chloride or bromide ions form similar hypohalous acid products, hypochlorous acid (HOCl) and hypobromous acid (HOBr), respectively. However, the reaction between the peracid and the halide ion causes a complex equilibrium with several reactions going on simultaneously. For instance, in the presence of a peroxide, such as hydrogen peroxide, hypohalous acids rapidly dissociate to form the parent halide, oxygen, and water. The dissociation reaction for hypoiodous acid is shown in reaction (3) below:

HOI+HO₂⁻→I⁻+½O₂+H₂O (3),

where k=1×10¹⁰ M⁻¹ s⁻¹ (estimated). Furthermore, in the presence of an acid, a peroxide such as hydrogen peroxide can under ago a redox reaction with the halide ion directly. to form the diatomic halide. The reaction between hydrogen peroxide and iodide ions (see Sattsangi, P. D. (2011) Journal of Chemical Education 88 (2):184-188) is shown below:

2I⁻+H₂O₂+2H⁺→I₂+H₂O (4),

where k=8.9×10⁻³ M⁻¹ s⁻¹ (literature value).

At high enough concentrations, hypoiodous acids, particularly HOCl and HOBr, as well as the diatomic bromine, chlorine, or iodine can be toxic to humans or animals that come in contact with the compounds. However, as long as hydrogen peroxide and peracetic acid are present in the system, reactions (2) and (3) form a catalytic cycle, as shown in scheme (S1) below:

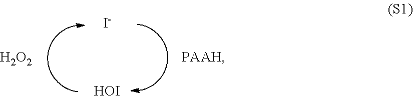

wherein PAAH is the acidic form of peracetic acid. Without being limited by a particular theory, it is believed that the catalytic cycle in scheme (S1) readily occurs in aqueous solutions rather than reaction (4) because of the rate constants for each reaction. The formation of I₂ in reaction (4) is disfavored because its rate constant indicates that the reaction approximately five orders of magnitude slower than reaction (2) and approximately 13 orders of magnitude slower than reaction (3). In embodiments in which the peroxide compound, particularly hydrogen peroxide, is added in excess of the organic acid compound, particularly acetic acid, the catalytic cycle will continue until all of the peracid has been scavenged, leaving the peroxide and the halide in solution until the solution evaporates or the surface is manually dried.

Historically, iodides have been used to assess the concentration of a peracid in a system, because the amount of iodine formed is proportional to the amount of the peracid in the system, as described in U.S. Pat. No. 3,485,588, the disclosure of which is incorporated by reference in its entirety. Potassium iodide is an extremely common source of iodide ions, and the concentration of potassium iodide that can be used to react with the peracid is effectively limited by its solubility in solution, and can be included in a solution in a concentration as high as 100 grams per 100 grams of water (equivalent to about 6 moles per liter). However, the use of high concentrations of potassium iodide can lead to unwanted residues from the formation of excess iodine or triiodide ions in solution. As a result, lower concentrations of potassium iodide can be utilized, including concentrations as low as 1 part per million (equivalent to about 1.87×10^{0.5} moles per liter), particularly because the process in scheme (S1) is catalytic and the iodide within the system is restored upon reaction of hypoiodous acid with hydrogen peroxide. Therefore, in some embodiments, the peracid scavenging composition comprises at least about 0.000001 moles per liter potassium iodide, including at least about 0.00001, 0.0001, 0.001, 0.01, 0.1, or 1 mole per liter potassium iodide, up to about 6 moles per liter potassium iodide. In other embodiments, a stoichiometric amount of the metal halide compound is dispersed that is equal to or greater than a stoichiometric amount of the peracid formed in situ within the reaction layer, thereby scavenging substantially all of the formed peracid from the surface.

Pesticide compositions can comprise any commercially available or synthesizable fungicide, rodenticide, herbicide, larvicide, or insecticide, including combinations thereof, particularly pesticides that can be applied by a liquid stream, as a multiplicity of microdroplets, or as a vapor. In some embodiments, included pesticides can provide, supplement, or enhance the activity of peracids generated in situ against pests, including but not limited to, parasites, insects, nematodes, mollusks, fungi, and rodents.

As a non-limiting example, one or more pesticides specific to the control and/or eradication of bed bugs or termites can be included in a pesticide composition. For bed bugs in particular, the Environmental Protection Agency has defined over 300 pesticide compounds within seven chemical classes, including pyrethrins, pyrethroids, pyrroles, neonicotinoids, desiccants, insect growth regulators, and other biochemical compounds. Pyrethrins and pyrethroids are the most common compounds used to control bed bugs and other indoor pests, and pyrethroids in particular have been shown to be effective when dispersed as droplets or vapors. However, some bed bug populations are resistant to pyrethrins and pyrethroids. In these situations, desiccants, pyrroles, neonicotinoids, and other biochemicals, including neem oil, have been shown to be effective against bed bugs because they operate using different physical and/or chemical modes of action. Non-limiting examples of desiccants include diatomaceous earth and boric acid. Insect growth regulators can be used in conjunction with or separately from the other classes of pesticides used against bed bugs, and operate not to necessarily kill a bed bug population but to either affect the bugs' ability to form their exoskeletons or by altering the bugs' development into adulthood.

Those skilled in the art can appreciate and identify compounds within a particular chemical class that are effective against a particular pestilent population, as well the measures necessary to protect users or bystanders from contact with such chemicals. In conjunction with spraying peracid reactant compounds and forming peracids on surfaces in situ, additionally dispersing one or more pesticides has the potential to effectively and powerfully eliminate substantially all pests, both micro- and macroscopic, from surfaces within an area. In some embodiments, the pesticide composition is dispersed into the volumetric space prior to dispersing the first aqueous composition into the volumetric space. In other embodiments, the pesticide composition is dispersed into the volumetric space after the peracid has been formed in situ within the reaction layer on the surface.

As a non-limiting example, an anti-bed bug pesticide composition can be dispersed in conjunction with methods of the present invention during the course of disinfecting a hotel room in between occupants. In some embodiments, the pesticide composition can comprise one or more compounds selected from the classes of compounds consisting of pyrethrins, pyrethroids, pyrroles, neonicotinoids, desiccants, insect growth regulators, and neem oil. In further embodiments, the pesticide composition comprises a pyrethrin or a pyrethroid.

Environmental conditioning compositions can be utilized in combination with dispersing the first aqueous composition and second aqueous composition for several applications, including preparation of the volumetric space for dispersing the first aqueous composition, the second aqueous compositions, or any of the other supplemental aqueous compositions; returning the volumetric space to a state where humans or animals can enter; and/or diluting the concentration of peracid on surfaces after they have been disinfected.

In some embodiments, an environmental conditioning composition consists essentially of water. Dispersing compositions consisting essentially of water opens up several optional possibilities with regard to pre-treatment, intermediate, and finishing steps that can be implemented in conjunction with the methods presented herein. For instance, in some embodiments, a method can further include the step of dispersing into the volumetric space prior to dispersing the first aqueous composition into the volumetric space. Dispersing the environmental conditioning composition prior to the first aqueous composition can increase the humidity in the volumetric space and inhibit or prevent the first or second aqueous composition from evaporating before the peracid reactant compounds can reach the surface to be disinfected. In some embodiments, the time sufficient for the environmental conditioning composition to distribute throughout the volumetric space is the time sufficient to cause the volumetric space to have a relative humidity of at least about 50 percent, including at least about 60, 70, 80, 90, or 95 percent, up to about 99 percent. In further embodiments, the time sufficient for the environmental conditioning composition to distribute throughout the volumetric space is the time sufficient to cause the volumetric space to have a relative humidity of at least about 90 percent. Those skilled in the art can determine the necessary volume of an environmental conditioning composition consisting of essentially of water to disperse in order to reach the desired relative humidity based on the atmospheric conditions within the volumetric space as well as the Cartesian dimensions of the volumetric space.

In other embodiments, the environmental conditioning composition can be dispersed into the volumetric space after the first aqueous composition layer is formed upon the surface and prior to dispersing the second aqueous composition into the volumetric space, in order to coalesce with and enhance deposition of any excess or lingering microdroplets of the first aqueous composition from the air. In another embodiment, the environmental conditioning composition can be dispersed into the volumetric space after the second aqueous composition has been dispersed, including after the peracid has been formed in situ on the surface, in order to coalesce with and enhance deposition of any excess or lingering microdroplets of the second aqueous composition in the volumetric space, or to dilute the peracid concentration on the surface after the surface has been disinfected. Removing excess or lingering suspended microdroplets of any aqueous composition containing a peracid reactant compound can render the volumetric space substantially free of any of the chemical components dispersed during disinfection.

Additionally, the environmental conditioning composition can further consist essentially of a fragrant compound, in order to leave the volumetric space with a pleasant odor. The fragrant compound can include one or more of the essential oils described above, such as the essential oils of oregano, thyme, lemongrass, lemons, oranges, anise, cloves, aniseed, cinnamon, geraniums, roses, mint, peppermint, lavender, citronella, eucalyptus, sandalwood, cedar, rosmarin, pine, vervain fleagrass, and ratanhiae, or the aromatic compounds that comprise the essential oils, including methylglyoxal, carvacrol, eugenol, linalool, thymol, p-cymene, myrcene, borneol, camphor, caryophillin, cinnamaldehyde, geraniol, nerol, citronellol, and menthol. In further embodiments, the environmental conditioning composition contains about 0.001% by weight to about 1% by weight of the fragrant compound.

In other embodiments, a plurality of environmental conditioning compositions consisting essentially of water are dispersed during the course of the method. In some embodiments, an environmental conditioning composition consisting essentially of water is dispersed into the volumetric space prior to dispersing the first aqueous composition into the volumetric space, and an environmental conditioning composition consisting essentially of water is dispersed into the volumetric space after the peracid has been formed in situ within the reaction layer on the surface. In another embodiment, an environmental conditioning composition consisting essentially of water is dispersed into the volumetric space prior to dispersing the first aqueous composition into the volumetric space, and an environmental conditioning composition consisting essentially of water is dispersed into the volumetric space after the first aqueous composition layer is formed upon the surface and prior to dispersing the second aqueous composition into the volumetric space. In another embodiment, an environmental conditioning composition consisting essentially of water is dispersed into the volumetric space after the first aqueous composition layer is formed upon the surface and prior to dispersing the second aqueous composition into the volumetric space, and an environmental conditioning composition consisting essentially of water is dispersed into the volumetric space after the peracid has been formed in situ within the reaction layer on the surface. In another embodiment, an environmental conditioning composition consisting essentially of water is dispersed into the volumetric space prior to dispersing the first aqueous composition into the volumetric space, an environmental conditioning composition consisting essentially of water is dispersed into the volumetric space after the first aqueous composition layer is formed upon the surface and prior to dispersing the second aqueous composition into the volumetric space, and an environmental conditioning composition consisting essentially of water is dispersed into the volumetric space after the peracid has been formed in situ within the reaction layer on the surface

When dispersed as microdroplets, the effective diameter of the multiplicity of microdroplets of any of the supplemental aqueous compositions can be controlled similarly to the first aqueous composition or the second aqueous composition. In some embodiments, the effective diameter of a preponderance of the microdroplets of a supplemental aqueous composition is at least about 1 micron, including at least about 10 microns, 20 microns, 30 microns, 40 microns, 50 microns, or about 100 microns. In other embodiments, the effective diameter of a preponderance of microdroplets of a supplemental aqueous composition is between about 20 microns and about 30 microns. In still other embodiments, a preponderance of the multiplicity of microdroplets have an effective diameter of less than or equal to about 1 micron, including less than or equal to about 10 microns, 20 microns, 30 microns, 40 microns, 50 microns, or about 100 microns. Useful ranges for the effective diameter of the multiplicity of microdroplets of any of the supplemental aqueous compositions can be selected from any value between and inclusive of about 1 micron to about 100 microns. Non-limiting examples of such ranges can include from about 1 micron to about 100 microns, from about 10 microns to about 100 microns, from about 20 microns to about 100 microns, from about 30 microns to about 100 microns, from about 40 microns to about 100 microns, from about 50 microns to about 100 microns, or from about 20 microns to about 30 microns.

In some embodiments, multiple supplemental aqueous compositions can be dispensed within the same disinfection method. Non-limiting examples include methods that further comprise dispensing an environmental conditioning composition and a peracid scavenging composition; a pesticide composition and a peracid scavenging composition; an environmental conditioning composition and a pesticide composition; or an environmental conditioning composition, a pesticide composition, and a peracid scavenging composition. In further embodiments, the methods of the present invention further comprise dispensing multiple environmental conditioning compositions and either or both of a pesticide composition and a peracid scavenging composition.

As a non-limiting example, a method to disinfect a surface in need of disinfecting within a volumetric space can comprise the steps of: a) dispersing into the volumetric space an environmental conditioning composition consisting essentially of water; b) allowing a time sufficient time sufficient for the environmental conditioning composition to distribute throughout the volumetric space and cause the volumetric space to have a relative humidity of at least about 50 percent, including at least about 60, 70, 80, 90, or 95 percent, up to about 99 percent; c) dispersing into the volumetric space a multiplicity of microdroplets of a first aqueous composition comprising a first peracid reactant compound that is either a peroxide compound or an organic acid compound capable of reacting with a peroxide compound to form a peracid; d) allowing a time sufficient for the multiplicity of microdroplets of the first aqueous composition to distribute throughout the volumetric space and to deposit and coalesce into a first aqueous composition layer upon the surface; e) dispersing into the volumetric space a multiplicity of microdroplets of a second aqueous composition comprising a second peracid reactant compound that is the other of the first peracid reactant compound; f) allowing a second time sufficient for the multiplicity of microdroplets of the second aqueous composition to deposit onto the coalesced first aqueous composition layer to form a reaction layer upon the surface, thereby forming a peracid in situ within the reaction layer and disinfecting the surface; g) dispersing into the volumetric space a peracid scavenging composition comprising a metal halide compound; and h) allowing a time sufficient for the peracid scavenging composition to distribute throughout the volumetric space and to deposit onto the disinfected surface. In further embodiments, the method further comprises the steps of i) dispersing into the volumetric space an environmental conditioning composition consisting essentially of water; and j) allowing a time sufficient for the environmental conditioning composition to distribute throughout the volumetric space and to deposit onto the disinfected surface. In even further embodiments, the environmental conditioning composition in step i) further consists essentially of a fragrant compound.

In another non-limiting example, a method to disinfect a surface in need of disinfecting within a volumetric space can comprise the steps of: a) dispersing into the volumetric space an environmental conditioning composition consisting essentially of water; b) allowing a time sufficient time sufficient for the environmental conditioning composition to distribute throughout the volumetric space and cause the volumetric space to have a relative humidity of at least about 50 percent, including at least about 60, 70, 80, 90, or 95 percent, up to about 99 percent; c) dispersing into the volumetric space a pesticide composition; d) allowing a time sufficient for the pesticide composition to distribute throughout the volumetric space and to deposit onto the surface; e) dispersing into the volumetric space a multiplicity of microdroplets of a first aqueous composition comprising a first peracid reactant compound that is either a peroxide compound or an organic acid compound capable of reacting with a peroxide compound to form a peracid; f) allowing a time sufficient for the multiplicity of microdroplets of the first aqueous composition to distribute throughout the volumetric space and to deposit and coalesce into a first aqueous composition layer upon the surface; g) dispersing into the volumetric space a multiplicity of microdroplets of a second aqueous composition comprising a second peracid reactant compound that is the other of the first peracid reactant compound; h) allowing a second time sufficient for the multiplicity of microdroplets of the second aqueous composition to deposit onto the coalesced first aqueous composition layer to form a reaction layer upon the surface, thereby forming a peracid in situ within the reaction layer and disinfecting the surface; i) dispersing into the volumetric space a peracid scavenging composition comprising a metal halide compound; and j) allowing a time sufficient for the peracid scavenging composition to distribute throughout the volumetric space and to deposit onto the disinfected surface. In further embodiments, the method further comprises the steps of k) dispersing into the volumetric space an environmental conditioning composition consisting essentially of water; and l) allowing a time sufficient for the environmental conditioning composition to distribute throughout the volumetric space and to deposit onto the disinfected surface. In even further embodiments, the environmental conditioning composition dispersed into the volumetric space in step k) further consists essentially of a fragrant compound. In other further embodiments, the pesticide composition dispersed into the volumetric space in step c) comprises an insecticide, particularly an insecticide configured to kill bed bugs or termites.

As a consequence of utilizing one or more of the supplemental aqueous compositions, the present invention also provides safer and potentially more effective methods for disinfecting surfaces using already-formed peracids, especially in disinfecting applications in which the already-formed peracid is dispersed as a spray, fog, or vapor. As described above, problems associated with commercial peracid compositions used to disinfect surfaces typically comprise at least about 0.01% by weight peracid, including at least about 0.05%, 0.1%, 0.25%, 0.5%, 0.75%, 1%, 5%, 10%, 20%, or 30%, up to about 40% peracid by weight (see Centers for Disease Control “Guideline for Disinfection and Sterilization in Healthcare Facilities (2008)” viewed at http://www.cdc.gov/infectioncontrol/guidelines/disinfection/disinfection-methods/chemical.html, page last updated Sep. 18, 2016).

In some embodiments, the method for disinfecting a surface in need of disinfecting within a volumetric space using a pre-formed peracid comprises the steps of: a) dispersing into the volumetric space a multiplicity of microdroplets of a first aqueous composition comprising a peracid; and b) allowing a time sufficient for the first aqueous composition to distribute throughout the volumetric space and to deposit onto the surface, thereby disinfecting the surface; wherein the method further includes the step of dispersing into the volumetric space a multiplicity of microdroplets of one or more supplemental aqueous compositions selected from the group consisting of a peracid scavenging composition, a pesticide composition, and an environmental conditioning composition, and allowing a time sufficient for each dispersed supplemental aqueous composition to distribute throughout the volumetric space and to deposit onto the surface. In further embodiments, the peracid is peroxyacetic acid.

Particularly, utilizing a peracid scavenging composition after dispersing a peracid into the volumetric space and/or onto a surface can enhance deposition of any excess or lingering peracid from the volumetric space after being dispersed, or by removing the peracid from the surface after the surface has been disinfected. Similar to other methods of the present invention in which the peracid is formed in situ on the surface to be disinfected, the peracid scavenging composition can comprise a metal halide compound, particularly a metal halide compound selected from the group consisting of potassium iodide, potassium chloride, and sodium chloride, and more particularly potassium iodide. Because the peracid is in a pre-formed composition rather than being formed on the surface to be disinfected, in some embodiments it can be desirable or advantageous to disperse a stoichiometric amount of the metal halide compound dispersed into the volumetric space is equal or greater than the amount of the peracid dispersed into the volumetric space, to ensure that substantially all of the dispersed peracid is scavenged from the volumetric space. In further embodiments, the stoichiometric amount of the metal halide dispersed into the volumetric space is at least 2 times greater than the amount of the peracid dispersed into the volumetric space, including at least 3, 4, 5, 10, 25, 50, or 100 times greater than the amount of peracid dispersed into the volumetric space. When potassium iodide is included in the peracid scavenging composition, the peracid scavenging composition can comprise at least about 0.000001 moles per liter potassium iodide, including at least about 0.00001, 0.0001, 0.001, 0.01, 0.1, or about 1 mole per liter potassium iodide, up to about 6 moles per liter potassium iodide.

Similarly, an environmental conditioning composition consisting essentially of water can be dispersed either prior to or after dispersing the first aqueous composition comprising the pre-formed peracid. Particularly, dispersing the environmental conditioning composition after dispersing the first aqueous composition can have the effect of diluting, reducing, or removing lingering or excess peracid within the volumetric space after surfaces within the volumetric space are disinfected. Additionally, the environmental conditioning composition can further consist essentially of a fragrant compound, particularly a fragrant compound selected from the group consisting of methylglyoxal, carvacrol, eugenol, linalool, thymol, p-cymene, myrcene, borneol, camphor, caryophillin, cinnamaldehyde, geraniol, nerol, citronellol, and menthol, including combinations thereof.

On the other hand, when the environmental conditioning composition is dispersed into the volumetric space prior to dispersing the first aqueous composition into the volumetric space, the method can further include the step of allowing a time sufficient for the environmental conditioning composition to distribute throughout the volumetric space and cause the volumetric space to have a relative humidity of at least about 50 percent, including at least about 60, 70, 80, 90, or 95 percent, up to about 99 percent, in order to enhance the coverage and deposition of the first aqueous composition onto all of the desired surfaces within the volumetric space.

As with embodiments in which the peracid is formed in situ on the surface to be disinfected, any combination of supplemental aqueous compositions can be dispersed sequentially along with the first aqueous composition comprising the pre-formed peracid. In one non-limiting example, an environmental conditioning composition consisting essentially of water can be dispersed into the volumetric space prior to dispersing the first aqueous composition, and a peracid scavenging composition can be dispersed into the volumetric space after the surface has been disinfected. In another non-limiting example, a pesticide composition can be dispersed into the volumetric space either before or after dispersing the first aqueous composition. In yet another non-limiting example, a pesticide composition can be dispersed into the volumetric space prior to dispersing the first aqueous composition, a peracid scavenging composition can be dispersed into the volumetric space after the surface has been disinfected, and an environmental conditioning composition consisting essentially of water and a fragrant compound can be dispersed into the volumetric space after substantially all of the peracid has been removed from the volumetric space. Those skilled in the art would appreciate that several other combinations exist in which one or more supplemental aqueous compositions are dispersed sequentially in conjunction with dispersing the first aqueous composition comprising a pre-formed peracid.

In other embodiments of the invention, particularly embodiments in which the aqueous compositions are dispersed as a liquid stream, a multiplicity of droplets, or as a vapor, the time sufficient for any of the first aqueous composition, the second aqueous composition, or any of the supplemental aqueous compositions to distribute throughout the volumetric space, deposit onto the surface, and/or form an aqueous composition layer upon the surface can be defined to be a specific unit of time. As a non-limiting example, mechanized or automated spray, fogging, or delivery systems, as described below, can include a programming to require a delay between dispersing an aqueous composition and dispersing a subsequent aqueous composition. In some embodiments, the time sufficient for an aqueous composition to distribute throughout a volumetric space and/or deposit onto a surface is at least about 1 second, including about 10 seconds, 30 seconds, 1 minute, 2 minutes, 3 minutes, 4 minutes, 5 minutes, or 10 minutes, up to at least about 15 minutes.

In another non-limiting example, a method of disinfecting a surface in need of disinfecting within a volumetric space can comprise the steps of: a) dispensing onto the surface a quantity of a first aqueous composition comprising a first peracid reactant compound that is either a peroxide compound or an organic acid compound capable of reacting with a peroxide compound to form a peracid; b) allowing a time sufficient for the first aqueous composition to deposit onto the surface and coalesce into a first aqueous composition layer upon the surface, wherein the time sufficient is at least about 1 second, including about 10 seconds, 30 seconds, 1 minute, 2 minutes, 3 minutes, 4 minutes, 5 minutes, or 10 minutes, up to at least about 15 minutes; c) dispensing onto the surface a quantity of a second aqueous composition comprising a second peracid reactant compound that is the other of the first peracid reactant compound; and d) allowing a second time sufficient for the second aqueous composition to deposit onto the surface and combine with the coalesced first aqueous composition layer to form a reaction layer upon the surface, wherein the second time sufficient is at least about 1 second, including about 10 seconds, 30 seconds, 1 minute, 2 minutes, 3 minutes, 4 minutes, 5 minutes, or 10 minutes, up to at least about 15 minutes, thereby forming a peracid in situ within the reaction layer and disinfecting the surface. In even further embodiments, the method further comprises the steps of dispersing into the volumetric space one or more supplemental aqueous compositions and allowing a time sufficient for each dispersed supplemental aqueous composition to distribute throughout the volumetric space and to deposit onto the surface, wherein the time sufficient is at least about 1 second, including about 10 seconds, 30 seconds, 1 minute, 2 minutes, 3 minutes, 4 minutes, 5 minutes, or 10 minutes, up to at least about 15 minutes.

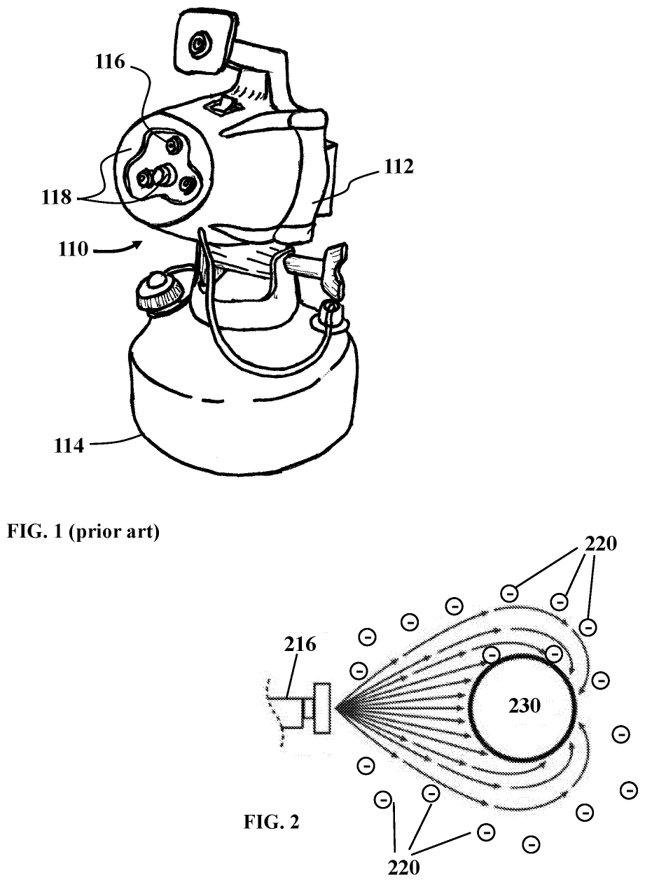

In another embodiment of the invention, the multiplicity of microdroplets of any of the aqueous compositions described above can be electrostatically charged. An example of electrostatic spraying is described in U.S. Pat. No. 6,692,694, the disclosure of which is incorporated by reference in its entirety. FIG. 1 illustrates an example of a commercial electrostatic spray device 110 according to the prior art. Electrostatic spray device 110 includes a housing 112; a container 114 associated with the housing 112 for storing a liquid; multiple nozzles 116 in liquid communication with the container 114 for dispensing aerosolized microdroplets of the liquid; and a high voltage charging system 118 capable of imparting an electrostatic charge on the microdroplets after they are dispersed. Those skilled in the art would appreciate that any electrostatic spray device can be utilized to disperse electrostatically-charged microdroplets, including devices that spray microdroplets having only a positive charge, devices that spray microdroplets having only a negative charge, and devices that are adjustable to selectively spray microdroplets having any desired charge. In some embodiments, an electrostatic spray device that is adjustable to selectively spray microdroplets having either a positive, negative, or neutral charge can be utilized.

There are several advantages that can be exploited by dispersing the microdroplets with an electrostatic charge, including but not limited to: a more effective and targeted dispersal onto surfaces to be disinfected, application onto non-line-of-sight vertical and under-side surfaces, and enhanced activation of the peracid reactant compounds prior to the formation of the peracid on the surface. Without being limited by theory, it is believed that applying an electrostatic charge leads to a more effective dispersal of the aqueous composition because the multiplicity of like-charged microdroplets repels each other according to Coulomb's law. As shown in FIG. 2, negatively charged particles 220 dispensed from the nozzle of an electrostatic spray device 216 will deposit onto all faces of a positive or neutrally-charged surface 230. Microdroplets will additionally distribute evenly across an area or volumetric space and deposit on to a diversity of surfaces, including the back surfaces and underside surfaces, of an object in an effort to maximize the distance from microdroplet to microdroplet.

Because of the volume of the aqueous composition dispersed in the volumetric space, the like-charged particles can spontaneously coalesce into a layer on the surface. In some embodiments, the first aqueous composition is electrostatically charged to provide a uniformly distributed layer of first aqueous composition layer on the surfaces to be disinfected, after which the second aqueous composition is dispersed into the volumetric space. In other embodiments, Coulomb's law can be further exploited by electrostatically charging the multiplicity of microdroplets of the second aqueous composition with the opposite polarity as the multiplicity of microdroplets of the first aqueous composition, creating an attraction between the first aqueous composition layer and the multiplicity of microdroplets of the second aqueous composition, and ensuring that the peracid reactant compounds come into contact with each other to form a reaction layer on the surface to be disinfected.

Additionally, the electrostatic charge placed on an aqueous composition can be selected to enhance the reactivity of the peracid reactant compounds. In some embodiments, the aqueous composition that includes the peroxide compound can be electrosprayed with a negative charge, while the aqueous composition including the organic acid compound can be electrosprayed with a positive charge. In other embodiments, the aqueous composition that includes the peroxide compound can be electrosprayed with a positive charge and the aqueous composition that includes the organic acid compound can be sprayed with a negative charge. Ultimately, any combination of electrostatic charge (positive, negative, or neutral) can be applied to any aqueous composition, independent of the identity of the components present in either aqueous composition.

In addition to augmenting the deposition of the aqueous compositions on the surfaces to be disinfected and enhancing the peracid-forming reaction, utilizing electrospray technology brings additional supplemental benefits to the methods described herein. While the attraction that the electrostatically-charged microdroplets have for surfaces is beneficial for facilitating the reaction on the surfaces to be disinfected, it also provides an additional safety measure if anyone enters the volumetric space during disinfection. Without being limited by a particular theory, it is believed that smaller microdroplets that would otherwise penetrate into someone's deep lung would instead be attracted to the surfaces of that person's nasal cavity or mouth, where the effects of the microdroplets, if any, can be easily neutralized. Additionally, the repulsion experienced by identically-charged particles can cause microdroplets to remain in the air for a longer period of time without being forced to the ground by gravity. Thus, larger microdroplet sizes can be used and disinfection of surfaces within larger volumetric spaces can be facilitated.

In some embodiments, surfaces within the volumetric space can also be galvanically grounded prior to dispersing the first aqueous composition by electrostatic spray. In further embodiments, surfaces can be earth-grounded. Because an electric attraction is created between the grounded surfaces and the charged microdroplets in the volumetric space, the microdroplets can become attracted preferentially, or only, to the grounded surfaces. As a non-limiting example, high-traffic or highly-contaminated surfaces in a hospital room such as door handles, faucets, and hospital bedrails and bars, can be targeted by grounding them prior to disinfecting, facilitating a faster turnover of the room between patients. In other embodiments, surfaces that are already grounded within an area or volumetric space can be isolated from the ground prior to dispersing an electrostatically-charged first aqueous composition, in order to provide a better blanket coverage of all surfaces within the volumetric space. In further embodiments, electrostatically spraying selected grounded surfaces with the first aqueous composition can be utilized in combination with dispersing a second aqueous composition with no electrostatic charge in order to provide general surface coverage throughout the volumetric space.

In some embodiments, an electrostatic charge may be applied either prior to the aerosolization of the aqueous composition or after the composition has been dispersed. Distribution of the multiplicity of electrostatically-charged microdroplets can be controlled by adjusting the magnitude of the voltage applied to the nozzle on the electrostatic sprayer, nozzle size or type, and the flow rate of the aqueous composition through the nozzle.

In some embodiments, particularly when the surface to be disinfected is difficult to contact, such as inside an air duct or in a confined space, or when there are several surfaces to be disinfected in a very large volumetric space, vaporizing the aqueous compositions in the ambient air or introducing them into a hot gaseous stream can be effective. Sterilization using these methods has been described in U.S. Pat. Nos. 8,696,986 and 9,050,384, the disclosures of which are incorporated by reference in their entireties. Similar to the other patent references described above, the methods described in U.S. Pat. Nos. 8,696,986 and 9,050,384 require that the peracid be formed and then dispensed into a volumetric space. In contrast, peracid reactant compounds according to methods of the present invention can be dispersed in separate application steps, thereby forming the peracid in situ only on the surfaces to be disinfected.

As a non-limiting example, a surface in need of disinfecting within an volumetric space containing ambient air may be disinfected using a method comprising the steps of: a) heating a first aqueous composition comprising a peroxide compound to produce a vapor comprising the peroxide compound in the ambient air; b) allowing a first time sufficient for the vapor comprising the peroxide compound to distribute throughout the volumetric space, and to cool, condense and deposit into a liquid layer upon the surface, the liquid layer comprising the peroxide compound; c) heating a second aqueous composition comprising an organic acid compound to produce a vapor comprising the organic acid compound; and d) allowing a second time sufficient for the vapor comprising the organic acid compound to distribute throughout the volumetric space, and to cool, condense and deposit the organic acid compound onto the liquid layer comprising the peroxide compound to form a reaction layer, thereby forming a peracid in situ on the reaction layer and disinfecting the surface.

In some embodiments, in order to form a vapor, an aqueous composition can be pressure fed into an atomizing device wherein the composition is mechanically introduced as a high-pressure mist into ambient temperature atmospheric air, forming a mist or spray. The mist or spray is then heated and vaporized by repeatedly passing the mist or spray in close proximity to one or more heating elements integral to the atomizing device. As the aqueous composition repeatedly circulates, it further energizes into a superheated vapor at any user selectable temperature, for example, greater than or equal to about 250° C. Alternatively, the aqueous composition can be heated at a temperature sufficient to vaporize a mass of the aqueous composition in less than about 30 minutes, including less than about 25, 20, 15, 10, or about 5 minutes. In a further embodiment, the aqueous composition can be heated at a temperature sufficient to vaporize the mass of the aqueous composition in about two minutes.

After exiting the atomizing device, the superheated vapor cools and condenses into a multiplicity of microdroplets as it disperses and settles through the air. In use, the atomizing device can be located a sufficient distance from the surface to be disinfected such that the temperature of the condensed microdroplets as they deposit on the surface is less than or equal to about 55° C. In some embodiments, the condensed microdroplets are applied at a temperature approximating the ambient temperature in the storage facility, optimally ranging from about 10° C. to about 25° C. By allowing the vapor to condense into microdroplets and cool to an approximately ambient temperature, the user can safely apply the vapor to both inert solid surfaces and the non-inert surfaces of agricultural products. In embodiments in which the entire method is applied over periods of time ranging from 40 minutes to 8 hours, substantially all surfaces can be disinfected within the volumetric space, killing virtually all bacteria, bacterial spores, fungi, protozoa, algae, and viruses on both stored agricultural products and on the surfaces of the storage facilities in which the agricultural products are stored.

Similar to other embodiments of this invention described above in which liquid microdroplets of the aqueous compositions are dispersed into the air, disinfection methods according to the present invention that involve vaporization also show a diminished effectiveness in dry environments. Thus, in some embodiments, the vaporization methods may further include the step of pre-treating the volumetric space by dispersing an environmental conditioning composition consisting essentially of water to increase the humidity of the area.

In another embodiment of the invention, aqueous compositions can be vaporized by introducing them into a hot gaseous stream prior to their dispersion into the volumetric space. In some embodiments, the heated gas stream is sterile air, although other gases such as nitrogen, CO₂, or inert noble gas carriers can also be used. The gas stream can be heated to any user-controlled temperature above about 250° C. An aqueous composition can be introduced into the air stream by any means well known to one of skill in the art. In preferred embodiments, the aqueous composition is dispersed directly into the stream. Similar to the embodiments described above, once the vapor containing the aqueous composition is dispersed into the volumetric space, the time sufficient for the vapor to cool, condense into a multiplicity of microdroplets, and deposit into a liquid layer upon a surface will vary depend on factors including but not limited to the identity and concentration of the components in the aqueous composition and the nature of the material of the surface to be disinfected.

In a further embodiment of the invention, any of the above-described methods may further include the step of illuminating the surface to be disinfected with a wavelength consisting essentially of ultraviolet (UV) light. UV light is known to kill pathogens in the air, on surfaces, and in liquids. Methods employing UV light to kill pathogens are described in U.S. Pat. Nos. 6,692,694 and 8,110,538, the disclosures of which are incorporated by reference in their entireties. In addition to having its own biocidal activity, UV light can activate peroxide compounds to make them even more reactive in reactions with organic acid compounds to form peracids. For example, hydrogen peroxide can be activated when it is bombarded by intense UV light to form two hydroxyl radicals. In preferred embodiments, once an aqueous composition including a peroxide compound has deposited and coalesced upon a surface to be disinfected, the surface is then illuminated with a wavelength consisting essentially of UV light. Alternatively, the aqueous composition containing the peroxide compound may be illuminated with a wavelength consisting essentially of UV light as it is dispersed. UV light may be generated using any means well known to one of skill in the art.

In some embodiments of the invention, the disinfectant methods described above for generating peracids on surfaces to be disinfected can be used for a variety of user-identified biocidal purposes, including antimicrobial, bleaching, or sanitizing applications. In other aspects, the generated peracids may be used to kill one or more of the food-borne pathogenic bacteria associated with a food product, including, but not limited to Salmonella typhimurium, Campylobacter jejuni, Listeria monocytogenes, and Escherichia coli 0157:H7, yeast, and mold.

In some embodiments, the peracids generated according to the methods and system of the present invention are effective for killing one or more of the pathogenic bacteria associated with health care surfaces and instruments including but not limited to, Salmonella typhimurium, Staphylococcus aureus, Salmonella choleraesurus, Pseudomonas aeruginosa, Escherichia coli, Mycobacteria, yeast, and mold.

Furthermore, the peracids generated according to the methods and system of the present invention are effective against a wide variety of microorganisms, such as Gram-positive organisms (Listeria monocytogenes or Staphylococcus aureus), Gram-negative organisms (Escherichia coli or Pseudomonas aeruginosa), catalase-positive organisms (Micrococcus luteus or Staphylococcus epidermidis), or sporulent organisms (Bacillus subtilis).

In some embodiments of the invention, the methods can be practiced using solely food-grade components. For example, though not required, the disinfectant methods in this invention can be practiced substantially free of ingredients commonly present in many commercially available surface cleaners. Examples of non-food grade components that can be omitted include, but are not limited to, aldehydes such as glutaraldehyde, chlorine- and bromine containing components, iodophore-containing components, phenolic-containing components, quaternary ammonium-containing components, and others. Furthermore, because peracids are formed in situ on the surface to be disinfected, heavy transition metals, surfactants, or other stabilizing compounds that could be used to prevent hydrolysis of the peracid prior to disinfecting the target surface are also not necessary and can be omitted from aqueous compositions coming into contact with food preparation surfaces or food itself.

Accordingly, the methods to produce peracids directly on surfaces to be disinfected can be employed on foods and plant species to reduce surface microbial populations, or at manufacturing, processing, or refrigerated and non-refrigerated transportation sites handling such foods and plant species. For example, the compositions can be used on food transport lines (e.g., as belt sprays); boot and hand wash dip-pans; food storage facilities; shipping containers; railcars; anti-spoilage air circulation systems; refrigeration and cooler equipment; beverage chillers and warmers; blanchers; cutting boards; third-sink areas; and meat chillers or scalding devices.

### Sequential Application and Delivery Systems

In addition to the chemical methods described above for disinfecting one or more surfaces within a volumetric space, the present invention also provides several sequential application and delivery systems that are configured for carrying out those methods. The sequential application and delivery systems can sequentially dispense two or more liquid compositions onto surfaces within the volumetric space so the two or more liquid compositions can interact chemically or physically upon the surface.

In some embodiments, the sequential application and delivery system can dispense a first liquid composition into the volumetric space, and after a time sufficient for the first liquid composition to distribute throughout the volumetric space and deposit and coalesce into a layer upon one or more surfaces within the volumetric space, the system can dispense a second liquid composition. Once the second liquid composition deposits onto the coalesced layer of the first liquid composition on a particular surface, the two liquid compositions can interact with each other in situ on the surface. In further embodiments, the interaction between the first liquid composition and the second liquid composition comprises a chemical reaction, wherein a chemical reaction product is formed in situ within a reaction layer formed upon the surfaces within the volumetric space. In other further embodiments, the interaction between the first liquid composition and the second liquid composition comprises a physical interaction in which the physical properties of the first liquid composition and the second liquid composition are combined and/or enhanced.

In some embodiments, the liquid compositions are aqueous compositions. In other embodiments, the liquid compositions are non-aqueous compositions, including but not limited to oil-based compositions, organic compounds or compositions, and other volatile compounds or compositions that are substantially free of water. Instances in which the sequential application and delivery systems can be used in addition to the disinfection and sterilization methods described above, include but are not limited to, painting, staining, chemical treatments, application of anti-corrosive coatings, personal health and beauty treatments, and lawn care fertilization and maintenance.

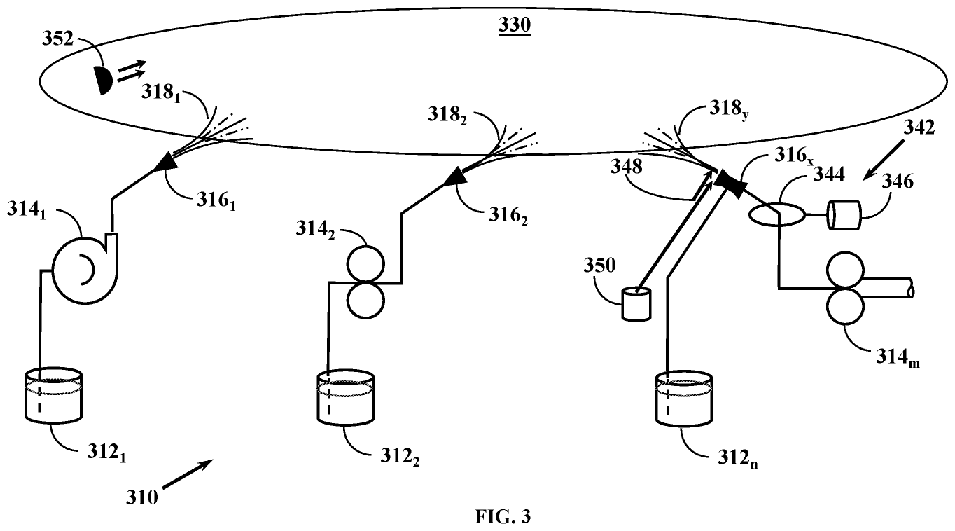

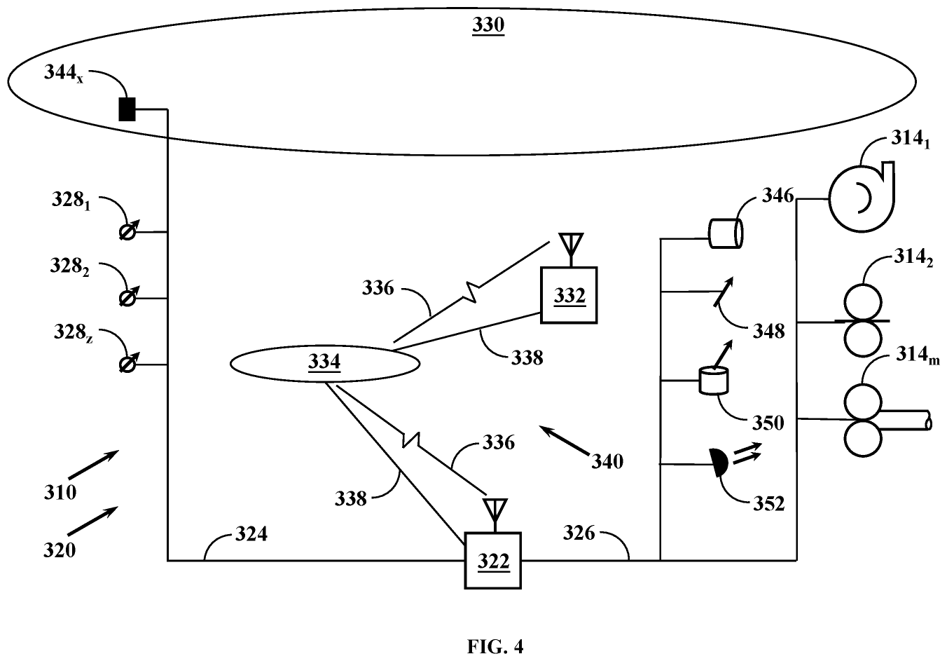

In some embodiments and as illustrated in FIG. 3, the sequential application and delivery system 310 comprises a plurality of aqueous composition containers 312₁₋ₙ, each configured for housing or containing an aqueous composition, a plurality of associated dedicated pumps 314₁₋ₘ, each in fluid communication respectively with one of the containers 312₁₋ₙ therewith, and one or more aqueous composition delivery nozzles 316₁₋ₓ, each in fluid communication with a respective pump 314₁₋ₘ and configured to deliver aqueous compositions as indicated at reference numerals 318_{1-y} into a volumetric space 330. In various embodiments, the plurality of associated dedicated pumps 314₁₋ₘ. can, for example, be one of several types including, but not necessarily limited to, a centrifugal pump 314₁, a metering pump 3142, and a venturi pump 314ₘ. As illustrated in FIG. 4, the sequential application and delivery system 310 further includes a data acquisition and control system 320 generally comprising a central processing unit or controller 322, a data acquisition bus 324, and a control bus 326. More specifically, the controller 322 is electrically coupled to the aqueous composition containers 312₁₋ₙ through the data acquisition bus 324 and is configured to ascertain, e.g., read, a respective means 328_{1-z} for detecting the aqueous compositions levels in each of the aqueous composition containers 312₁₋ₙ. Such means include, but are not necessarily limited to, float, capacitance, conductivity, ultrasonic, radar level, and optical sensors. The controller 322 is also electrically coupled to respective drives, e.g., motors, for the pumps 314₁₋ₘ through the control bus 326 and is configured to power the pumps 314₁₋ₘ to dispense aqueous compositions from the aqueous composition containers 312₁₋ₙ to and through the aqueous composition delivery nozzles 316₁₋ₓ, into the volumetric space 330.

In some embodiments, the pumps 314₁₋ₘ can be replaced with a motor and a piston member contained within each aqueous composition container 312₁₋ₙ to force an aqueous composition out of each container 312₁₋ₙ rather than having the pumps 314₁₋ₘ draw or suck the aqueous composition out of the containers 312₁₋ₙ without departing from the spirit of the present invention.

In use, the controller 322 is programmed to dispense the first aqueous composition 318₁ into the volumetric space 330, based on a pre-programmed quantity of the aqueous composition, or a pre-programmed first rate of dispensing the aqueous composition for a period of time t₁. After the dispensing of the first aqueous composition has ceased and after a time sufficient for the first aqueous composition 318₁ to distribute throughout the volumetric space 330 and deposit and coalesce into a first aqueous composition layer upon surfaces within the volumetric space 330, the controller 322 is programmed to dispense a second aqueous composition 318₂, again, based on the quantity and/or rate of dispensing the aqueous composition for a period of time t₂. The controller 322 can also be programmed to sequentially dispense supplemental aqueous compositions into the volumetric space 330 at various intervals.

Further, as illustrated in FIG. 4, the programming can be resident, contained within the controller 322, or distributed or resident elsewhere, such as in a remote controller or processor 332, across a network 334, for example, a local area network (LAN) or wireless local area network (WLAN). The network 334 can be wired 338 or wireless 336, or a combination of wired 338 and wireless 336. In some embodiments, hardware components containing the programming can provide for communicating with programming resident located outside of the volumetric space 330 to obtain the necessary information. It will be appreciated by one of ordinary skill in the art that the computational environment 340 in no way limits the present invention and that dedicated and application-based software can be used without departing from the spirit of the present invention.

In some embodiments, the sequential application and delivery system 310 can further comprise one or more sensors 344x in data communication with the data bus 324, to be located in or proximate or adjacent to the volumetric space 330 while the disinfecting method is being conducted, as shown in FIG. 4. In some embodiments, the sensor 344ₓ can be configured and used to detect one or more functions within the volumetric space 330 while the sequential application and delivery system 310 is being prepared, in use, or after all dispensing of aqueous compositions has been completed. Non-limiting examples of such functions include: detection of motion or presence of humans or mammals within the volumetric space 330; coordinate dimensions of the volumetric space 330; the presence and identification of the variety of objects and surfaces within the volumetric space 330, including the material or composition of those objects; and the temperature, pressure, or relative humidity within the volumetric space 330. Such means can comprise mechanical and/or electrical sensors, such as global positioning system (GPS) detectors, infrared sensors, accelerometers, and Doppler-based, thermal-based, camera-based, audio-based, or light-based mechanisms, particularly laser-based mechanisms.

In some embodiments, the sensor 344ₓ can be configured and used to ascertain the size of the volumetric space 330. Non-limiting examples of sensors capable of ascertaining the size of the volumetric space 330 include three-axis coordinate-system, Doppler distance measuring apparatuses. In other embodiments, information about the volumetric space 330, including room dimensions, can be pre-loaded into the controller 322 either through an interface on the apparatus itself or through an interface on an electrically connected remote controller or processor 332, such as a tablet, smartphone, or a laptop. In further embodiments, the remote controller or processor 332 can be connected physically, i.e., wired, or wirelessly by Wi-Fi™ or Bluetooth® technologies, using unrestricted frequency bands designated by the Federal Communications Commission.

In some embodiments, the sensor 344ₓ can be configured and used to measure the humidity or relative humidity within the volumetric space 330. In some embodiments, the sequential application and delivery system 310 can be configured to dispense an aqueous composition consisting essentially of water or other reactively inert components into the volumetric space 330 in response to the sensor 344ₓ detecting a relative humidity that is below a desired threshold. In further embodiments, the sequential application and delivery system 310 can be configured to cease dispensing the aqueous composition consisting essentially of water in response to raising the relative humidity to the desired threshold. In even further embodiments, the relative humidity threshold is at least about 50%, including at least about 60%, 70%, 80%, 90%, or 95%, up to about 99%. In embodiments in which the sequential application and delivery system 310 comprises a single nozzle 316₁, an aqueous composition consisting essentially of water or other reactively inert components can be dispersed immediately at the end of dispersing either or both of the first and second aqueous compositions to clear the aqueous composition and its components from the supply line and nozzle body.

In some embodiments, the controller 322 can utilize information determined or estimated by one or more sensors 344ₓ prior to dispensing, including the size of the volumetric space 330, the relative humidity within the volumetric space 330, and/or the desired effective uniform thickness of the coalesced layer, to determine the appropriate volume of the aqueous compositions to dispense in order to contact all of the intended surfaces with the desired amount of each aqueous composition. In use, calculations made or performed by the controller 322 based on pre-programmed data or information detected by the one or more sensors 344ₓ can specify a specific quantity, rate, and/or time to dispense a particular aqueous composition, and can implement a calculated or pre-programmed time delay between dispensing the first aqueous composition, the second aqueous composition, and any other aqueous compositions. Additionally, the controller 322 can be programmed to select from one or more optional pre-programmed protocols, including protocols in which a composition consisting essentially of water or other inert, non-reactive materials is dispersed prior to dispersing the first aqueous composition, after dispersing the first aqueous composition and before the second aqueous composition, or after dispersing the second aqueous composition.

In some embodiments, the nozzle 316x can be constructed, modified, or adapted to disperse the aqueous compositions as microdroplets. In use, the nozzle 316x can be directed by the controller 322 to disperse a preponderance of the multiplicity of microdroplets having an effective diameter of at least about 1 micron, including at least about 5, 10, 15, 20, 25, 30, 35, 40, 45, 50, 60, 70, 80, or 90 microns, up to about 100 microns, into the volumetric space 330.

In some embodiments, the sequential application and delivery system 310 can optionally further comprise an ionizing device 348, illustrated in FIG. 3 and FIG. 4, such as an ionizing needle or high voltage charging system, proximate to the nozzle 316ₓ, configured to electrostatically charge microdroplets of the aqueous composition dispensed by the nozzle 316ₓ. Those skilled in the art would appreciate that devices capable of dispersing electrostatically-charged microdroplets of an aqueous composition disperse microdroplets having a positive, negative, or neutral charge, including devices that spray microdroplets having only a positive charge, devices that spray microdroplets having only a negative charge, and devices that are adjustable manually or by the controller 322 to selectively spray microdroplets having any desired charge. Furthermore, the amount of voltage applied by the ionizing device 348 can be varied using the controller 322 electrically coupled thereto.

In some embodiments, the sequential application and delivery system 310 optionally further comprises a vaporizer 350 having an output proximate to a nozzle 316ₓ. The vaporizer 350 is electrically coupled and responsive to the controller 322 via the control bus 326. In use, the controller 322 energizes the vaporizer 350 causing the vaporizer 350 to emit a hot gaseous stream. In conjunction with the emission of the hot gaseous stream, the controller 322 also energizes an associated pump 314ₘ to dispense an aqueous composition as shown at 318_{y}. The hot gaseous stream contacts the aqueous composition at 318_{y} and vaporizes the aqueous composition at 318_{y} and disperses the aqueous composition into the volumetric space 330 as a vapor.

In use, aqueous compositions 318_{1-y} can be heated, separately, by the vaporizer 350, to a temperature of greater than about 250° C. Alternatively, the aqueous compositions 318_{1-y} can be heated, separately, to a temperature sufficient to vaporize the mass of the first aqueous composition and the second aqueous composition in a vaporizing time of less than about 30 minutes, including less than about 25, less than about 20, less than about 15, less than about 10, or less than about 5 minutes. In a further embodiment, the first aqueous composition and the second aqueous composition can be heated, separately, to a temperature sufficient to vaporize the mass of the first aqueous composition and the second aqueous composition in about 2 minutes.

In some embodiments, the sequential application and delivery system 310 can optionally further include a means for illuminating at least one of the dispensed aqueous compositions, the reaction layer, and/or surfaces within the volumetric space 330 with a wavelength consisting essentially of ultraviolet light, for example an ultraviolet light emitting diode 352 responsive to controller 322.

Those of ordinary skill in the art will appreciate that sequential application and delivery system 310 can be packaged and mobilized in a variety of ways for delivering aqueous compositions 318_{1-y} into a volumetric space 330. In some embodiments, sequential application and delivery system 310 can be mobilized and transported into a volumetric space 330 as a human-carried apparatus, such as a hand-carried dispensing unit or backpack. In other non-limiting examples, sequential application and delivery system 310 can also be configured as or integrated into a handcart, cart, or optically controlled and/or directed trolley that is mobilized by a living being or through mechanized drive means.

In some embodiments, the sequential application and delivery system 310 can be packaged such that the aqueous solution containers 312₁₋ₙ comprise a subassembly that is installed on-site into the sequential application and delivery system 310, for delivering aqueous compositions 318_{1-y} into a volumetric space 330.

In another embodiment, sequential application and delivery system 310 can also be carried by one or more robots or drones to direct dispersion of one or more aqueous compositions onto targeted surfaces within the volumetric space 330, particularly within volumetric spaces that are very large or irregularly shaped, or where spraying electrostatically-charged microdroplets of the aqueous compositions is impractical. Each robot or drone can be configured to autonomously navigate along the floor or airspace within the volumetric space 330, and includes a central processing unit, controller, or microcontroller that performs various roving or flight operations to facilitate the autonomous execution of one or more services or tasks. Autonomous operations can include, but are not limited to: determining and executing an optimal path throughout the volumetric space 330 while meeting certain objectives and flight constraints, such as energy requirements; obstacle recognition allowing drones to autonomously avoid obstacles such as walls, humans, buildings, trees, etc. along its path; trajectory generation (i.e., motion planning) to determine optimal control maneuvers in order to follow a path necessary to complete the requested service or task; task regulation to determine specific control strategies required to constrain the robot or drone within some tolerance or permissible floor- or airspace; task allocation and scheduling to determine the optimal distribution of each service request/task among a plurality of service requests/tasks within time and equipment constraints; and cooperative tactics to formulate an optimal sequence and spatial distribution of activities between other robots or drones to maximize the effectiveness of the sequential application and delivery system 310. Extensive discussion of the use of robots and drones, particularly with respect to disinfection methods and systems, is described in U.S. Pat. Nos. 9,447,448 and 9,481,460, and International Patent Publication Nos. WO 2011/139300 and WO 2016/165793, the disclosures of which are incorporated by reference in their entireties.

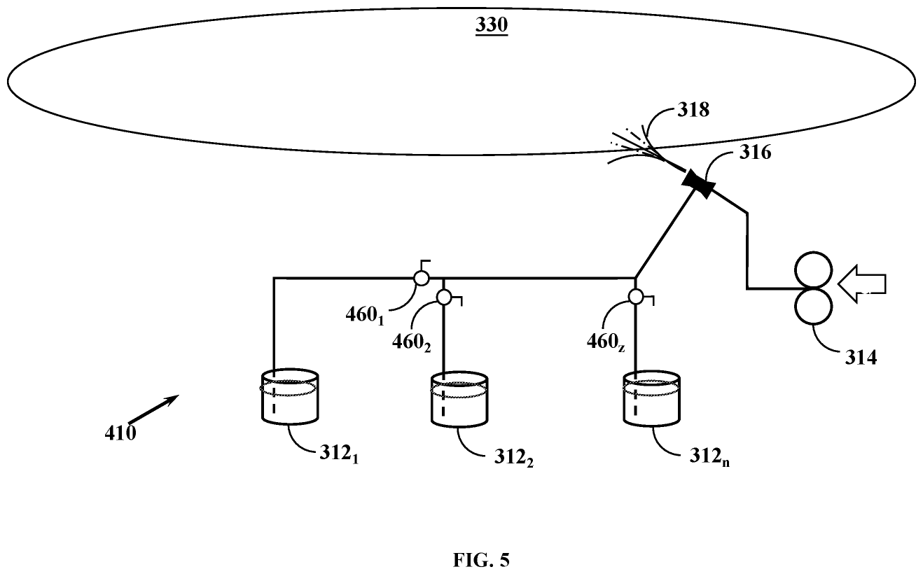

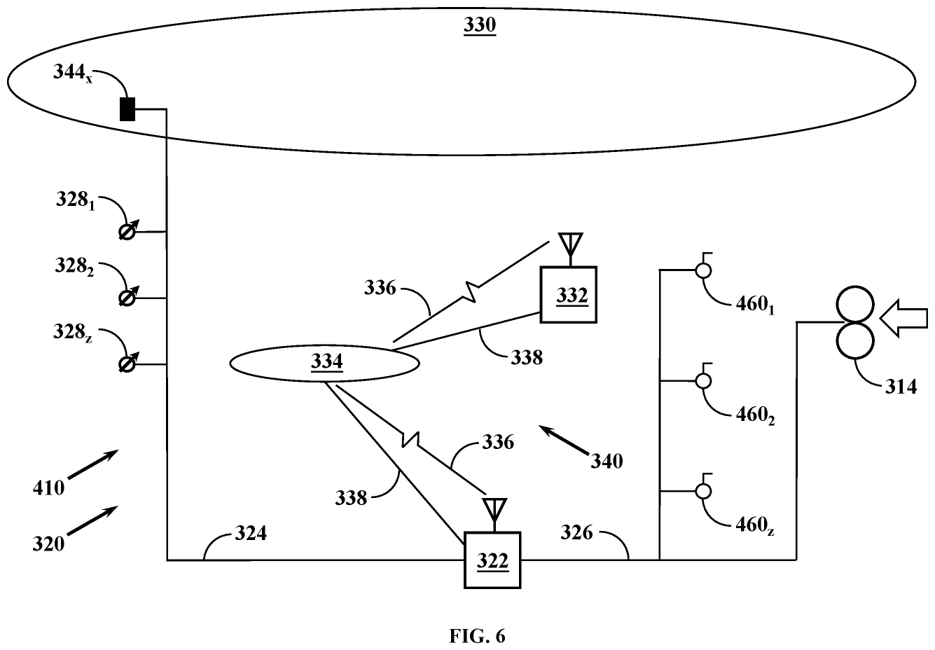

In other embodiments, and as illustrated by FIG. 5 and FIG. 6, the sequential application and delivery system 410 can include a single pump 314 and a plurality of controlled flow selection valves 360_{1-z} each respectively associated with aqueous composition containers 312₁₋ₙ. As shown, the controlled flow selection valves 360_{1-z} are electrically coupled to the controller 322 via the control bus 326.

In some embodiments, the controller 322 for the sequential application and delivery system 410 is configured to programmatically control flow selection valves 460_{1-z} to dispense aqueous compositions 318 into the volumetric space 330. As illustrated in FIG. 5, dispensed aqueous compositions 318 originate from a single nozzle 316. In some embodiments, the controller 322 can be programmed to selectively open and close flow selection valves 460_{1-z} to ensure that there is no unwanted mixing of the aqueous composition comprising the peroxide compound and the aqueous composition comprising the organic acid compound within the sequential application and delivery system 410 and before either composition reaches the surface(s) to be disinfected. In further embodiments, a supplemental aqueous composition can be circulated within the sequential application and delivery system 410 to neutralize and/or purge lingering any aqueous composition that remains within the system after the aqueous composition is dispersed into the volumetric space 330. In one non-limiting example, in a first step, a first aqueous composition is dispensed from aqueous composition container 3122 through an opened flow selection valve 4602, past a closed flow selection valve 460_{z}, and out of the single nozzle 316. In a second step, the controller closes flow selection valve 4602, opens flow selection valve 460₁, and circulates water housed in aqueous composition container 312₁ until it is dispensed from the nozzle 316, effectively removing all of the first aqueous composition from the sequential application and delivery system 410 before the second aqueous composition, housed in aqueous composition container 312ₙ, is dispersed into the volumetric space 330.

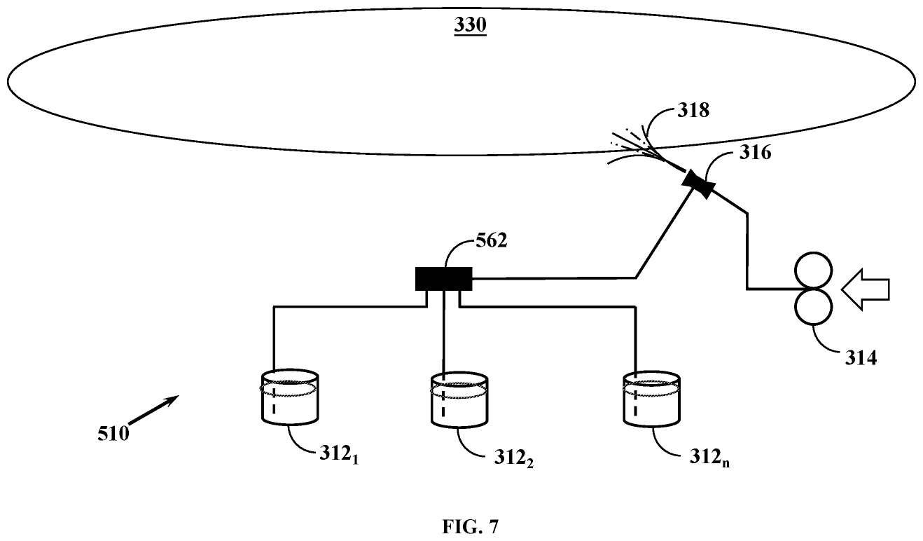

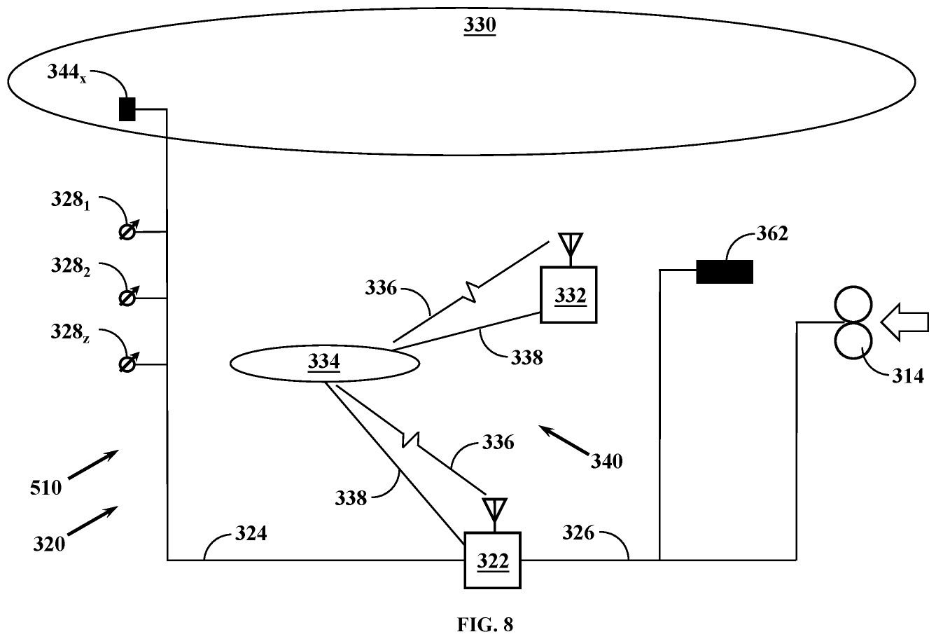

In some embodiments, and as illustrated by FIG. 7 and FIG. 8, the sequential application and delivery system 510 can include a single pump 314 and a controlled multi-way flow selection valve 562 associated with aqueous composition containers 312₁₋ₙ. As shown, the controlled multi-way flow selection valve 562 is electrically coupled to the controller 322 via the control bus 326.

In operation, and in some embodiments, the controller 322 is configured to programmatically control multi-way flow selection valve 562 to dispense aqueous compositions into the volumetric space 330. Similar to the sequential application and delivery system 410 above, the controller 322 within sequential application and delivery system 510 can be programmed to selectively control the flow through the multi-way flow selection valve 562 to ensure that there is no unwanted mixing of the aqueous composition comprising the peroxide compound and the aqueous composition comprising the organic acid compound before either composition reaches the surface(s) to be disinfected.

Additionally, the present invention provides sequential application and delivery systems configured to control the precise, automated execution of routines in which two or more liquid compositions are sequentially dispensed onto surfaces within a volumetric space, particularly routines in which the user is positioned outside of the volumetric space and possesses a device for communicating with one or more sprayers inside the volumetric space.

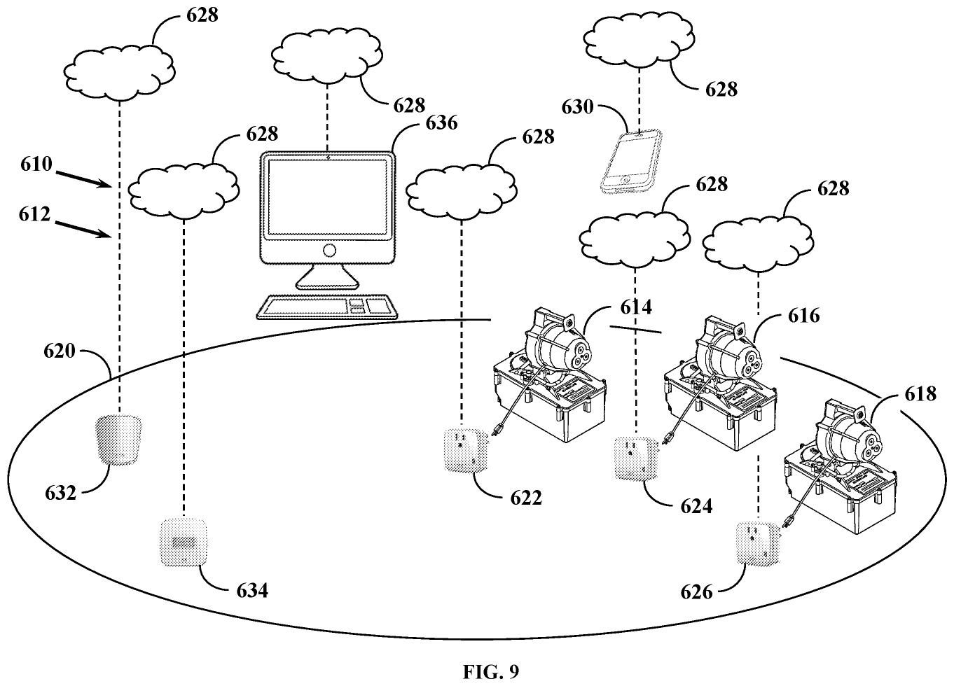

In some embodiments, and as illustrated in FIG. 9, the sequential application and delivery system 610 comprises an Internet-based Internet of Things (IoT) 612 used to control the dispensing of the liquid compositions from one or more sprayers 614, 616, and 618 located within a volumetric space 620. An Internet-based IoT 612 is particularly suited to those embodiments where wireless connectivity between various devices, e.g., outlets, sensors, etc., within the system 610 and the Internet is readily obtained from within the volumetric space 620, in situations or circumstances where a lesser degree of robustness in the system 610 can be tolerated, or when manual access by a human to the spraying equipment and its controls is unsafe, compromised, or otherwise prevented by either the identity of the compositions themselves or by the layout of the volumetric space itself.

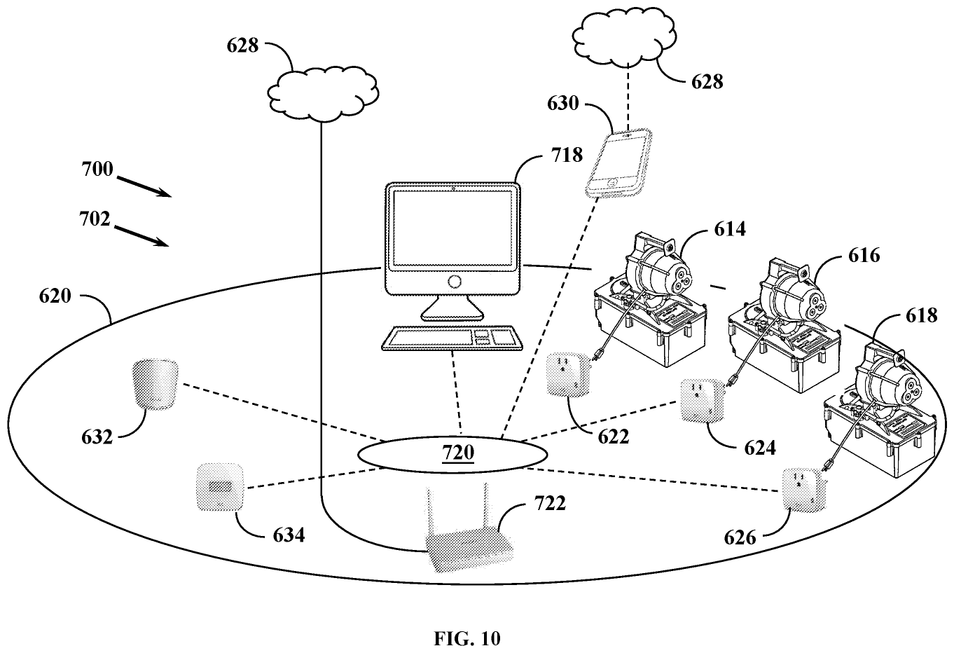

Similarly, in other embodiments, and as illustrated in FIG. 10, a sequential application and delivery system 700 comprises an intranet-based IoT 702 used to control the dispensing of the liquid compositions from two or more sprayers 614, 616, and 618 located within a volumetric space 620. An intranet-based IoT 702 is particularly suited to those embodiments where wireless connectivity between various devices within the sequential application and delivery system 700 and/or access to the Internet is restricted or limited. One such non-limiting situation is when the volumetric space 620 is a metal shipping container. In other embodiments, an intranet-based sequential application and delivery system 700 can be utilized in situations or circumstances where a more robust communication between devices is required, relative to what an Internet-based sequential application and delivery system 600 can provide.

In some embodiments, the Internet-based IoT 612 or the intranet-based IoT 702, can be used to control the sequential, time-dependent application of liquid compositions using spray devices comprised within any of the sequential application and delivery systems 310, 410, or 510 described above, or as illustrated In FIG. 9 and FIG. 10 by sprayers 614, 616, and 618. In other embodiments, sequential application and delivery systems 610 and 700 can be used to control the sequential, time-dependent application of liquid compositions using commercially-available sprayers, such as, in a non-limiting example, Hurricane™ sprayers sold by Curtis Dyna-Fog, Ltd. Each Hurricane™ sprayer provides the ability to manually control the flow rate of the respective aqueous compositions with selectable settings of low, medium, and high flow rates. From the factory or in stock form, these settings correspond to flow rates of 6.4, 8.0, and 9.0 fluid ounces per minute (0.19, 0.24, and 0.27 liters per minute), respectively. However, the metering valves included in any other commercial sprayer or manufactured spray device, including Hurricane™ sprayers, can be modified or replaced to utilize any desired flow rate, which can be varied under control of the Internet-based IoT 612 or the intranet-based IoT 702 within sequential application and delivery systems 610 and 700, respectively.

In some embodiments, the Internet-based IoT 612 or the intranet-based IoT 702 can be utilized to control a single sprayer that sequentially dispenses each of the liquid compositions in a time-dependent manner, similar to the arrangement shown in FIG. 5 or FIG. 7. In other embodiments, the Internet-based IoT 612 or the intranet-based IoT 702 can be utilized to control two or more sprayers, illustrated by 614, 616, and 618 in FIG. 9 and FIG. 10, to sequentially dispense each of the liquid compositions in a time-dependent manner. The two or more sprayers 614, 616, and 618 can be arranged within a single manifold or as separately housed units as shown in FIG. 9 and FIG. 10. The two or more sprayers 614, 616, and 618 can be switched into the powered-on position and plugged into respective remotely controlled outlets 622, 624, and 626 which are also conveniently located within the volumetric space 620. In turn, the remotely controlled outlets 622, 624, and 626 can be plugged into an electric power distribution system (not shown). In embodiments in which an intranet-based IoT 702 is used in conjunction with sequential application and delivery system 700, the remotely controlled outlets 622, 624, and 626 can be co-located with a hub 718, as illustrated in FIG. 10, particularly where wireless access is restricted.

In some embodiments, the hub 718 can be one of a number of suitable machines and/or devices, encompassing everything from a personal computer 718, as shown, to a NAS device. Non-limiting examples further include a laptop, desktop, or tower type machine, a tablet, or Apple TV™, Apple HomePod™, Amazon Alexa™ or Echo™, Google Home™, and a single board computer (SBC), such as a Raspberry Pi™. The hub 718 is typically located inside the volumetric space 620, and can be in electronic communication with the Internet wirelessly through WLAN 720 and, in turn, wired, as indicated by the solid line extending from the access point and/or router 722 to the Internet or cloud 628.

In some embodiments, the hub 718 typically operates using an operating system such as, for example, Android™, Android Oreo™, Apple® iOS®, Apple® OS X®, macOS®, or Apple® iOS®, Linux™, or any one of a number of Microsoft® Windows® operating systems, such as the currently active families of Windows® NT and Windows® Embedded, encompassing the subfamilies of Windows® CE and Windows® Server.

Those skilled in the art would appreciate that FIG. 9 and FIG. 10 only show three sprayers 614, 616, and 618, as well as three remotely controlled outlets 622, 624, and 626 for clarity, and that the sequential application and delivery systems 610 and 700 can be configured to control any number of sprayers plugged into any number of remotely controlled outlets, depending on variables such as configuration of the volumetric space, volume of liquid composition on hand, desired coverage of the liquid composition on surfaces within the volumetric space, atmospheric conditions, and power limitations, as non-limiting examples.

In some embodiments, the two or more sprayers 614, 616, and 618 and the remotely controlled outlets 622, 624, and 626 can be configured for use in any worldwide electric power distribution system. As a non-limiting example, an electric power distribution system can provide between 110-130 or 220-250 volts alternating current (VAC). In another non-limiting example, the remotely controlled outlets 622, 624, and 626 are configured to support appliances up to 1,800 watts at 120 VAC, 60 Hertz (Hz), 15 amperes (A), such as the two or more sprayers 614, 616, and 618.

In other embodiments, power cords from the two or more sprayers 614, 616, and 618 within the volumetric space 620 can extend out of the volumetric space 620 and plugged into one or more remotely controlled outlets 622, 624, or 626 located outside of the volumetric space 620. In one non-limiting example where the volumetric space 620 is a metal shipping container that has no internal access to the power grid, power cords from sprayers 614, 616, and 618 can extend through an opening separating the shipping container from the external environment and plugged into one or more remotely controlled outlets 622, 624, or 626 that are located outside of the shipping container.

In some embodiments, each of the remotely controlled outlets 622, 624, and 626 can generally comprise a relay and an associated wireless control for energizing or actuating the relay. In some embodiments, the relays can be of the mechanical or solid-state type. In further embodiments, the remotely controlled outlets 622, 624, and 626 can additionally comprise a relay driver circuit or transistor that provides the necessary power for energizing or actuating the relay. The wireless control allows remote actuation of the relays to switch or pass electric power from the electric power distribution system through the remotely controlled outlets 622, 624, and 626 to energize the respective two or more sprayers 614, 616, and 618, that are plugged into the remotely controlled outlets 622, 624, and 626.

The remotely controlled outlets 622, 624, and 626 are further configured for global accessibility with the Internet using wireless local area networking based on the Institute of Electrical and Electronics Engineers (IEEE) 802.11 standards, i.e., WiFi®, in the 2.4 Gigahertz (GHz) and/or the 5.8 Gigahertz (GHz) super high frequency (SHF) industrial, scientific, and medical (ISM) radio bands. In the sequential application and delivery systems 610 and 700, the remotely controlled outlets 622, 624, and 626 wirelessly connect with the cloud 628 as shown in FIG. 9 and FIG. 10, respectively, with wireless connectivity being generally indicated by dashed lines.

The remotely controlled outlets 622, 624, and 626 can also be further configured to operate or work with one or more of a number of readily available commercial home automation software packages available for use with one or more of several operating systems, including mobile operating systems. The commercial home automation software packages include Amazon Alexa™, Apple HomeKit™, Google Assistant™, Nest®, and Wink®, to name but a few. The operating systems include, but are not necessarily limited to, Apple® OS X® or macOS®, Linux™, and any one of a number of Microsoft® Windows® operating systems, such as the currently active families of Windows® NT and Windows® Embedded, which encompass the subfamilies of Windows® Embedded Compact (Windows® CE) and/or Windows® Server. The mobile operating systems generally include, but are not necessarily limited to, Android™, Android Oreo™, and Apple® iOS®.

The remotely controlled outlets 622, 624, and 626 can also be used with open source home automation software including, for example, Calaos, Domoticz, Home Assistant, OpenHAB (short for Open Home Automation Bus), and/or OpenMotics. Calaos is designed as a full-stack home automation platform, including a server application, touchscreen interface, web application, native mobile applications for iOS® and Android™, and a preconfigured Linux™ operating system which runs underneath. Domoticz is written in C/C++ and designed with an HTML5 frontend, is accessible from both desktop browsers as well as most modern smartphones, and is lightweight, running on many low power devices like, for example, a Raspberry Pi™. Home Assistant is an open source home automation platform, and is designed to be easily deployed on most any machine that can run Python® 3, from a Raspberry Pi™ to a network attached storage (NAS) device, and includes a docker container to facilitate deploying on other systems. Home Assistant also integrates with a number of other open source and commercial offerings. OpenHAB® is written in JAVA® and is portable across the major operating systems and can be configured to run on a Raspberry Pi™ as well. OpenHAB® also includes Android™ and iOS® applications for device control, and design tools for creating a user interface (UI). OpenMotics is a home automation system with both hardware and software, however, it is focused more on hardwired compositions.

In some embodiments, sequential application and delivery systems 610 and 700 can further optionally comprise one or more sensors as described by sensor 344ₓ above, shown in FIG. 9 and FIG. 10 as 632 and 634. Sensors 632 and 634 can likewise be configured for use and in wireless electronic communication with the Internet or intranet through WiFi® or a WLAN based on IEEE 802.11 standards in the 2.4 and/or 5.8 GHz SHF ISM radio bands.

In some embodiments, an IoT-based sensor in accordance with principles of the present invention can be designed and constructed to connect to the Internet, intranet, or cloud 628, and includes modules for Bluetooth® Low Energy (BLE), sub-GHz radio frequency (RF), and WiFi®, along with a dynamic near field communication (NFC) integrated circuit, a printed antenna, and a microcontroller on a single circuit board. Such IoT-based sensors and/or components for making them are commercially available from STMicroelectronics®, among others.

In some embodiments, sequential application and delivery systems 610 and 700 can further comprise an IoT door lock that is installed on a door that can selectively restrict access to the volumetric space 620. In further embodiments, sequential application and delivery systems 610 and 700 can be configured to actuate the IoT door lock to limit or prevent human access to the volumetric space 620 as the liquid compositions are being applied for a user-defined period of time.

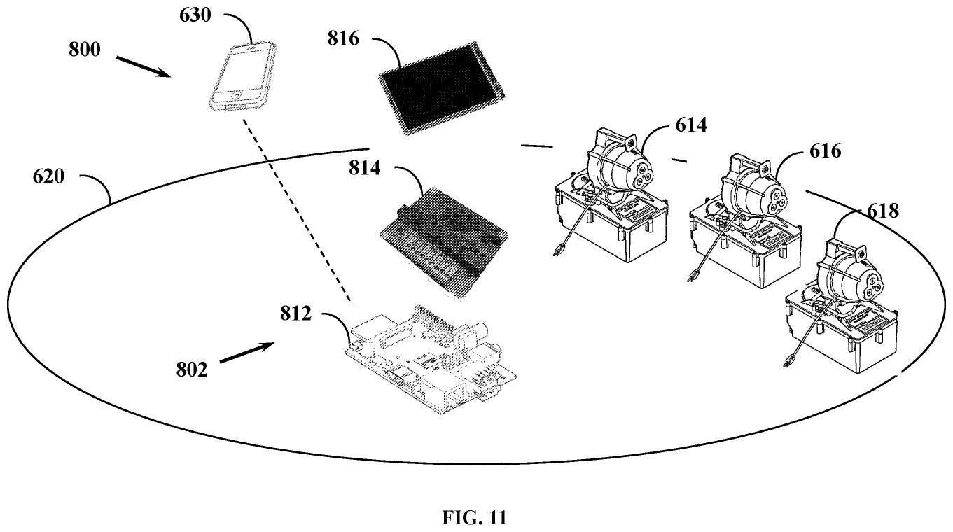

In some embodiments, as illustrated in FIG. 11, a sequential application and delivery system 800 can comprise a single board computer (SBC) assembly 802 used to control the dispensing of the aqueous compositions from two or more sprayers 614, 616, and 618 located within a volumetric space 620. The SBC assembly 802 is comprised of an SBC 812, an add-on circuit board or Hardware Attached on Top (HAT) 814, and an optional screen or display 816. In further embodiments, a sequential application and delivery system 800, in conjunction with an SBC assembly 802, can be utilized in a volumetric space 620 in which wireless connectivity with the Internet is precluded, limited, or undesired. In other further embodiments, embodiments, as a non-limiting example, sequential application and delivery system 800 can be utilized harsh or hazardous industrial environments in which other sequential application delivery systems can become damaged. In even further embodiments, a programmable logic controller (PLC) can be substituted for the SBC assembly 802 without departing from the spirit of the present invention.

In some embodiments, a HAT 814 can function as a “plug-n-play” add-on board for an SBC that conforms to a specific user- or hardware-defined set of rules and performs a wide variety of different functions, including, but not limited to, power control. In one non-limiting example, the HAT 814 conforms to a specific set of rules associated with a Raspberry Pi™ 3 40-pin general purpose input/output (GPIO) header connector. The HAT 814 circuit board carries or comprises a number of relays that the power inlets (power cords) of the two or more sprayers 614, 616, and 618 can be wired to in order to apply power in a sequential timed manner to the two or more sprayers 614, 616, and 618. Several suitable power relay HATs are currently and widely available, any of which can be configured for use with any number of different SBCs. A non-limiting example of a suitable power relay HAT is a Raspberry Pi™ four-channel relay HAT.

In some embodiments, one or more sprayers can be switched on and plugged into respective digitally-controlled outlets on one or more controllable four-outlet power relay modules located within the volumetric space 620, which can in turn be plugged into an electric power distribution system (not shown). The controllable four-outlet power relay modules can be controlled using a two-wire interface, i.e., serial parallel interface (SPI) or Inter-Integrated Circuit (I2C), by the SBC 812.

In various embodiments, the HAT 814 or the one or more controllable four-outlet power relay modules and the two or more sprayers 614, 616, and 618 can be configured for use in electric power distribution systems that provide between 110-130 or 220-250 VAC. For example, and in some embodiments, the HAT 814 and the one or more controllable four-outlet power relay modules are configured to support appliances up to 1,800 watts at 120 VAC, 60 Hz, 15 A, e.g., the two or more sprayers 614, 616, and 618.

In some embodiments, the SBC 812 can include on-board WiFi® capability, along with a number of other connectivity options and/or functions, such as, for example, a High-Definition Multimedia Interface (HDMI), composite video, a Uniform Serial Bus (USB) 2.0, General Purpose Input/Output (GPIO), I2C, and Ethernet®, as will be readily understood by a person of ordinary skill in the art. Other non-limiting exemplary models of a Raspberry Pi™ that can also be used include the: Raspberry Pi™ 1 Model B, Raspberry Pi™ 1 Model B+, Raspberry Pi™ 2, Raspberry Pi™ Zero, Raspberry Pi™ 3 Model B, Raspberry Pi™ 3 Model B+, and the Raspberry Pi™ Zero W. In other embodiments, other types of SBC 812 can also be used as desired without departing from the spirit of the present invention. Non-limiting examples of other SBCs include: the Asus™ Tinker; armStone; Arndale; Arndale Octa; Banana Pi including the Pro, M2, and M3; BeagleBoard® including the xM; BeagleBone®; CubieBoard; Firefly™; NanoPi and NanoPi NEO; ODROID including the C1, C1+, C2, U3, W, XU, XU3, XU3 Lite, and XU4 models; Orange Pi including the Pi, Pi2, Pi Plus, Pi Plus 2, Pi Mini, Pi Mini 2 PC, One, Lite, PC Plus, Plus 2E, PC 2, Pi Win, and Pi Zero Plus 2; and the pcDuino® including the Lite, v2, 3, and 3 Nano models.

In some embodiments, the SBC 812 can be configured to run in access point (AP) mode. AP mode is particularly advantageous in that it allows wireless devices to connect directly to the SBC 812 using WiFi® based on IEEE 802.11 standards in the 2.4 and/or 5.8 GHz SHF ISM radio bands for control purposes, without having to have or use a wired or wireless network. Further, and in some embodiments, AP mode allows the SBC 812 to run “headless,” or without a screen.

In some embodiments, operational control of sequential application and delivery systems 610, 700, and 800 can be performed using a home automation application installed on a mobile device 630, an electrically connected remote computer 636, a hub 718, or on a display 816.

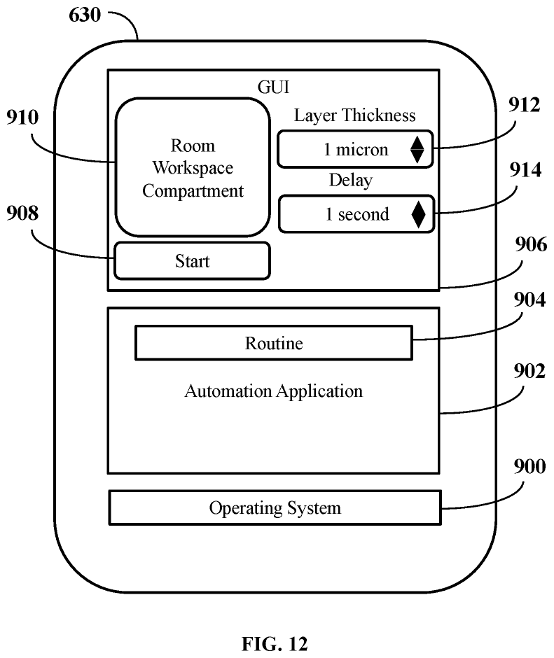

In some embodiments and as illustrated in FIG. 12, home automation application 902 is installed on mobile device 630 comprising a programmed or programmable controller. Non-limiting examples of suitable mobile devices include a handheld computer, a smartphone, smartwatch, tablet, iPad®, laptop, personal digital assistant (PDA), portable media player, or personal navigation device.

In some embodiments, the mobile device 630 can be located outside of the volumetric space 620. When located outside of the volumetric space, the mobile device 630 can be in wireless electronic communication with the Internet or cloud 628 either through WiFi®, a wireless local area network (WLAN) based on IEEE 802.11 standards in the 2.4 and/or 5.8 GHz SHF ISM radio bands, or through a cellular telephony network using analog or digital modulations schemes, e.g., Advanced Mobile Phone System (AMPS), or Code Division Multiple Access (CDMA) or Global System for Mobile Communications (GSM), in the ultrahigh frequency (UHF) band, i.e., 300 MHz to 3 GHz, that have been assigned for cellular compatible mobile devices, such as mobile phones or smartphones. The wireless capability of the mobile device 630 allows its user to easily remain outside of the volumetric space 620 and avoid contact with the liquid compositions.

In some embodiments, the mobile device 630 utilizes a mobile operating system 900, non-limiting examples of which include Android™, Android Oreo™, and Apple® iOS®. The installed home automation application 902 on the mobile device 630 can include a commercial, open source, or user-programmed software package. Non-limiting examples of a commercial home automation software packages include, but are not necessarily limited to, Amazon Alexa™, Apple HomeKit™, Google Assistant™, Nest®, and Wink®, while non-limiting examples of an open source home automation software package include, but are not necessarily limited to, Calaos, Domoticz, Home Assistant, OpenHAB®, and OpenMotics. A person having ordinary skill in the art will appreciate that other software providing a basis for automation, including other operating systems and commercial and/or open source software, could also be used without departing from the spirit of the present invention.

In some embodiments, a routine 904 can be programmed within the home automation application 902 to recognize, monitor, and control devices within the volumetric space 620. As applied to the sequential application and delivery system 610 shown in FIG. 9, a routine 904 can be utilized to energize remotely controlled outlets 622, 624, and 626, connected to sprayers 614, 616, and 618, respectively, in a sequential timed manner. For example, routine 904 can be programmed to actuate a first remotely controlled outlet 622 to energize a first sprayer 614 for a first period of time (t₁), causing the first sprayer 614 to dispense a first liquid composition into the volumetric space 620. After a delay (d₁) for the first liquid composition to distribute throughout the volumetric space 620 and deposit and coalesce into a layer upon one or more surfaces within the volumetric space 620, the routine 904 can actuate a second remotely controlled outlet 624 to energize a second sprayer 616 for a second period of time (t₂) causing the second sprayer 616 to dispense a second aqueous composition into the volumetric space 620. In some embodiments, from a graphical user interface (GUI) perspective, initiation of the routine 904 can be accomplished simply by pressing a single button 908, labelled “Start,” in one non-limiting example.

Precise control of the amount of time that a composition is dispersed, the flow rate that a composition is dispersed, and the delay between dispersing compositions, has several advantages, including, but not limited to, dispersing a stoichiometric amount of the liquid composition, avoiding application of excess volumes of the liquid composition, ensuring that the composition has contacted and formed a layer on all of the intended surfaces, and confirming that the desired interaction between two or more compositions has had adequate time to take place. In some embodiments, precisely controlling the delays d₁ and d₂ ensures that the liquid compositions are dispersed sequentially, and not simultaneously, onto the target surfaces. In other embodiments, control of the sequential application and delay prevents unwanted reactions from occurring within the volumetric space before the components within the aqueous compositions reach the surface.

In some embodiments, the periods of time for spraying and associated delays between sprays can be calculated within the home automation application. In other embodiments, the periods of time for spraying and associated delays between sprays can be empirically determined by the user. A person of ordinary skill in the art will appreciate that the periods of time for spraying and the associated delays between sprays can be adjusted as required based one or more variables, non-limiting examples of which include the characteristics of the volumetric space 620, the components within one or more of the aqueous compositions, and the surface(s) or substrate(s) upon which the aqueous compositions are deposited.

In some embodiments, from a GUI perspective, an environment selection 910 can be made by a user within the home automation application 902 that inputs data relating to a specific type of environment, i.e., volumetric space 620, that is, in turn, used by routine 904. In some embodiments, the environment is a confined space, isolated from other areas and spaces by walls, ceilings, or other barriers. Such examples of environments include, but are not necessarily limited to, a “Room,” a “Workspace,” and a “Compartment.” In other embodiments, the airspace within the environment can be immobilized from access to other environments. In one non-limiting example, air vents for a heating, ventilation, and air conditioning system that are present within a volumetric space 620 can be accessed and blocked off to prevent any of the dispersed aqueous compositions from encroaching adjacent volumetric spaces or environments during the routine 904.

In some embodiments, sensors 632 and 634 utilized in conjunction with an IoT 612 or 702 can be programmed to be recognized, monitored, and/or controlled by the home automation application 902. In further embodiments, information about the volumetric space 620, a non-limiting example of which includes room dimensions, can be pre-loaded into the mobile device 630 either through an interface, for example, the GUI 906 shown in FIG. 12, or through a similar interface on an electrically connected remote computer 636, hub 718, or display 816.

In some embodiments, the routine 904 can additionally comprise a means for determining, calculating, and/or selecting an effective uniform thickness of a coalesced layer of a liquid composition to dispense on surfaces within the volumetric space 620, such as, for example, through a drop-down layer thickness selection pane 912 on GUI 906.

In some embodiments, the routine 904 can utilize information determined or estimated by the one or more sensors prior to dispensing, including the size of the volumetric space 620, the relative humidity within the volumetric space 620, and/or the desired effective uniform thickness of the coalesced layer, to determine the appropriate volume of the aqueous compositions to dispense in order to contact all of the intended surfaces with the desired amount of each aqueous composition. In use, calculations made or performed by the routine 904 based on pre-programmed data or information detected by the one or more sensors can specify a specific quantity, rate, and/or time to dispense a particular aqueous composition, and can implement a calculated or pre-programmed time delay between dispensing the first liquid composition, the second liquid composition, and any other liquid compositions. Additionally, the routine 904 can be programmed to select from one or more optional pre-programmed routines, including routines in which a composition consisting essentially of water or other inert, non-reactive materials is dispersed prior to dispersing the first liquid composition, after dispersing a first liquid composition and before a second liquid composition, or after dispersing the liquid aqueous composition, using for example, the sprayer 618 and the respective remotely controlled outlet 626.

In other embodiments, the routine 904 can additionally calculate and determining the time sufficient for a liquid composition to dispense, distribute throughout the volumetric space 620, and coalesce into a layer on the desired surfaces, before dispersing a succeeding liquid composition. In some embodiments, a user can select or input a desired time for the routine 904 to wait before dispersing the succeeding aqueous composition, such as, for example, through a drop-down selection panel 914 shown in FIG. 12. In other embodiments, the routine 904 can use the determined size of the volumetric space or the area and/or volume of the liquid composition required in order to calculate the time sufficient for a layer to coalesce onto a layer on the desired surfaces before dispensing the succeeding liquid composition.

In another embodiment, the routine 904 can use data from the sensors 632 and 634 from within the volumetric space 620 for determining the time sufficient for a liquid composition to arrive and coalesce into a layer on surfaces within the area. As a non-limiting example, one or more sensors can be placed in desired locations and/or surfaces within the volumetric space 620, whereupon the one or more sensors communicate to the routine 904 when the liquid composition comes into contact with the sensor. In further embodiments, one or more sensors placed throughout the volumetric space 620 must be contacted by a dispersed liquid composition in order to communicate to the routine 904 to initiate a delay period before dispersing a succeeding liquid composition.

In some embodiments, the routine 904 can be programmed so that the routine can only be initiated by a device operated by a user outside of the volumetric space 620. In other embodiments, the routine 904 can be programmed so that one or more of the liquid compositions are only dispersed when the volumetric space 620 is completely empty of any people or animals, as determined by one or more sensors 632 or 634 located within the volumetric space 620, or GPS capabilities inherently programmed into the device. In still other embodiments, the routine 904 can be initiated while a person and/or the mobile device, computer, hub, or display operating the routine 904 is located within the volumetric space 620.

In some embodiments, after a routine 904 has been initiated by a home automation application 902, the routine 904 can be programmed to be terminated if movement within the volumetric space is detected by a particular sensor, or by comparison of the GPS position of a mobile device running the routine with the GPS position of the volumetric space. In further embodiments, upon detection of movement within the volumetric space 620, the routine 904 can be programmed to initiate the application of water or some other inert substance to “scrub” the air within the volumetric space 620 to dilute or remove potentially hazardous chemicals within the liquid compositions from remaining in the airspace. In other further embodiments, movement within the volumetric space 620 during routine 902 can trigger a notification or alert on the sequential application and delivery system 610, 700, or 800, on the mobile device 630 running the routine 904, or on a secondary device located outside of the volumetric space 620 that is not associated with running the routine. Non-limiting examples of notifications that can be sent to a secondary device include a text message or email.

In some embodiments, the notification or an alert is a message displayed on the GUI 906 indicating that the user should not enter the volumetric space 620, that the user should leave the volumetric space 620, and/or that it is safe to enter the volumetric spaces. In other embodiments, sequential application and delivery systems 610, 700, or 800 can programmed to illuminate a light located outside the volumetric space 620, for all persons to see, indicating that a routine 904 is in progress, that someone has entered the volumetric space 620, and/or that it is safe to enter the volumetric space. In further embodiments, visual notifications and/or alerts can include a “red” light indicating that a routine 904 is in progress and that a person should not enter the volumetric space or a “green” light indicating that the routine 904 has now finished, and that a person may now enter the volumetric space.

In some embodiments, the notification or alert is an auditory siren that sounds if a person or animal enters the volumetric space during the running of routine 904. In further embodiments, the auditory alert is a verbal warning telling the person to exit the volumetric space. Those skilled in the art will appreciate that systems 610, 700, or 800 can be configured to give any combination of visual, auditory, or other notifications and/or alerts, within any combination of colored lights, aural signals, or verbal messages, as desired without departing from principles of the present invention.

In some embodiments, either the aqueous compositions or the sequential application and delivery systems for dispensing the aqueous compositions can be packaged together as a kit. In some embodiments, a kit for use in disinfecting a surface in need of disinfecting within a volumetric space can comprise: a) a first aqueous composition comprising a first peracid reactant compound that is either a peroxide compound or an organic acid compound capable of reacting with a peroxide compound to form a peracid; b) a second aqueous composition comprising a second peracid reactant compound that is the other of the first peracid reactant compound; and c) instructions comprising any of the methods described above, wherein the kit is arranged such that the first aqueous composition and the second aqueous composition are packaged separately and are not combined until the first aqueous composition and the second aqueous composition are applied sequentially onto the surface to form a reaction layer comprising the first aqueous composition and the second aqueous composition, thereby forming a peracid in situ within the reaction layer and disinfecting the surface.

In some embodiments, kits comprising a sequential application and delivery system can additionally include one or more IoT or SBC devices described above to control the sequential application and delivery system and implement any of the chemical, disinfecting, or sterilization methods described above.

While particular embodiments of the invention have been described, the invention can be further modified within the spirit and scope of this disclosure. Those skilled in the art will recognize, or be able to ascertain using no more than routine experimentation, numerous equivalents to the specific procedures, embodiments, claims, and examples described herein. As such, such equivalents are considered to be within the scope of the invention, and this application is therefore intended to cover any variations, uses or adaptations of the invention using its general principles. Further, the invention is intended to cover such departures from the present disclosure as come within known or customary practice in the art to which this invention pertains and which fall within the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

The contents of all references, patents, and patent applications mentioned in this specification are hereby incorporated by reference, and shall not be construed as an admission that such reference is available as prior art to the present invention. All of the incorporated publications and patent applications in this specification are indicative of the level of ordinary skill in the art to which this invention pertains, and are incorporated to the same extent as if each individual publication or patent application was specifically indicated and individually indicated by reference.

The invention is further illustrated by the following examples, none of which should be construed as limiting the invention. Additionally, to the extent that section headings are used, they should not be construed as necessarily limiting.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. Thus, while the present invention has been described above with particularity, the following examples provide further detail in connection with what are presently deemed to be the most practical and preferred embodiments of the invention.

### Example 1: Closed-System Electrospray Distribution Study

A study was conducted in accordance with embodiments of the present disclosure to evaluate the distribution of an aqueous composition containing 5% by weight acetic acid onto multiple target surfaces using an electrostatic spray device. Two analytical balances were placed inside a 1 cubic meter, transparent glove box (the “Cube”) and connected to a computer station configured to collect and record mass measurements as a function of time. Each balance had a standard reading error of 0.005 grams. On each balance, a 1000 square centimeter plastic sheet was placed inside a weighing pan. The position of each balance was staggered to be in different positions along the x, y, and z axes in relation to the electrostatic sprayer, placed at one end of the Cube.

The Cube was constructed with an external framework of wood covered on the inside with clear vinyl. The floor of the Cube was white Formica. An ante-chamber was placed on the lower portion of one of the walls of the Cube. There was an exhaust fan in the ante-chamber. Another wall of the Cube housed a door that enabled the entire wall of the Cube to be opened like a door. Makeup air when the Cube was being exhausted was provided through a portal on an upper corner on the ceiling of the Cube and adjacent to the wall opposite of the ante-chamber. The portal was covered with a HEPA filter that used a high efficiency furnace filter as a pre-filter. In order to manipulate materials inside the Cube while the Cube was closed to the outside environment, a single glove was installed on the wall opposite of the ante-chamber, and two gloves were installed adjacent to the ante-chamber itself. Shelves were installed near each glove station to enable the placement of the balances at staggered x, y, and z, positions, as described above. A digital thermometer and humidity meter were also installed inside the Cube.

The electrostatic spray device used was a Hurricane ES™ Portable Electrostatic Aerosol Applicator, which was placed inside the ante-chamber of the Cube. The makeup air for the sprayer came from the Cube and passed under the Sprayer so it could enter the back of the sprayer. This air was forced through the sprayer where it picked up the test solution and was forced through three nozzles in the path of three electrodes. The spray then passed through a short chamber containing a high intensity UV C light before passing into the Cube. The test solution feed line exited the ante-chamber and extended into a beaker seated on an analytical balance. About 24.5 grams of each test solution were passed into the Cube, giving a theoretical effective film thickness of about 3 microns. Objects to be tested were placed outside of the direct line of the sprayer so they only received an indirect spray, mimicking potential conditions of a surface to be disinfected in practice. During each experiment, all openings for the Cube were sealed from the outside environment.

The acetic acid composition was then electrostatically sprayed throughout the entire Cube for 30 seconds with a set particle size of about 15 microns. The time of application was selected to provide a 2-micron thick coating within the treatment space as measured by the balances. During the application, mass measurements from the two balances were collected and recorded by the computer. The result of the test is provided as follows:

TABLE 2
Electrospray Distribution

| | Mass—First |
|---|---|
| | Aqueous Composition (g) |
| Balance A (with 1000 cm² plate) | 0.205 +/− .005 |
| Balance B (with 1000 cm² plate) | 0.190 +/− .005 |

The mass of the first aqueous composition deposited on balance A and balance B indicated a difference of 0.015+/−0.010 grams. In combination with a qualitative observation that the inside surfaces of the Cube appeared to be equally coated with the acetic acid solution, it is believed that the electrospray method evenly distributed the first aqueous composition within the Cube.

### Example 2: Preparation of First and Second Aqueous Compositions

Two separate aqueous compositions containing a peracid reactant compound, one containing acetic acid and one containing hydrogen peroxide, were prepared in accordance with embodiments of the present disclosure, which includes the following ingredients in approximate amounts.

### First Aqueous Composition:

8% (w/w) Acetic Acid
15% (w/w) Ethanol
0.003% (w/w) Cinnamon Oil
76.997% (w/w) Distilled Water

### Second Aqueous Composition:

5% (w/w) Hydrogen Peroxide
15% (w/w) Ethanol
80% (w/w) Distilled Water

The first aqueous composition and second aqueous composition were placed in separate containers until they could be dispersed on to surfaces in need of disinfecting within a volumetric space.

### Example 3: Closed-System Log-Kill Studies by Sequential Addition of the Aqueous Compositions of Example 2

A study was conducted in accordance with embodiments of the present disclosure to determine the antimicrobial activity against common strains of bacteria by sequentially applying the two aqueous compositions of Example 2 to form peracids in situ directly on surfaces to be disinfected within a closed system. The closed system was the Cube used in Example 1. Cultures from commercially-available strains of four species of bacteria-Bacillus subtilis, Micrococcus luteus, Rhodospirillum rubrum, and Staphylococcus epidermis-were selected for a log-kill study because they possess several known defense mechanisms to common biocides while at the same time having different physical properties from each other. Sterilized, pre-poured agar plates were used as growth media to produce colonies of each bacteria. 8 plates were inoculated for each species. Of those 8 plates, 4 plates were exposed to the sequential application of the two aqueous compositions of Example 2, and 4 plates were held out as controls. Plates were inoculated using the standard T-method of streaking for log-kill studies, where the concentration of bacteria in the fourth quadrant of the plate is about 1,000,000× diluted with respect to the first quadrant. The test plates for each species were then placed inside the Cube with the lids open. Control plates were sealed with tape.

Upon closing the Cube, a multiplicity of microdroplets of the first aqueous composition was electrostatically applied to the entire Cube using a Hurricane ES™ Portable Electrostatic Aerosol Applicator. Microdroplets were sprayed for 30 seconds, using a flow rate of 6 oz./min, which correlates with a microdroplet size of 10-20 microns, according to the instructions provided by the manufacturer of the Hurricane ES™ applicator. The timing of the application of the first aqueous composition was selected to provide a coating having a calculated 2-micron thickness on the plates within the treatment space, as determined by the mass of the solution. About 1 minute after completing the spraying of the first aqueous composition, the second aqueous composition was sprayed for 3 seconds at a distance of about 6-8 inches using a hand sprayer, and the entire system was untouched for another 5 minutes. After evacuating the airspace of residual spray, the test plates were closed with their lids inside the Cube before being brought out into the ambient environment, where they were sealed with tape. During the transfer from the Cube to the outside environment, the lids of the B. subtilis test plates 1 and 2 were inadvertently opened. These plates were immediately closed and sealed with tape. All of the sealed test and control plates were then incubated at about 28° C. and inspected after 1, 2, and 4 days.

The results of the tests are provided as follows:

TABLE 3
Presence of colonies after 1 day (+ or −)

| Plate Number | B. subtilis | M. luteus | R. rubrum | S. epidermis |
|---|---|---|---|---|
| 1 | + | − | − | − |
| 2 | + | − | − | − |
| 3 | − | − | − | − |
| 4 | − | − | − | − |

TABLE 4
Presence of colonies after 2 days (+ or −)

| Plate Number | B. subtilis | M. luteus | R. rubrum | S. epidermis |
|---|---|---|---|---|
| 1 | + | − | − | − |
| 2 | + | − | − | − |
| 3 | − | − | − | − |
| 4 | − | − | − | − |

TABLE 5
Presence of colonies after 4 days (+ or −)

| Plate Number | B. subtilis | M. luteus | R. rubrum | S. epidermis |
|---|---|---|---|---|
| 1 | + | − | − | − |
| 2 | + | − | − | − |
| 3 | − | − | − | − |
| 4 | − | − | − | − |

All controls produced the expected results, with positive control plates not treated with the sequentially-applied aqueous compositions containing the peracid reactant compounds showing growth for each organism characteristic of its growth within an open environment. Over the 16 control plates, there was an average of 4 colonies in the fourth quadrant of the plate, indicating that there were 4,000,000 colonies in the initial inoculation.

Colonies were observed on two B. subtilis test plates after 1 day. However, these test plates were the ones that were inadvertently exposed to the ambient environment after the method was completed, but before the lids were sealed. These colonies possessed a different morphology than those on the B. subtilis control plates. Consequently, it is believed that these colonies represent a false positive, based on bacteria that were introduced onto the plates when the lids were inadvertently opened. Because colonies were found on plates that had previously been exposed to a peracid, these results also suggest that the test plates themselves were capable of supporting bacterial growth, and that the lack of observable colonies on the rest of the test plates is a direct consequence of the disinfection method employed in the experiment. Therefore, the lack of colonies on the rest of the test plates, coupled with the approximately 4,000,000 colonies observed on the control plates, indicates that the method was effective to at least a log-6 kill rate, representing a kill of at least 99.9999% of the bacteria originally present on the plates.

### Example 4: Medium-Sized Volumetric Space Electrospray Distribution Study

A study was conducted in accordance with embodiments of the present disclosure to evaluate the distribution of an aqueous composition containing 1% by weight acetic acid onto multiple target surfaces using an electrostatic spray device. The electrostatic spray device used was a Hurricane ES™ Portable Electrostatic Aerosol Applicator. The laboratory space in which the testing surfaces were located was closed off to the surrounding environment and had a volume of about 30 cubic meters, approximately the size of a small hospital room. The electrospray device was placed on a platform approximately 2-feet high and approximately 5 feet from one of the corners of the laboratory space, and was pointed to face the opposite corner, enabling testing of distribution behind the electrospray device along the y-axis (defined below). Several pH testing strips were fixed throughout the laboratory space, particularly walls, floor, ceiling, and equipment, including exposed and non-exposed surfaces. The pH strips were evaluated both prior to and after electrospraying the acetic acid composition for a change in color in response to being exposed to the acetic acid composition. Each application of the acetic acid composition was sprayed with a negative charge.

For each application, the acetic acid composition was sprayed for approximately 45 seconds using a flow rate of 6 oz./min, which correlates with a microdroplet size of 10-20 microns, according to the instructions provided by the manufacturer of the Hurricane ES™ applicator. After spraying finished, researchers entered the room to evaluate the pH strips. Over three trials, every pH strip exhibited a color change during each trial, indicating that the acetic acid composition contacted each strip, even pH strips that were hidden or unexposed.

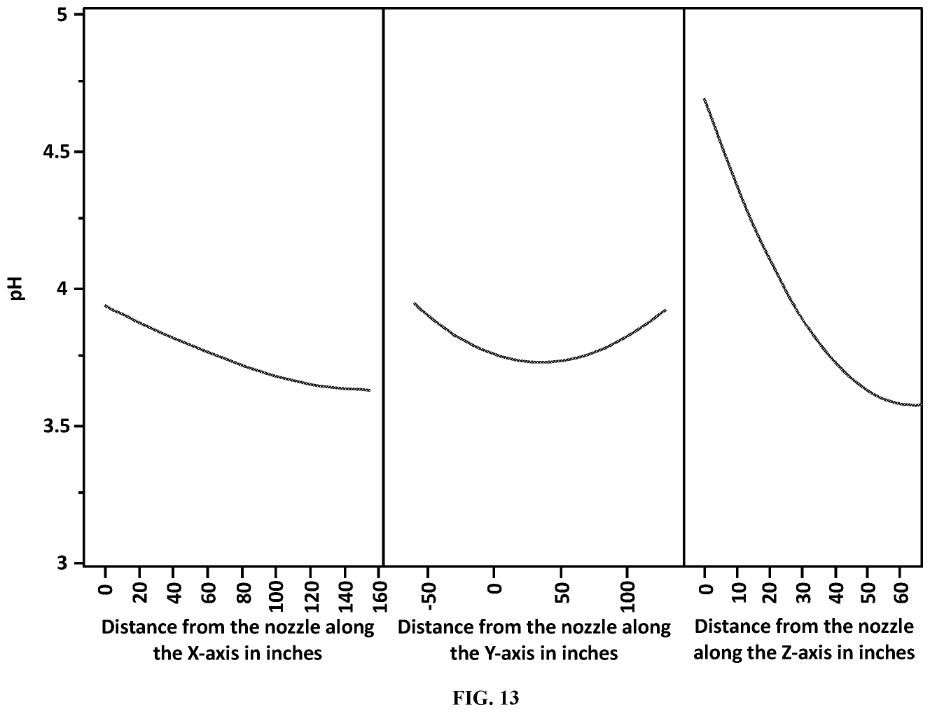

The pH at each pH strip location was quantified, and the pH distribution as a function of changes in x, y, and z direction from the nozzle on the electrospray device are shown in FIG. 13. Each of the lines represent a line of best fit of data collected from each of the pH strips within the area. A lower pH value indicates that more acetic acid contacted the pH strip at that location than at a location with a higher pH value. All distances were calculated in inches. The x-axis was defined as the horizontal axis perpendicular to the outward direction of the electrospray device. The y-axis was defined as the horizontal axis parallel to the outward direction of the electrospray device. The nozzle of the electrospray device was oriented to spray at a 45° angle relative to both the x- and y-axes. The z-axis is the vertical height extending directly upward or downward from the nozzle of the sprayer. Over both the x- and z-axes, contact by the acetic acid spray generally increased as the distance from the sprayer increased, as evidenced by the decreased pH measured at those locations. However, the effect was hyperbolic and flattened out after a time. Along the y-axis however, coverage generally decreased at a further distance away from the sprayer, although approximately the same decrease was observed both in front of (positive distance values) and behind (negative distance values) the electrospray. Nonetheless, in all cases, the difference between the pH at the greatest coverage and least coverage at the measured locations was narrow, although the effect was more pronounced along the z-axis.

### Example 5: Multidimensional Analysis of Reaction Parameters and their Effect on the Percent Kill of Bacteria

A study was conducted in accordance with embodiments of the present disclosure to evaluate the effect of several reaction parameters on the percent kill of microbes. Reaction parameters tested include: the concentration of the peracid reactant compounds in an aqueous composition, order of addition of aqueous compositions containing peracid reactant compounds, the charge applied when dispersing peracid reactant compounds, the concentration of alcohol included in each aqueous composition, the concentration of a natural biocide or biocidal compound included in each composition, and the effect of illuminating the surface with a wavelength consisting essentially of ultraviolet light. In all experiments in which an alcohol was included in an aqueous composition, the alcohol was ethanol. In all experiments in which a natural biocide was included, the natural biocide was cinnamon oil. Typical stock solutions used in the formulation of aqueous compositions for each experiment included distilled water, 35% food-grade hydrogen peroxide, 99% glacial acetic acid, 95% ethanol, and cinnamon oil diluted to 20% concentration with ethanol.

All experiments were conducted in the Cube utilized in Example 1. The electrostatic spray device used was a Hurricane ES™ Portable Electrostatic Aerosol Applicator, modified to have the capability to disperse microdroplets having a negative charge, positive charge, or a neutral charge. Three different bacteria were tested in each experiment, Bacillus subtilis, Micrococcus luteus, and Staphylococcus epidermidis, according to the procedures of Example 3. In some experiments, a second modified Hurricane ES™ Portable Electrostatic Aerosol Applicator was used to disperse microdroplets of the second aqueous composition, instead of using a hand sprayer as in Example 3. The amount of bacterial kill was evaluated as a percent kill, rather than a log kill, to evaluate experiments where one or more reaction components were not included, facilitating analysis comparing results across all experiments. Petri dishes containing bacteria were graded 24 hours, 3 days, and 5 days after each experiment. Bacterial control reactions were conducted in parallel with each experiment, according to the procedures of Example 3. In order to ensure a constant relative humidity and to facilitate deposition of the microdroplets of each aqueous composition, a pre-treatment step was utilized in each experiment, where distilled water was sprayed using a neutral charge inside the Cube until the relative humidity inside the Cube registered 90% on the humidity meter.

Data for each experiment was compiled into JMP, a statistical analysis software too available from SAS Institute, Inc, which is able to analyze, model, and visualize data over several variables in order to determine correlations between variables over several dimensions. Particularly, percent kill was determined in two dimensions as a function of multiple data points collected for each reaction parameter. Using all of the compiled data, JMP software can then calculate a model that can be used to determine the effect on the percent kill of the bacteria both at untested concentrations or values for a single reaction parameter, as well as the effect of one reaction parameter on the ability of other reaction parameters within the system to affect the bacteria.

In a first set of experiments, the effect of the presence of hydrogen peroxide, acetic acid, ethanol, cinnamon oil, as well as illumination by ultraviolet light and dispersion of the aqueous compositions in the presence of an electric charge was determined. Thirteen separate reaction conditions were tested, according to Table 6, below. The value reported in the percent kill column represents the average percent kill of all three of the species of bacteria, with each experiment repeated in triplicate.

TABLE 6

| Exp# | Comments | % Kill | HP | AA | EtOH | UV | Charge | Cinn. |
|---|---|---|---|---|---|---|---|---|
| 1 | Control-No treatment | 0 | | | | | | |
| 2 | Comp 1: HP (−) \| Comp 2: AA (+) | 87 | x | x | | | x | |
| 3 | Comp 1: HP (−) \| Comp 2: AA (+) | 90 | x | x | | | x | |
| 4 | Comp 1: HP (+) \| Comp 2: AA (−) | 94 | x | x | x | x | x | x |
| 5 | Comp 1: HP (−) \| Comp 2: AA (+) | 96 | x | x | x | x | x | x |
| 6 | Comp 1: AA (+) \| Comp 2: HP (−) | 95 | x | x | x | x | x | x |
| 7 | Comp 1: AA (−) \| Comp 2: HP (+) | 92 | x | x | x | x | x | x |
| 8 | Comp 1: HP/H2O \| Comp 2: none | 72 | x | | | | | |
| 9 | Comp 1: AA/H2O \| Comp 2: none | 6 | | x | | | | |
| 10 | Comp 1: EtOH/H2O \| Comp 2: none | 0 | | | x | | | |
| 11 | Comp 1: UV/H2O \| Comp 2: none | 21 | | | | x | | |
| 12 | Comp 1: H2O (−) \| Comp 2: none | 27 | | | | | x | |
| 13 | Comp 1: Cinn./H2O \| Comp 2: none | 17 | | | x | | | x |

As indicated in Table 6, “x” illustrates that the component is present in the experimental condition; “HP”=5% by weight of hydrogen peroxide; “AA”=8% by weight of acetic acid; “EtOH”=16% by weight of ethanol; “UV”=surface is illuminated by ultraviolet light during the reaction conditions; “Charge”=at least one aqueous composition is dispersed with an electrostatic charge; and “Cinn”=0.1% by weight of cinnamon oil. “Comp 1” refers to the aqueous composition dispersed first, and “Comp 2” refers to the aqueous composition dispersed second. In parentheses, the electrostatic charge of the aqueous composition as it was dispersed is shown, where applicable. In experiments in which ethanol was present in the reaction conditions, ethanol was included in both aqueous compositions. In experiments in which cinnamon oil was present in the reaction conditions, cinnamon oil was added in the composition along with acetic acid. In experiments in which the surface was exposed to UV light, the procedures according to Example 1 were utilized. Experiments 2 through 7 represent reaction conditions in which a peracid reactant compound was included in each of the dispersed aqueous compositions, while Experiments 8 through 13 represent control reactions in which one or both of the peracid reactant compounds was omitted.

The results in Table 6 illustrate that in experiments in which both peracid reactant compounds are included (Experiments 2 through 7), the percent kill is demonstrably larger than in any of the Experiments 8 through 13 in which one or zero peracid reactant compounds is included. Furthermore, the percent kill of Experiments 8 and 9 together, where either hydrogen peroxide or acetic acid only are included, are noticeably less than in any of Experiments 2 through 7 where both compounds are included. This result demonstrates that a peracid is being formed on the surface and that the increased bacterial kill is a result of forming the peracid. Experiments 4 through 7, which alter the order of dispersion and charge associated with each aqueous composition, each illustrate similar percent kill results to each other. The reaction conditions in Experiments 4 through 7, particularly 4 through 6, do illustrate that at least one of the ethanol, UV, or cinnamon oil are having an increased effect on the percent kill relative to reactions in which those components are absent (Experiments 2 and 3).

In a second set of experiments, the effects of concentration of the peracid reactant compounds, ethanol, and cinnamon oil were studied as a function of the order of addition and electrostatic charge over the course of 174 separate experiments. In several reactions, the concentration of some reaction components was kept intentionally low in order to determine the effect of other reaction conditions. The tested concentrations of acetic acid ranged from 0 to 15% by weight of the aqueous composition; the tested concentrations of hydrogen peroxide ranged from 0 to 10% by weight of the aqueous composition; the tested concentrations of ethanol ranged from 0 to 16% by weight of the aqueous composition; and the tested concentrations of cinnamon oil ranged from 0% to 0.16% by weight of the aqueous composition.

Percent kill data from each experiment as a function of altering one or more of the reaction variables were compiled into the JMP program. Data from all 174 experiments were utilized to calculate a model for predicting the average kill over all reaction conditions and tested concentration ranges for each reaction component. The calculated model determined that there were nine statistically significant (R²=97%) independent variables that had an effect on the percent kill, including: the acetic acid concentration, the polarity of the charge of the second dispersed aqueous composition, cinnamon oil concentration, the presence and order of addition of the composition comprising hydrogen peroxide, hydrogen peroxide concentration, and whether the surface was illuminated with ultraviolet light. Additional terms, including the square of the order of addition of the composition comprising hydrogen peroxide, the square of the hydrogen peroxide concentration, and whether the surface was illuminated with ultraviolet light in conjunction with the addition of hydrogen peroxide, where also statistically relevant.

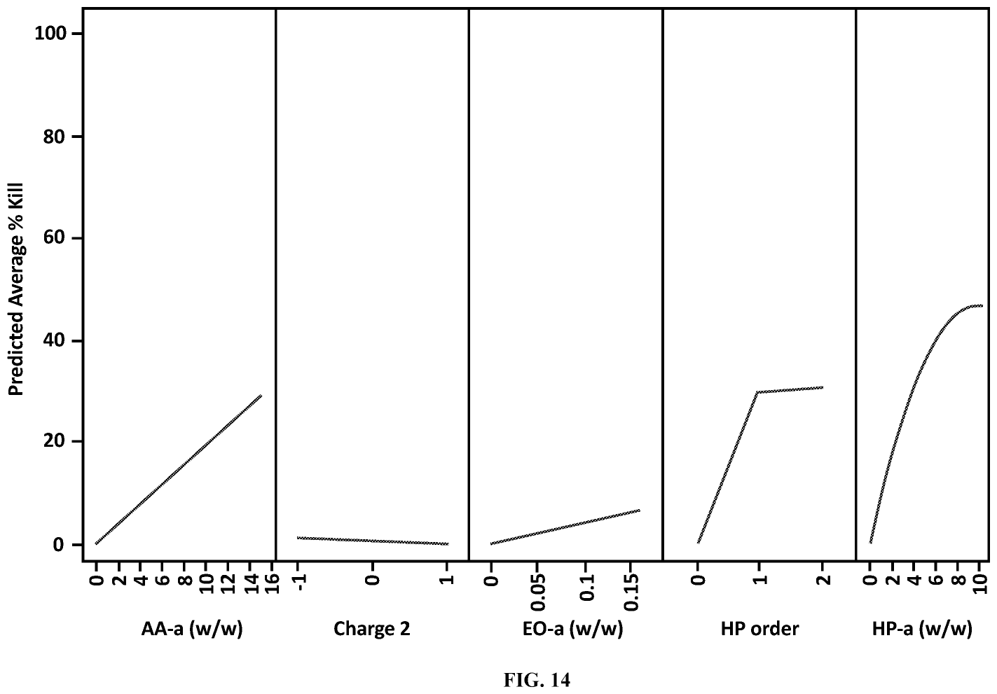

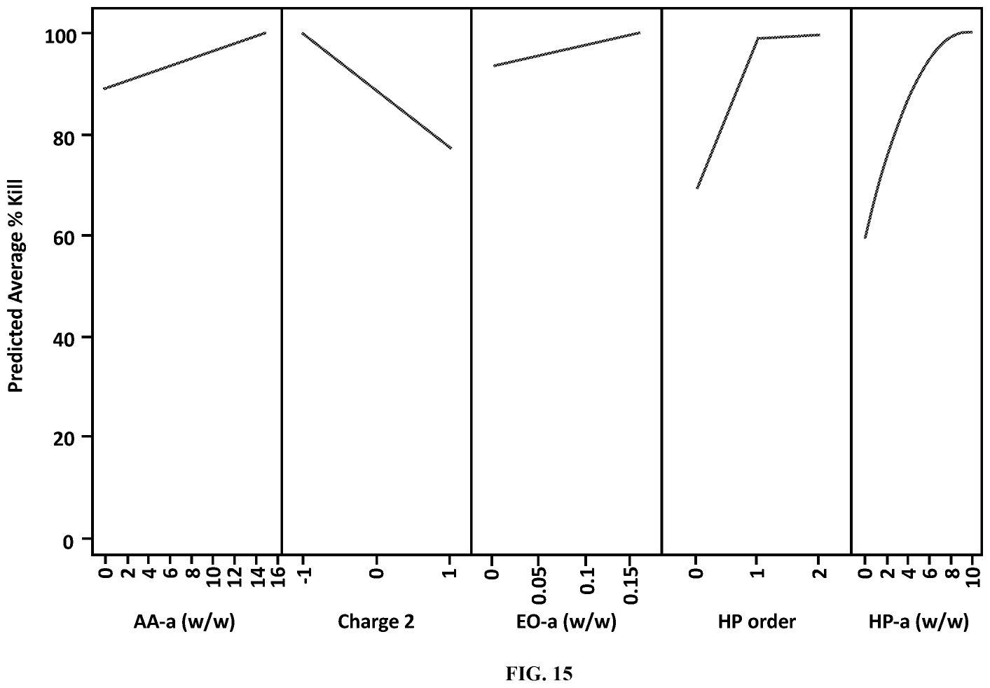

FIGS. 14 and 15 illustrate the effects on the percent kill of each of the components considered separately (FIG. 14) and when analyzed together (FIG. 15). In FIG. 14, when the actual concentrations of acetic acid (AA-a), cinnamon oil (EO-a), and hydrogen peroxide (HP-a) are all 0% by weight (w/w), the model predicts that the percent kill of the bacteria is 0. This result is equivalent to control reactions in which none of the reaction components are added. Although the plot for charge of the second aqueous composition (Charge 2) and order of addition (HP order) illustrate continuous lines, these plots are artifacts of the JMP program. For the charge of the second aqueous composition, a value of −1 indicates a negative charge, a value of 0 indicates a neutral charge, and a value of +1 indicates a positive charge. For the order of addition, an HP order value of 0 indicates that hydrogen peroxide is not present, an HP order value of 1 indicates that hydrogen peroxide was dispersed in the first aqueous composition, and an HP order value of 2 indicates that hydrogen peroxide was dispersed in the second aqueous composition. Not surprisingly, the addition of hydrogen peroxide has a more noticeable effect on the percent kill than does adding an equivalent amount of acetic acid. However, the effect of adding HP appears to level off at higher concentrations, whereas the correlation of adding more acetic acid appears to be linear. This phenomenon may indicate that acetic acid must be present at a concentration higher than that tested in these experiments in order to maximize the effect of hydrogen peroxide and cause the relationship between hydrogen peroxide concentration and percent kill to be more linear, if such a relationship exists. On the other hand, the leveling off at higher concentrations of hydrogen peroxide may indicate a quenching effect on the percent kill of the bacteria.

On the other hand, FIG. 15 illustrates the maximum effect that each reaction parameter has on the percent kill. In each case, where the plot for a particular reaction parameter reaches 100%, it indicates the optimum value for each variable, over all concentrations and reaction conditions tested. The value above each x-axis label indicates the optimum value for each variable. Interestingly, the optimum value for acetic acid and cinnamon oil concentrations sit at the maximum tested value (15% by weight of acetic acid, 0.16% by weight of cinnamon oil), indicating that higher concentrations of acetic acid and cinnamon oil can likely be used to have an even greater effect on killing bacteria. Surprisingly, while the plots of each of the variables generally have the same profile as in FIG. 14, the plot for the charge on the second aqueous composition illustrates a strong preference for being dispersed with a negative charge. This is true even though the percent kill is nearly identical whether the aqueous composition comprising hydrogen peroxide is dispersed first or second. Consequently, the abundance of electrons associated with dispersing the second aqueous composition with a negative charge appears to enhance the reactivity of the peracid as it is formed.

In a final set of experiments, given the statistically significant presence of cinnamon oil on the percent kill of bacteria, the concentration effects of cinnamon oil, as well as the effect of other natural biocides, was tested, using a similar procedure as above. The natural biocide was dispersed as part of the first aqueous composition along with acetic acid, and hydrogen peroxide was dispersed in the second aqueous composition. 16% by weight isopropyl alcohol (i-PrOH) was present in both aqueous compositions. Four different concentrations of cinnamon oil were tested: 0.065% by weight; 0.13% by weight; 0.20% by weight; and 0.26% by weight. Additionally, thyme oil (Thym), clove oil (Clov), and methylglyoxal (MGly) were also tested at 0.026% by weight in separate experiments. One experiment was conducted in which each of the four natural biocides were included in the first aqueous composition at a concentration of 0.065% by weight. Where present, hydrogen peroxide and acetic acid were typically added at 10% by weight, although in three of the experiments, they comprised only 5% by weight of their respective aqueous compositions. The reaction parameters and results are presented below in Table 7.

TABLE 7

| Exp. # | HP % (w/w) | AA % (w/w) | Cinn % (w/w) | Thym % (w/w) | Clov % (w/w) | Mgly % (w/w) | % Kill |
|---|---|---|---|---|---|---|---|
| 1 | 10 | 10 | 0 | 0 | 0 | 0 | 81.0 |
| 2 | 0 | 0 | 0.26 | 0 | 0 | 0 | 44.0 |
| 3 | 10 | 10 | 0.26 | 0 | 0 | 0 | 88.2 |
| 4 | 10 | 10 | 0 | 0.26 | 0 | 0 | 99.4 |
| 5 | 10 | 10 | 0 | 0 | 0.26 | 0 | 97.3 |
| 6 | 10 | 10 | 0 | 0 | 0 | 0.26 | 98.8 |
| 7 | 10 | 10 | 0.065 | 0.065 | 0.065 | 0.065 | 99.4 |
| 8 | 10 | 10 | 0.13 | 0 | 0 | 0 | 99.4 |
| 9 | 10 | 10 | 0.2 | 0 | 0 | 0 | 93.4 |
| 10 | 10 | 0 | 0 | 0 | 0 | 0 | 79.4 |
| 11 | 0 | 0 | 0.26 | 0 | 0 | 0 | 44.0 |
| 12 | 10 | 0 | 0.26 | 0 | 0 | 0 | 73.7 |
| 13 | 10 | 10 | 0.26 | 0 | 0 | 0 | 88.2 |
| 14 | 5 | 5 | 0.26 | 0 | 0 | 0 | 67.9 |
| 15 | 5 | 5 | 0 | 0 | 0 | 0 | 60.1 |
| 16 | 10 | 0 | 0.13 | 0 | 0 | 0 | 81.5 |
| 17 | 10 | 0 | 0.2 | 0 | 0 | 0 | 68.4 |
| 18 | 5 | 5 | 0.13 | 0 | 0 | 0 | 71.0 |

As illustrated in Table 7, reactions containing 10% by weight of hydrogen peroxide and acetic acid along with the highest concentrations of natural biocides had the strongest effect on the percent kill. Looking at Experiments 3 through 6, cinnamon oil was the weakest of the four natural biocides tested at 0.26% by weight, as thyme oil, clove oil, and methylglyoxal at the same concentration were all more effective than cinnamon oil. However, Experiment 8, in which cinnamon oil was present at only 0.13% by weight, was more effective than when cinnamon oil was included at 0.26% percent by weight, indicating a possible quenching issue at higher concentrations of cinnamon oil that are not exhibited by the other natural biocides. Nonetheless, the high effectiveness of compositions containing a natural biocide illustrates the effectiveness of including such compounds in at least one of the aqueous compositions according to methods of the present invention.

### Example 6: Effect of a Metal Halide on the Presence of Peracid on a Disinfected Surface

A study is conducted in accordance with embodiments of the present disclosure to determine the effect that a peracid scavenging composition comprising a metal halide compound has on the post-disinfection concentration of a peracid on a surface. The aqueous compositions of Example 2 are applied sequentially onto a surface using the same spraying protocol as used in Example 3. About one minute after the second aqueous composition is sprayed onto the surface and the peracid is formed in situ within the reaction layer, a peracid scavenging composition comprising 0.001 moles per liter is applied to the reaction layer using a hand sprayer, using the same hand spraying protocol as Example 3. It is expected that within 5 minutes, substantially all of the formed peracid will be removed from the surface.

I claim:

1. A method of disinfecting a surface in need of disinfecting within a volumetric space, comprising the steps of:
a) dispensing onto the surface a first aqueous composition comprising a first peracid reactant compound that is either a peroxide compound or an organic acid compound capable of reacting with a peroxide compound to form a peracid;
b) allowing a time sufficient for the first aqueous composition to distribute across the surface and coalesce into a first aqueous composition layer upon the surface;
c) dispensing onto the surface a second aqueous composition comprising a second peracid reactant compound that is the other of the first peracid reactant compound; and
d) allowing a second time sufficient for the second aqueous composition to combine with the coalesced first aqueous composition layer and to form a reaction layer upon the surface, thereby forming a peracid in situ within the reaction layer and disinfecting the surface.

2. The method of claim 1, wherein the volumetric space is accessible to at least one of humans and animals.

3. The method of either claim 1 or claim 2, wherein substantially all of the first aqueous composition is retained on the surface upon dispensing the second aqueous composition onto the surface.

4. The method of any of claims 1-3, wherein the first aqueous composition and the second aqueous composition are each dispensed as a liquid stream onto the surface.

5. The method of claim 4, wherein the method further comprises the step of providing a mechanical coarse spray device, wherein the first aqueous composition and the second aqueous composition are each dispensed as a liquid stream onto the surface using the mechanical coarse spray device; preferably wherein the liquid stream is dispensed in the form of a mist, a shower, or a jet.

6. The method of any of claims 1-5, wherein the time sufficient for the first aqueous composition to distribute across the surface is the time sufficient to fully immerse the surface with the first aqueous composition.

7. The method of any of claims 1-6, wherein the second time sufficient for the second aqueous composition to distribute across the surface is the time sufficient to fully immerse the surface with the second aqueous composition.

8. The method of any of claims 1-7, wherein the first aqueous composition and the second aqueous composition are substantially free of surfactants, polymers, chelators, and metal colloids or nanoparticles.

9. The method of any of claims 1-8, wherein a stoichiometric amount of the dispersed peroxide compound is equal to or greater than a stoichiometric amount of the dispersed organic acid compound.

10. The method of any of claims 1-9, wherein the pH of the aqueous composition comprising the organic acid compound is less than or equal to about 7.

11. The method of any of claims 1-10, wherein:
a) the first peracid reactant compound is a peroxide compound, preferably hydrogen peroxide, and
b) the second peracid reactant compound is an organic acid compound; preferably an organic carboxylic acid selected from the group consisting of formic acid, acetic acid, citric acid, succinic acid, oxalic acid, propanoic acid, lactic acid, butanoic acid, pentanoic acid, octanoic acid, and a mixture thereof; and more preferably acetic acid.

12. The method of any of claims 1-11, wherein the first aqueous composition comprises at least about 2% by weight, and up to about 15% by weight, hydrogen peroxide.

13. The method of any of claims 1-12, wherein the second aqueous composition comprises at least about 1% by weight, and up to about 10% by weight, acetic acid.

14. The method of any of claims 1-13, wherein at least one of the first aqueous composition and the second aqueous composition further comprises an alcohol, preferably at least about 1% by weight, and up to about 30% by weight, alcohol.

15. The method of claim 14, wherein the alcohol comprises a lower-chain alcohol selected from the group consisting of ethanol, isopropanol, t-butanol, and mixtures thereof, preferably isopropanol.

16. The method of any of claims 1-15, wherein at least one of the first aqueous composition or the second aqueous composition comprises about 0.001% to about 1% by weight of a natural biocide selected from the group consisting of manuka honey and the essential oils of oregano, thyme, lemongrass, lemons, oranges, anise, cloves, aniseed, cinnamon, geraniums, roses, mint, peppermint, lavender, citronella, eucalyptus, sandalwood, cedar, rosmarin, pine, vervain fleagrass, and ratanhiae, and combinations thereof.

17. The method of any of claims 1-15, wherein at least one of the first aqueous composition or the second aqueous composition comprises about 0.001% to about 1% by weight of a natural biocidal compound selected from the group consisting of methylglyoxal, carvacrol, eugenol, linalool, thymol, p-cymene, myrcene, borneol, camphor, caryophillin, cinnamaldehyde, geraniol, nerol, citronellol, and menthol, and combinations thereof.

18. The method of any of claims 1-17, wherein the method further includes the step of illuminating at least one of the first aqueous composition, the second aqueous composition, and the reaction layer with a wavelength consisting essentially of ultraviolet light.

19. The method of any of claims 1-18, wherein the surface in need of disinfecting is selected from the group consisting of: plastics, metals, Linoleum; tiles, vinyl, stone, wood, concrete, wallboards, plaster, pulp and fiber-based materials, glass, heating, ventilation, and air conditioning (HVAC) systems, plumbing, vinyl, and a combination thereof.

20. A method of disinfecting a surface in need of disinfecting within a volumetric space, comprising the steps of:
a) dispersing into the volumetric space a multiplicity of microdroplets of a first aqueous composition comprising a first peracid reactant compound that is either a peroxide compound or an organic acid compound capable of reacting with a peroxide compound to form a peracid;
b) allowing a time sufficient for the multiplicity of microdroplets of the first aqueous composition to distribute throughout the volumetric space and to deposit and coalesce into a first aqueous composition layer upon the surface;
c) dispersing into the volumetric space a multiplicity of microdroplets of a second aqueous composition comprising a second peracid reactant compound that is the other of the first peracid reactant compound; and
d) allowing a second time sufficient for the multiplicity of microdroplets of the second aqueous composition to deposit onto the coalesced first aqueous composition layer to form a reaction layer upon the surface, thereby forming a peracid in situ within the reaction layer and disinfecting the surface;
wherein the method further includes the steps of dispersing into the volumetric space one or more supplemental aqueous compositions and allowing a time sufficient for each dispersed supplemental aqueous composition to distribute throughout the volumetric space and to deposit onto the surface.

21. The method of claim 20, wherein a supplemental aqueous composition is dispersed into the volumetric space at a time selected from the group consisting of: prior to dispersing the first aqueous composition into the volumetric space; after the first aqueous composition layer is formed upon the surface and prior to dispersing the second aqueous composition into the volumetric space; after the peracid has been formed in situ within the reaction layer on the surface; and a combination thereof.

22. The method of claim 21, wherein each supplemental aqueous composition is selected from the group consisting of a peracid scavenging composition, a pesticide composition, and an environmental conditioning composition.

23. The method of claim 22, wherein a peracid scavenging composition comprising a metal halide compound is dispersed after the peracid has been formed in situ within the reaction layer on the surface, wherein the metal halide compound comprises iodide or chloride, preferably a metal halide compound selected from the group consisting of potassium iodide, potassium chloride, and sodium chloride, and more preferably potassium iodide.

24. The method of claim 23, wherein the peracid scavenging composition comprises at least about 0.0001 moles per liter, and up to about 1 mole per liter, potassium iodide.

25. The method of claim 23, wherein a stoichiometric amount of the metal halide compound is dispersed that is equal to or greater than a stoichiometric amount of the peracid formed in situ within the reaction layer, thereby scavenging substantially all of the formed peracid from the surface.

26. The method of claim 22, wherein the pesticide composition comprises at least one of a fungicide, a rodenticide, a herbicide, a larvicide, an insecticide, and a combination thereof, and preferably an insecticide configured to kill bed bugs or termites.

27. The method of claim 26, wherein the pesticide composition is dispersed into the volumetric space prior to dispersing the first aqueous composition into the volumetric space.

28. The method of claim 26, wherein the pesticide composition is dispersed into the volumetric space after the peracid has been formed in situ within the reaction layer on the surface.

29. The method of claim 22, wherein the environmental conditioning composition consists essentially of water.

30. The method of claim 29, wherein the environmental conditioning composition is dispersed into the volumetric space prior to dispersing the first aqueous composition into the volumetric space, and the time sufficient for the environmental conditioning composition to distribute throughout the volumetric space is the time sufficient to cause the volumetric space to have a relative humidity of at least about 50 percent, and up to about 99 percent.

31. The method of claim 29, wherein the environmental conditioning composition is dispersed into the volumetric space after the first aqueous composition layer is formed upon the surface and prior to dispersing the second aqueous composition into the volumetric space.

32. The method of claim 29, wherein the environmental conditioning composition is dispersed into the volumetric space after the peracid has been formed in situ within the reaction layer on the surface.

33. The method of claim 22, wherein the environmental conditioning composition further consists essentially of a fragrant compound, and the environmental conditioning composition is dispersed into the volumetric space after the peracid has been formed in situ within the reaction layer on the surface.

34. The method of claim 33, wherein the fragrant compound is selected from the group consisting of methylglyoxal, carvacrol, eugenol, linalool, thymol, p-cymene, myrcene, borneol, camphor, caryophillin, cinnamaldehyde, geraniol, nerol, citronellol, and menthol, including combinations thereof.

35. The method of any of claims 20-34, wherein one or more of the supplemental aqueous compositions are dispersed into the volumetric space as a multiplicity of microdroplets.

36. The method of claim 35, wherein the multiplicity of microdroplets of the supplemental aqueous composition is electrostatically charged.

37. The method of claim 36, wherein the electrostatically-charged microdroplets of the supplemental aqueous composition are negatively charged.

38. The method of claim 35, wherein the multiplicity of microdroplets of at least one of the first aqueous composition, second aqueous composition, or the one or more supplemental aqueous compositions is formed by first heating the aqueous composition to produce a vapor and allowing a time sufficient for the vapor to distribute throughout the volumetric space and to cool and condense into microdroplets.

39. The method of any of claims 20-38, wherein the first aqueous composition and the second aqueous composition are substantially free of surfactants, polymers, chelators, and metal colloids or nanoparticles.

40. The method of any of claims 20-39, wherein a stoichiometric amount of the dispersed peroxide compound is equal to or greater than a stoichiometric amount of the dispersed organic acid compound.

41. The method of any of claims 20-40, wherein the pH of the aqueous composition comprising the organic acid compound is less than or equal to about 7.

42. The method of any of claims 20-41, wherein:
a) the first peracid reactant compound is a peroxide compound, preferably hydrogen peroxide, and
b) the second peracid reactant compound is an organic acid compound; preferably an organic carboxylic acid selected from the group consisting of: formic acid, acetic acid, citric acid, succinic acid, oxalic acid, propanoic acid, lactic acid, butanoic acid, pentanoic acid, and octanoic acid; and more preferably acetic acid.

43. The method of any of claims 20-42, wherein the first aqueous composition comprises at least about 1% by weight, and up to about 25% by weight, hydrogen peroxide.

44. The method of any of claims 20-43, wherein the second aqueous composition comprises at least about 1% by weight acetic acid, and up to about 25% by weight, acetic acid.

45. The method of any of claims 20-44, wherein at least one of the first aqueous composition and the second aqueous composition further comprises an alcohol, preferably at least about 1% by weight, and up to about 30% by weight, alcohol.

46. The method of claim 45, wherein the alcohol comprises a lower-chain alcohol selected from the group consisting of ethanol, isopropanol, t-butanol, and mixtures thereof, preferably isopropanol.

47. The method of any of claims 20-46, wherein at least one of the first aqueous composition or the second aqueous composition comprises about 0.001% to about 1% by weight of a natural biocide selected from the group consisting of manuka honey and the essential oils of oregano, thyme, lemongrass, lemons, oranges, anise, cloves, aniseed, cinnamon, geraniums, roses, mint, peppermint, lavender, citronella, eucalyptus, sandalwood, cedar, rosmarin, pine, vervain fleagrass, and ratanhiae, and combinations thereof.

48. The method of any of claims 20-46, wherein at least one of the first aqueous composition or the second aqueous composition comprises about 0.001% to about 1% by weight of a natural biocidal compound selected from the group consisting of methylglyoxal, carvacrol, eugenol, linalool, thymol, p-cymene, myrcene, borneol, camphor, caryophillin, cinnamaldehyde, geraniol, nerol, citronellol, and menthol, and combinations thereof.

49. The method of any of claims 20-48, wherein the method further includes the step of illuminating at least one of the first aqueous composition, the second aqueous composition, and the reaction layer with a wavelength consisting essentially of ultraviolet light.

50. A method of disinfecting a surface in need of disinfecting within a volumetric space, comprising the steps of:
a) dispersing into the volumetric space a multiplicity of microdroplets of a first aqueous composition comprising a peracid, and
b) allowing a time sufficient for the first aqueous composition to distribute throughout the volumetric space and to deposit onto the surface, thereby disinfecting the surface;
wherein the method further includes the step of dispersing into the volumetric space a multiplicity of microdroplets of one or more supplemental aqueous compositions selected from the group consisting of a peracid scavenging composition, a pesticide composition, and an environmental conditioning composition, and allowing a time sufficient for each dispersed supplemental aqueous composition to distribute throughout the volumetric space and to deposit onto the surface.

51. The method of claim 50, wherein the peracid is peroxyacetic acid.

52. The method of either claim 50 or claim 51, wherein a peracid scavenging composition comprising a metal halide compound is dispersed after the first aqueous composition has deposited onto the surface, wherein the metal halide compound comprises iodide or chloride, preferably a metal halide compound selected from the group consisting of potassium iodide, potassium chloride, and sodium chloride, and more preferably potassium iodide.

53. The method of claim 52, wherein the peracid scavenging composition comprises less than about 6 moles per liter of potassium iodide, including at least about 0.0001 moles per liter, and up to about 1 mole per liter, potassium iodide.

54. The method of claim 52, wherein a stoichiometric amount of the metal halide compound is dispersed into the volumetric space that is equal to or greater than a stoichiometric amount of the peracid dispersed into the volumetric space, thereby scavenging substantially all of the peracid from the volumetric space.

55. The method of either claim 50 or 51, wherein the pesticide composition comprises at least one fungicide, rodenticide, herbicide, larvicide, or insecticide, including combinations thereof, preferably an insecticide configured to kill bed bugs or termites.

56. The method of claim 55, wherein the pesticide composition is dispersed into the volumetric space prior to dispersing the first aqueous composition into the volumetric space.

57. The method of claim 55, wherein the pesticide composition is dispersed into the volumetric space after the first aqueous composition has deposited onto the surface.

58. The method of either claim 50 or 51, wherein the environmental conditioning composition consists essentially of water.

59. The method of claim 58, wherein the environmental conditioning composition is dispersed into the volumetric space prior to dispersing the first aqueous composition into the volumetric space, and the method further includes the step of allowing a time sufficient for the environmental conditioning composition to distribute throughout the volumetric space and cause the volumetric space to have a relative humidity of at least about 50 percent, and up to about 95 percent.

60. The method of claim 58, wherein the environmental conditioning composition is dispersed into the volumetric space after the first aqueous composition has deposited onto the surface.

61. The method of either claim 50 or 51, wherein the environmental conditioning composition further consists essentially of a fragrant compound, and the environmental conditioning composition is dispersed into the volumetric space after the first aqueous composition has deposited onto the surface.

62. The method of claim 61, wherein the fragrant compound is selected from the group consisting of methylglyoxal, carvacrol, eugenol, linalool, thymol, p-cymene, myrcene, borneol, camphor, caryophillin, cinnamaldehyde, geraniol, nerol, citronellol, and menthol, including combinations thereof.

63. The method of any of claims 50-62, wherein the multiplicity of microdroplets of the first aqueous composition is electrostatically charged.

64. The method of claim 63, wherein the electrostatically-charged microdroplets of the first aqueous composition are negatively charged.

65. The method of any of claims 50-62, wherein the multiplicity of microdroplets of at least one of the first aqueous composition or the one or more supplemental aqueous compositions is formed by first heating the aqueous composition to produce a vapor and allowing a time sufficient for the vapor to distribute throughout the volumetric space and to cool and condense into microdroplets.

66. The method of any of claims 50-65, wherein the method further includes the step of illuminating at least one of the first aqueous composition and the surface with a wavelength consisting essentially of ultraviolet light.

67. A method of disinfecting a surface in need of disinfecting within a volumetric space, comprising the steps of:
a) dispensing onto the surface a quantity of a first aqueous composition comprising a first peracid reactant compound that is either a peroxide compound or an organic acid compound capable of reacting with a peroxide compound to form a peracid;
b) allowing a time sufficient for the first aqueous composition to deposit onto the surface and coalesce into a first aqueous composition layer upon the surface, wherein the time sufficient is at least about 30 seconds, and up to at least about 15 minutes;
c) dispensing onto the surface a quantity of a second aqueous composition comprising a second peracid reactant compound that is the other of the first peracid reactant compound; and
d) allowing a second time sufficient for the second aqueous composition to deposit onto the surface and combine with the coalesced first aqueous composition layer to form a reaction layer upon the surface, wherein the second time sufficient is at least about 30 seconds, and up to at least about 15 minutes, thereby forming a peracid in situ within the reaction layer and disinfecting the surface.

68. The method of claim 67, wherein the volumetric space is enterable by at least one of humans and animals.

69. The method of either claim 67 or claim 68, wherein substantially all of the first aqueous composition is retained on the surface upon dispensing the second aqueous composition onto the surface.

70. The method of any of claims 67-69, wherein the first aqueous composition and the second aqueous composition are each dispensed as a liquid stream onto the surface.

71. The method of any of claims 67-69, wherein the first aqueous composition and the second aqueous composition are each dispensed as a multiplicity of microdroplets onto a surface, wherein a preponderance of the multiplicity of microdroplets of the first aqueous composition dispersed into the volumetric space has an effective diameter of at least about 5 microns, and up to about 100 microns, preferably an effective diameter of about 10 microns to about 25 microns, and more preferably an effective diameter of about 15 microns.

72. The method of claim 71, wherein the quantity of the dispersed first aqueous composition is sufficient to provide the coalesced layer of the first aqueous composition with an effective uniform thickness of at least about 1 micron and up to about 20 microns, and preferably an effective uniform thickness of about 3 microns to about 8 microns.

73. The method of either claim 71 or claim 72, wherein the quantity of the dispersed second aqueous composition is sufficient to provide the reaction layer with an effective uniform thickness of at least about 1 micron and up to about 20 microns, and preferably an effective uniform thickness of about 3 microns to about 8 microns.

74. The method of any of claims 67-73, wherein the first aqueous composition and the second aqueous composition are substantially free of surfactants, polymers, chelators, and metal colloids or nanoparticles.

75. The method of any of claims 67-74, wherein a stoichiometric amount of the dispersed peroxide compound is equal to or greater than a stoichiometric amount of the dispersed organic acid compound.

76. The method of any of claims 67-75, wherein the pH of the aqueous composition comprising the organic acid compound is less than or equal to about 7.

77. The method of any of claims 67-76, wherein:
a) the first peracid reactant compound is a peroxide compound, preferably hydrogen peroxide, and
b) the second peracid reactant compound is an organic acid compound; preferably an organic carboxylic acid selected from the group consisting of: formic acid, acetic acid, citric acid, succinic acid, oxalic acid, propanoic acid, lactic acid, butanoic acid, pentanoic acid, and octanoic acid; and more preferably acetic acid.

78. The method of any of claims 67-77, wherein the first aqueous composition comprises at least about 1% by weight, and up to about 20% by weight, hydrogen peroxide.

79. The method of any of claims 67-78, wherein the second aqueous composition comprises at least about 2% by weight, and up to about 25% by weight, acetic acid.

80. The method of any of claims 67-79, wherein at least one of the first aqueous composition and the second aqueous composition further comprises an alcohol, preferably at least about 1% by weight, and up to about 40% by weight, alcohol.

81. The method of claim 80, wherein the alcohol comprises a lower-chain alcohol selected from the group consisting of ethanol, isopropanol, t-butanol, and mixtures thereof, preferably isopropanol.

82. The method of any of claims 67-81, wherein at least one of the first aqueous composition or the second aqueous composition comprises about 0.001% to about 1% by weight of a natural biocide selected from the group consisting of manuka honey and the essential oils of oregano, thyme, lemongrass, lemons, oranges, anise, cloves, aniseed, cinnamon, geraniums, roses, mint, peppermint, lavender, citronella, eucalyptus, sandalwood, cedar, rosmarin, pine, vervain fleagrass, and ratanhiae, and combinations thereof.

83. The method of any of claims 67-81, wherein at least one of the first aqueous composition or the second aqueous composition comprises about 0.001% to about 1% by weight of a natural biocidal compound selected from the group consisting of methylglyoxal, carvacrol, eugenol, linalool, thymol, p-cymene, myrcene, borneol, camphor, caryophillin, cinnamaldehyde, geraniol, nerol, citronellol, and menthol, and combinations thereof.

84. The method of any of claims 67-83, wherein the method further includes the step of illuminating at least one of the first aqueous composition, the second aqueous composition, and the reaction layer with a wavelength consisting essentially of ultraviolet light.

85. A sequential application and delivery system for sequentially dispensing a first aqueous composition and a second aqueous composition, comprising:
a) a plurality of aqueous composition containers, each configured for housing or containing an aqueous composition;
b) a plurality of pumps, each pump in fluid communication respectively with one of the aqueous composition containers therewith; and,
c) one or more aqueous composition delivery nozzles, each aqueous composition delivery nozzle in fluid communication with at least one pump and configured to sequentially dispense one or more aqueous compositions into a volumetric space.

86. The sequential application and delivery system of claim 85, further comprising a data acquisition and control system including:
a) a means for detecting the volume of the aqueous composition within each of the aqueous composition containers;
b) a data acquisition bus;
c) a control bus; and
d) a controller electrically coupled to the aqueous composition containers and configured to read the means for detecting the volume of the aqueous composition within each of the aqueous composition containers.

87. The sequential application and delivery system of claim 86, wherein such means for detecting the volume of the aqueous composition include float, capacitance, conductivity, ultrasonic, radar level, and optical sensors.

88. The sequential application and delivery system of either claim 86 or 87, wherein each pump includes a drive electrically coupled to the controller through the control bus, wherein the drive is configured to engage the pumps to dispense aqueous compositions from the aqueous composition containers to and through the aqueous composition delivery nozzles into the volumetric space.

89. The sequential application and delivery system of any of claims 86-88, further comprising one or more sensors proximate or adjacent to the volumetric space and in data communication with the data acquisition bus, wherein the at least one sensor comprises a means for detecting at least one environmental condition within the volumetric space, selected from the group consisting of motion detectors, global positioning system (GPS) detectors, infrared sensors, audio sensors, thermal sensors, accelerometers, cameras, or light sensors, preferably laser light sensors, including combinations thereof.

90. The sequential application and delivery system of claim 89, wherein the controller is programmed to cease dispensing an aqueous composition upon a sensor detecting the presence of an animal or human within the volumetric space.

91. The sequential application and delivery system of claim 89, wherein the sensor is configured to detect the Cartesian dimensions of the volumetric space and communicate the detected dimensions to the controller through the data acquisition bus.

92. The sequential application and delivery system of any of claims 86-91, wherein the controller is programmed to delay for a defined time after dispensing the first aqueous composition into the volumetric space before dispensing the second aqueous composition into the volumetric space.

93. The sequential application and delivery system of any of claims 85-92, wherein a portion of the sequential application and delivery system is coupled to a mobilized conveyance selected from the group consisting of a hand-carried dispensing unit, backpack, cart, trolley, preferably an optically-controlled or directed trolley, robot, or drone.

94. The sequential application and delivery system of any of claims 85-93, further comprising an ionizing device proximate or adjacent to one or more nozzles, the ionizing device configured to electrostatically charge a quantity of the aqueous composition dispensed by the one or more nozzles.

95. The sequential application and delivery system of any of claims 85-93, further comprising a vaporizer that is located proximate or adjacent to one or more nozzles and is electrically coupled and responsive to the controller, wherein the controller is programmed to energize the vaporizer and cause the vaporizer to emit a hot gaseous stream at the aqueous composition after being dispensed from the nozzle.

96. A sequential application and delivery system for sequentially dispensing a plurality of aqueous compositions, including a first aqueous composition and a second aqueous composition, wherein the first aqueous composition comprises a peracid reactant compound selected from the group consisting of a peroxide compound and an organic acid compound that is capable of reacting with the peroxide compound to form a peracid, and the second aqueous composition comprises the peracid reactant compound that is the other of the first peracid reactant compound, the sequential application and delivery system comprising:
a) a plurality of aqueous composition containers, each configured for housing or containing an aqueous composition;
b) a plurality of pumps, each pump in fluid communication respectively with one of the aqueous composition containers therewith;
c) one or more aqueous composition delivery nozzles, each aqueous composition delivery nozzle in fluid communication with at least one pump and configured to sequentially dispense one or more aqueous compositions into a volumetric space; and
wherein the sequential application and delivery system is configured to prevent the first aqueous composition and the second aqueous composition from contacting each other until after each aqueous composition is dispensed into the volumetric space.

97. The sequential application and delivery system of claim 96, wherein the peroxide compound is hydrogen peroxide.

98. The sequential application and delivery system of claim 96 or 97, wherein the organic acid compound is acetic acid.

99. The sequential application and delivery system of any of claims 96-98, wherein the sequential application and delivery system is configured to dispense the first aqueous composition and the second aqueous composition onto one or more surfaces within the volumetric space, thereby forming a peracid in situ on the surfaces.

100. The sequential application and delivery system of any of claims 96-99, further comprising a data acquisition and control system including:
a) a means for detecting the volume of the aqueous composition within each of the aqueous composition containers;
b) a data acquisition bus;
c) a control bus; and
d) a controller electrically coupled to the aqueous composition containers and configured to read the means for detecting the volume of the aqueous composition within each of the aqueous composition containers.

101. The sequential application and delivery system of claim 100, wherein such means for detecting the volume of the aqueous composition include float, capacitance, conductivity, ultrasonic, radar level, and optical sensors.

102. The sequential application and delivery system of claim 100 or 101, wherein each pump includes a drive electrically coupled to the controller through the control bus, wherein the drive is configured to engage the pumps to dispense aqueous compositions from the aqueous composition containers to and through the aqueous composition delivery nozzles into the volumetric space.

103. The sequential application and delivery system of any of claims 100-102, further comprising one or more sensors proximate or adjacent to the volumetric space and in data communication with the data acquisition bus, wherein the at least one sensor comprises a means for detecting at least one environmental condition within the volumetric space, selected from the group consisting of motion detectors, global positioning system (GPS) detectors, infrared sensors, audio sensors, thermal sensors, accelerometers, cameras, or light sensors, preferably laser light sensors, including combinations thereof.

104. The sequential application and delivery system of claim 103, wherein the controller is programmed to cease dispensing an aqueous composition upon a sensor detecting the presence of an animal or human within the volumetric space.

105. The sequential application and delivery system of claim 103, wherein the sensor is configured to detect the Cartesian dimensions of the volumetric space and communicate the detected dimensions to the controller through the data acquisition bus.

106. The sequential application and delivery system of any of claims 100-105, wherein the controller is programmed to delay for a time sufficient for the first aqueous composition to distribute throughout the volumetric space and to deposit and coalesce into a layer onto one or more surfaces within the volumetric space before dispensing the second aqueous composition into the volumetric space.

107. The sequential application and delivery system of any of claims 96-106, wherein a portion of the sequential application and delivery system is coupled to a mobilized conveyance selected from the group consisting of a hand-carried dispensing unit, backpack, cart, trolley, preferably an optically-controlled or directed trolley, robot, or drone.

108. The sequential application and delivery system of any of claims 96-107, further comprising an ionizing device proximate or adjacent to one or more nozzles, the ionizing device configured to electrostatically charge a quantity of the first aqueous composition and/or the second aqueous composition dispensed by the sequential application and delivery system.

109. The sequential application and delivery system of claim 108, wherein the controller is programmed to dispense the first aqueous composition as negatively-charged droplets.

110. The sequential application and delivery system of claim 108, wherein the controller is programmed to dispense the first aqueous composition as positively-charged droplets.

111. The sequential application and delivery system of claim 109 or 110, wherein the controller is programmed to dispense the second aqueous composition as electrostatically-charged droplets having the opposite polarity as the first aqueous composition.

112. The sequential application and delivery system of any of claims 96-107, further comprising a vaporizer that is located proximate or adjacent to one or more nozzles and is electrically coupled and responsive to the controller, wherein the controller is programmed to energize the vaporizer and cause the vaporizer to emit a hot gaseous stream at the aqueous composition after being dispensed from the nozzle.

113. The sequential application and delivery system of any of claims 85-112, further comprising an Internet of Things (IoT) configured to engage one or more of the plurality of pumps in a sequential, timed manner.

114. The sequential application and delivery system of claim 113, wherein the IoT comprises one or more remotely-controlled outlets in direct wireless electronic communication with the Internet and configured for sequentially energizing the one or more of the plurality of pumps.

115. The sequential application and delivery system of claim 114, wherein the IoT further comprises:
a) at least one of a mobile device and a computer in electronic communication with the Internet, each including:
i) an operating system;
ii) a home automation application configured to run on the operating system; and,
iii) a routine created within the home automation application and configured to actuate the one or more remotely controlled outlets to engage the one or more of the plurality of pumps in a sequential timed manner.

116. The sequential application and delivery system of claim 115, wherein the IoT further comprises one or more sensors in direct wireless electronic communication with the Internet and configured to sense environmental conditions within the volumetric space, selected from the group consisting of: motion detectors; global positioning system detectors; infrared sensors; audio sensors; thermal sensors; accelerometers; light sensors, preferably laser light sensors; and cameras;
including combinations thereof.

117. The sequential application and delivery system of any of claims 113-116, wherein the IoT further comprises at least two remotely-controlled outlets in direct wireless electronic communication with the Internet, each remotely-controlled outlet configured for sequentially energizing at least one of the plurality of pumps.

118. The sequential application and delivery system of any of claims 113-116, wherein the sequential application and delivery system comprises a single aqueous composition delivery nozzle.

119. The sequential application and delivery system of claim 113, wherein the IoT comprises one or more remotely controlled outlets in wireless electronic communication with an intranet and configured for sequentially energizing one or more of the plurality of pumps.

120. The sequential application and delivery system of claim 119, wherein the IoT further comprises:
a) a hub in electronic communication with the intranet, including:
i) an operating system;
ii) a home automation application configured to run on the operating system; and,
iii) a routine created within the home automation application and configured to actuate the one or more remotely controlled outlets to engage the one or more of the plurality of pumps in a sequential timed manner.

121. The sequential application and delivery system of either claim 119 or 120, wherein the IoT further comprises:
a) a mobile device in electronic communication with the intranet, including:
i) an operating system;
ii) a home automation application configured to run on the operating system; and,
iii) a routine created within the home automation application and configured to actuate the one or more remotely controlled outlets to engage the one or more of the plurality of pumps in a sequential timed manner.

122. The sequential application and delivery system of any of claims 119-121, wherein the IoT further comprises one or more sensors in direct wireless electronic communication with the intranet and configured to sense environmental conditions within the volumetric space, selected from the group consisting of: motion detectors; global positioning system detectors; infrared sensors; audio sensors; thermal sensors; accelerometers; light sensors, preferably laser light sensors; and cameras; including combinations thereof.

123. The sequential application and delivery system of any of claims 119-122, wherein the IoT further comprises at least two remotely-controlled outlets in direct wireless electronic communication with the intranet, each remotely-controlled outlet configured for sequentially energizing at least one of the plurality of pumps.

124. The sequential application and delivery system of any of claims 119-122, wherein the sequential application and delivery system comprises a single aqueous composition delivery nozzle.

125. The sequential application and delivery system of any of claims 85-112, further comprising a single board computer assembly (SBC) configured to engage one or more of the plurality of pumps in a sequential timed manner.

126. The sequential application and delivery system of claim 125, the SBC comprising a hardware attached on top (HAT) circuit board having one or more relays, each relay respectively associated with one or more of the plurality of pumps and configured to pass electric power to the respective one or more of the plurality of pumps in a sequential timed manner.

127. The sequential application and delivery system of claim 126, the SBC further comprising a display, the display having a user interface for energizing one or more of the plurality of pumps in a sequential timed manner.

128. The sequential application and delivery system of any of claims 125-127, further comprising a mobile device configured for energizing one or more of the plurality of pumps in a sequential timed manner.

129. The sequential application and delivery system of any of claims 125-128, wherein the SBC comprises a HAT circuit board having at least two relays, each relay respectively associated with one or more of the plurality of pumps and configured to pass electric power to one or more of the plurality of pumps in a sequential timed manner.

130. A kit for use in disinfecting a surface in need of disinfecting within a volumetric space, comprising:
a) a first aqueous composition comprising a first peracid reactant compound that is either a peroxide compound or an organic acid compound capable of reacting with a peroxide compound to form a peracid;
b) a second aqueous composition comprising a second peracid reactant compound that is the other of the first peracid reactant compound; and
c) instructions comprising the method of any of claims 1-84,
wherein the kit is arranged such that the first aqueous composition and the second aqueous composition are packaged separately and are not combined until the first aqueous composition and the second aqueous composition are applied sequentially onto the surface to form a reaction layer comprising the first aqueous composition and the second aqueous composition, thereby forming a peracid in situ within the reaction layer and disinfecting the surface.

131. The kit of claim 130, wherein the kit further comprises any of the sequential application and delivery systems of claims 85-129.

132. The kit of either claim 130 or claim 131, wherein the first aqueous composition and the second aqueous composition are substantially free of surfactants, polymers, chelators, and metal colloids or nanoparticles.

133. The kit of any of claims 130-132, wherein the pH of the aqueous composition comprising the organic acid compound is less than or equal to about 7.

134. The kit of any of claims 130-133, wherein:
a) the first peracid reactant compound is a peroxide compound, preferably hydrogen peroxide, and
b) the second peracid reactant compound is an organic acid compound; preferably an organic carboxylic acid selected from the group consisting of: formic acid, acetic acid, citric acid, succinic acid, oxalic acid, propanoic acid, lactic acid, butanoic acid, pentanoic acid, and octanoic acid; and more preferably acetic acid.

135. The kit of any of claims 130-134, wherein the first aqueous composition comprises at least about 1% by weight, and up to about 15% by weight, hydrogen peroxide.

136. The kit of any of claims 130-135, wherein the second aqueous composition comprises at least about 1% by weight, and up to about 15% by weight, acetic acid.

137. The kit of any of claims 130-136, wherein at least one of the first aqueous composition and the second aqueous composition further comprises an alcohol, preferably at least about 1% by weight, and up to about 40% by weight alcohol.

138. The kit of claim 137, wherein the alcohol comprises a lower-chain alcohol selected from the group consisting of ethanol, isopropanol, t-butanol, and mixtures thereof, preferably isopropanol.

139. The kit of any of claims 130-138, wherein at least one of the first aqueous composition or the second aqueous composition comprises about 0.001% to about 1% by weight of a natural biocide selected from the group consisting of manuka honey and the essential oils of oregano, thyme, lemongrass, lemons, oranges, anise, cloves, aniseed, cinnamon, geraniums, roses, mint, peppermint, lavender, citronella, eucalyptus, sandalwood, cedar, rosmarin, pine, vervain fleagrass, and ratanhiae, and combinations thereof.

140. The kit of any of claims 130-138, wherein at least one of the first aqueous composition or the second aqueous composition comprises about 0.001% to about 1% by weight of a natural biocidal compound selected from the group consisting of methylglyoxal, carvacrol, eugenol, linalool, thymol, p-cymene, myrcene, borneol, camphor, caryophillin, cinnamaldehyde, geraniol, nerol, citronellol, and menthol, and combinations thereof.
